(19) United States
(12) Patent Application Publication
(10) Pub. No.: US 2007/0016453 A1
(43) Pub. Date: Jan. 18, 2007

(54) Medical information management system and medical information management method

(75) Inventors: Keiichi Hiyama, Tokyo (JP); Shinichi Omori, Tokyo (JP); Ryoichi Hosoya, Chiba (JP); Kazuhiro Akiyama, Tokyo (JP); Hidenobu Kubo, Tokyo (JP); Naoki Hosaka, Tokyo (JP); Tatsuya Shiobara, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: Olympus Medical Systems Corporation, Tokyo (JP)

(21) Appl. No.: 11/485,571

(22) Filed: Jul. 12, 2006

(30) Foreign Application Priority Data
Jul. 12, 2005 (JP) 2005-202717
Aug. 10, 2005 (JP) 2005-232343

(51) Int. Cl. G06F 19/00; A61B 5/00

(52) U.S. Cl. 705/3; 600/300

(57) ABSTRACT

In a medical information management system, an electronic pen acquires medical information data inputted in handwriting onto a form. A graphic identification unit identifies a graphic area defined by a graphic represented by graphic data contained in the medical information, based on the acquired medical information data. A comment area identification unit identifies a comment area located in a predetermined position relative to the identified graphic area. A comment data identification unit identifies comment data inputted in handwriting to the comment area, among the acquired medical information data. A storage unit stores the acquired medical information data and the identified comment data in a manner that associates the acquired medical information data with the identified comment data.

### CROSS-REFERENCE TO RELATED APPLICATIONS

This application is based upon and claims the benefit of priority from the prior Japanese Patent Applications No. 2005-232343, filed Aug. 10, 2005, and Japanese Patent Application No. 2005-202717, filed Jul. 12, 2005, the entire contents of which are incorporated herein by reference.

### BACKGROUND OF THE INVENTION

1. Field of the Invention

The present invention relates to a medical information management system and a medical information management method and it particularly relates to a medical information management system and a medical information management method in which the medical information inputted in handwriting is handled and managed.

2. Description of the Related Art

In the field of medicine, it has been a general practice to record medical information, such as a doctor's opinion and diagnostic information, in a patient's chart made of paper. In recent years, however, electronization or computerization of patients' charts by use of personal computers has been finding quickly widening use helped by the technological innovation in the medical field. However, since medical information includes figures and other schematic information, it is not easy to input such schematic information through a keyboard or mouse or the like. An example addressing this problem is Reference (1), which proposes a medical information input system in which a server acquires data representing records having been entered in a specific form with an electronic pen and checks the acquired data against the patient. Another example disclosed in Reference (2) proposes a clinical chart management system in which data representing records entered in a specific form with an electronic pen are acquired and entered in a database and any additional recording in the paper form is also entered as additional data.Related Art List

(1) Japanese Patent Application Laid-Open No. 2004-30257.

(2) Japanese Patent Application Laid-Open No. 2004-54375.

References (1) and (2) disclose a technology for acquiring data representing the records entered in a specific form with an electronic pen and entering the acquired data in a database. However, technology for further utilizing such data is not disclosed. Therefore, it is strongly desired that technology be developed for further utilizing electronic information representing medical information.

### SUMMARY OF THE INVENTION

The present invention has been made to solve a problem as described above, and a general purpose thereof is to provide a technology for effectively utilizing electronic information representing medical information which has been inputted in handwriting.

In order to solve the above problems, a medical information management system according to one embodiment of the present invention comprises: a medical information data acquisition unit which acquires medical information data inputted in handwriting; a graphic area identification unit which identifies a graphic area defined by a graphic represented by graphic data contained in the medical information, based on the acquired medical information data; a comment area identification unit which identifies a comment area located in a predetermined position relative to the identified graphic area; a comment data identification unit which identifies comment data inputted in handwriting to the comment area, among the acquired medical information data; and a medical information storage unit which stores the acquired medical information data and the identified comment data in an associated manner.

According to this embodiment, the medical information data representing the medical information inputted in handwriting is associated with the comment data identified from the medical information data. Thus, the comment data can be effectively utilized in a manner that associates it with the medical information data.

Another embodiment of the present invention relates also to a medical information management system. The medical information management system according to this embodiment comprises: a medical information data acquisition unit which acquires medical information data inputted in handwriting; a graphic area storage unit which stores graphic area data indicative of a graphic area defined by a graphic indicated beforehand in an input area where the medical information data are inputted in handwriting; a comment area identification unit which identifies a comment area located in a predetermined position relative to the graphic area indicated by the graphic area data stored in the graphic area storage unit; a comment data identification unit which identifies comment data inputted in handwriting to the comment area, among the medical information data; and a medical information storage unit which stores the acquired medical information data and the identified comment data in an associated manner. According to this embodiment, medical information can be effectively utilized as electronic information even in the case where the medical information is inputted, in handwriting, onto a form on which figures are indicated beforehand.

It is to be noted that any arbitrary combination of the above-described structural components or rearrangement in the form among a method, an apparatus, a system, a computer program, a recording medium storing the computer programs and so forth are all effective as and encompassed by the present embodiments.

### BRIEF DESCRIPTION OF THE DRAWINGS

Embodiments will now be described by way of examples only, with reference to the accompanying drawings which are meant to be exemplary, not limiting.

FIG. 1 illustrates a general structure of a medical information management system according to a first embodiment of the present invention;

FIG. 2 is a flowchart showing processings performed on medical information data by a medical information management system according to a first embodiment of the present invention;

FIG. 3A and FIG. 3B illustrate an example where medical information data inputted onto a form in handwriting are processed in a medical information management system according to a first embodiment of the present invention; and in particular FIG. 3A illustrates medical information inputted manually onto the form and FIG. 3B illustrates medical information and the like displayed on a patient's chart screen;

FIG. 4A illustrates medical information manually inputted onto a form; and FIG. 4B illustrates a state of the medical information processed and displayed on a patient's chart screen by a medical information management system according to a second embodiment of the present invention;

FIG. 5 is a flowchart showing the processings performed on medical information data by a medical information management system according to a third embodiment of the present invention;

FIG. 6A illustrates medical information manually inputted onto a form; and FIG. 6B illustrates a state of the medical information processed and displayed on a patient's chart screen by a medical information management system according to a third embodiment of the present invention;

FIG. 7A illustrates medical information manually inputted onto a form; and FIG. 7B illustrates a state of the medical information processed and displayed on a patient's chart screen by a medical information management system according to a fourth embodiment of the present invention;

FIG. 8 is a flowchart showing processings performed on medical information data by a medical information management system according to a fifth embodiment of the present invention;

FIG. 9 shows an example of form used in a medical information management system according to a fifth embodiment of the present invention;

FIG. 10A illustrates medical information manually inputted onto a form; and FIG. 10B illustrates a state of the medical information processed and displayed on a patient's chart screen by a medical information management system according to a fifth embodiment of the present invention;

FIG. 11 is a flowchart showing processings performed on medical information data by a medical information management system according to a sixth embodiment of the present invention;

FIG. 12A illustrates medical information manually inputted onto a form; and FIG. 12B illustrates a state of the medical information processed and displayed on a patient's chart screen by a medical information management system according to a sixth embodiment of the present invention;

FIG. 13 is a flowchart showing processings performed on medical information data by a medical information management system according to a seventh embodiment of the present invention;

FIG. 14A illustrates medical information manually inputted onto a form; and FIG. 14B illustrates a state of the medical information processed and displayed on a patient's chart screen by a medical information management system according to a seventh embodiment of the present invention;

FIG. 15 illustrates a general structure of an electronic patient's chart system according to an eighth embodiment of the present invention;

FIG. 16 illustrates a general structure of electronic pen and workstation according to an eighth embodiment of the present invention;

FIG. 17 illustrates a general structure of a server according to an eighth embodiment of the present invention;

FIG. 18 illustrates a data processing table stored in a storage unit in an electronic patient's chart system according to an eighth embodiment of the present invention;

FIG. 19 illustrates an inspection request/report form used in an electronic patient's chart system according to an eighth embodiment of the present invention;

FIG. 20 is a flowchart showing a processing by an electronic patient's chart system according to an eighth embodiment of the present invention;

FIG. 21 illustrates a diagnostic information providing form used in an electronic patient's chart system according to a ninth embodiment of the present invention;

FIG. 22 is a flowchart showing a processing by an electronic patient's chart system according to a ninth embodiment of the present invention;

FIG. 23 illustrates an inspection report form used in an electronic patient's chart system according to a tenth embodiment of the present invention;

FIG. 24 is a flowchart showing a processing by an electronic patient's chart system according to a tenth embodiment of the present invention;

FIG. 25 illustrates an inspection report form used in an electronic patient's chart system according to an eleventh embodiment of the present invention;

FIG. 26 is a flowchart showing a processing by an electronic patient's chart system according to an eleventh embodiment of the present invention;

FIG. 27 illustrates an inspection report form used in an electronic patient's chart system according to a twelfth embodiment of the present invention;

FIG. 28 is a flowchart showing a processing by an electronic patient's chart system according to a twelfth embodiment of the present invention;

FIG. 29 illustrates an implementation record card used in an electronic patient's chart system according to a thirteenth embodiment of the present invention;

FIG. 30 is a flowchart showing a processing by an electronic patient's chart system according to a thirteenth embodiment of the present invention;

FIG. 31 illustrates an instruction card used in an electronic patient's chart system according to a fourteenth embodiment of the present invention;

FIG. 32 is a flowchart showing a processing by an electronic patient's chart system according to a fourteenth embodiment of the present invention;

FIG. 33 is a flowchart showing a processing by an electronic patient's chart system according to a fifteenth embodiment of the present invention;

FIG. 34A illustrate a state in which the entry of deletion and addition is made onto an instruction card used in an electronic patient's chart system according to a fifteenth embodiment of the present invention; and FIG. 34B illustrates a state in which the instruction card in FIG. 34A is printed again;

FIG. 35 illustrates an implementation record card used in an electronic patient's chart system according to a sixteenth embodiment of the present invention;

FIG. 36 is a flowchart showing a processing by an electronic patient's chart system according to a sixteenth embodiment of the present invention;

FIG. 37 illustrates a nursing record form used in an electronic patient's chart system according to a seventeenth embodiment of the present invention;

FIG. 38 is a flowchart showing a processing by an electronic patient's chart system according to a seventeenth embodiment of the present invention;

FIG. 39 illustrates a nursing record form used in an electronic patient's chart system according to an eighteenth embodiment of the present invention;

FIG. 40 is a flowchart showing the processing of an electronic patient's chart system according to an eighteenth embodiment of the present invention;

FIG. 41A illustrates a nursing record printed in an electronic patient's chart system according to an eighteenth embodiment of the present invention; and FIG. 41B illustrates a state where an entry is made in the nursing record;

FIG. 42 illustrates a problem list form used in an electronic patient's chart system according to a nineteenth embodiment of the present invention;

FIG. 43 is a flowchart showing a processing by an electronic patient's chart system according to a nineteenth embodiment of the present invention;

FIG. 44 illustrates a preoperative call form used in an electronic patient's chart system according to a twentieth embodiment of the present invention;

FIG. 45 is a flowchart showing a processing by an electronic patient's chart system according to a twentieth embodiment of the present invention;

FIG. 46 illustrates an intraoperative record form used in an electronic patient's chart system according to a twenty-first embodiment of the present invention; and

FIG. 47 is a flowchart showing a processing by an electronic patient's chart system according to a twenty-first embodiment of the present invention.

### DETAILED DESCRIPTION OF THE INVENTION

The invention will now be described by reference to the preferred embodiments. This does not intend to limit the scope of the present invention, but to exemplify the invention.

The description of embodiments of the present invention will be given in detail hereinbelow with reference to drawings.

### First Embodiment

A description of a medical information management system according to the present embodiments of the present invention will be given before a specific description thereof. First, a user manually inputs medical information onto a form 10 using an electronic pen 12. In a medical information management system according to the present embodiments, medical information having been entered onto the form 10 is acquired as medical information data, and a graphic area demarcated by a graphics represented by a graphic data contained in the medical information data on the form is identified. Further, a comment area in a predetermined position relative to the identified graphic area is identified. Of the acquired medical information data, the comment data having been manually inputted to the comment area are identified, and the acquired medical information data, associated with this identified comment data, are stored in a storage unit.

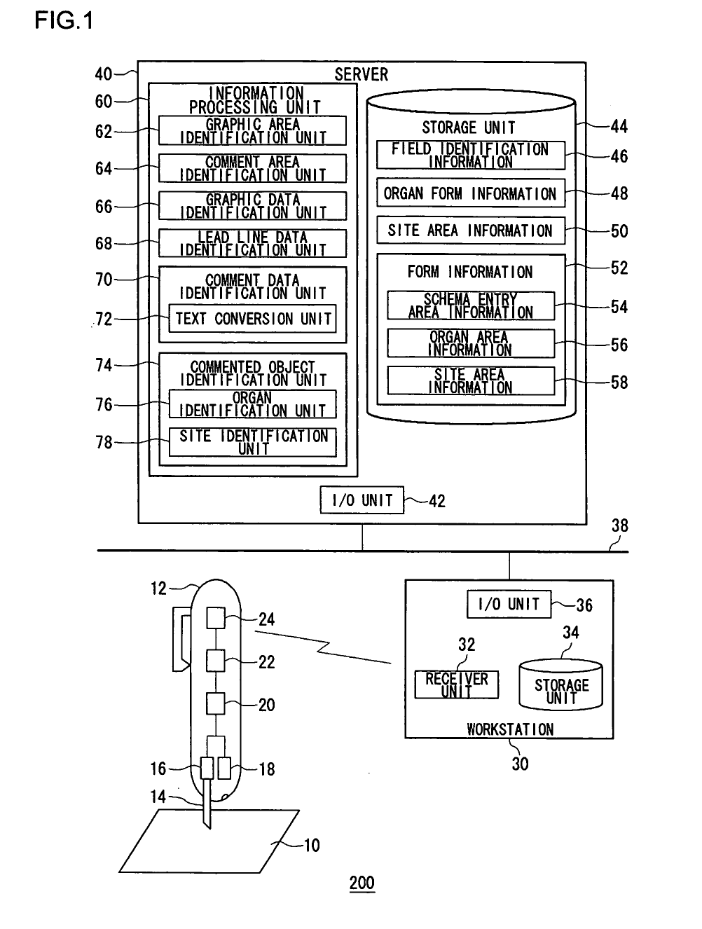

FIG. 1 illustrates a general structure of a medical information management system 200 according to a first embodiment of the present invention. The medical information management system 200 includes an electronic pen 12, a workstation 30, a server 40 and so forth.

In the medical information management system 200, as a user makes a handwritten input onto a form 10 using an electronic pen 12, the electronic pen 12 can acquire the content of entry onto the form 10 successively. Thus, the form 10 functions as an input medium through which medical information data are manually inputted, and the electronic pen 12 functions as a data acquisition unit for acquiring the content of information inputted onto the form 10 as data. This electronic pen 12 includes a pen part 14, a pressure-sensing part 16, a camera part 18, a processing part 20, a storage part 22, and a transmission part 24.

The pen part 14 carries out a recording onto the form 10 by emitting ink as the pen point pressed to the form 10 is moved. The pressure-sensing part 16, which is fixed to the pen part 14, has a piezoelectric element and thereby detects forces and pressures that work on the pen part 14. When an entry onto the form 10 is being made by the pen part 14, the pressure-sensing part 16 detects the handwritten input being made by the electronic pen 12 because the pen part 14 is pressed against the form 10. The camera part 18, using infrared rays, reads the dot pattern printed on the paper surface of the form 10 around the tip of the pen part 14.

The form 10 has a special dot pattern, printed with an ink containing carbon, formed thereon. The area or field where this dot pattern is formed is an input area where a user inputs medical information using an electronic pen 12. Medical information meant here is information entered onto the form by a physician or the like, which includes, for instance, a patient's name, attending physician's name, date of diagnosis, schema of an internal organ or affected part, the physician's opinion, diagnostic results and lead lines, which are all to be recorded on a patient's chart or the like, as well as reference information including reference numbers.

Each of the dots of a dot pattern printed in the input area is arranged two-dimensionally in such a manner that its location can be identified. As a manual input onto this form 10 is initiated by the electronic pen 12, the pressure-sensing part 16 first detects the manual input being made by the electronic pen 12. While the manual input is being detected by the pressure-sensing part 16, the camera part 18 reads the dot pattern on the form 10. In this manner, the electronic pen 12 can acquire the locus of the tip of the pen part 14 during the manual input and thereby acquire the content of handwritten entry onto the form 10 as data. Accordingly, when medical information, such as schema, a physician's opinion or diagnostic results, is hand-written onto the form 10 with an electronic pen 12, the electronic pen 12 can acquire the manually inputted medical information as medical information data representing it.

It is to be noted here that each form 10 has its own form ID. The electronic pen 12 can acquire this form ID by reading the dot pattern. Hence, even when some additional entry is made onto the same form 10, data representing the content of previous entry can be easily identified and the additional data can be easily associated with the previous data. As will be discussed later, the form ID can also be used to identify the form.

The processing part 20 performs processings such as data compression and addition of the ID of the electronic pen 12 for the data read by the camera part 18. The storage part 22 stores data having been processed by the processing part 20. The transmission part 24 transmits information stored in the storage part 22 to the outside wirelessly by Bluetooth (registered trademark). The electronic pen 12 has a USB (Universal Serial Bus) interface (not shown). Accordingly, the electronic pen 12 can also output data through wire to the outside using a USB cable.

A workstation 30 is placed in each of the relevant divisions, which may comprise an inspection system of endoscopy, pathology, etc., a ward system containing a plurality of hospital wards, a support system of medical accounting, pharmacology, etc., and the like, and is connected with the other workstations via a network 38. Also connected to the network 38 is a server 40, which will be described later, and the workstations 30 of the divisions as mentioned above can transmit data to and receive data from the server 40.

The workstation 30 includes a receiver unit 32, a storage unit 34, an input/output (I/O) unit 36 and so forth. The receiver unit 32 receives data transmitted from the electronic pen 12. The storage unit 34 stores the data received by the receiver unit 32. The input/output unit 36 carries out data transmission to and reception from the server 40 or workstations 30 of the other divisions via the network 38. Data received from the electronic pen 12 and stored in the storage unit 34 are outputted to the server 40 by the input/output unit 36 by way of the network 38. In this manner, the workstation 30 performs the function of a data relay unit by receiving data from the electronic pen 12 and outputting data to the server 40.

The server 40 includes an input/output unit 42, a storage unit 44 and an information processing unit 60. The input/output unit 42 performs data transmission to and reception from workstations 30 of the respective divisions via the network 38. Data obtained by the electronic pen 12 are inputted to the server 40 by this input/output unit 42 through the workstation 30 and the network 38.

The storage unit 44 stores data inputted from the workstation 30 via the input/output unit 42. Thus the storage unit 44 functions as a medical information storage unit for storing medical information data. The storage unit 44 not only stores data inputted from the workstation 30 as mentioned above, but also stores area identification information 46, organ form information 48, site area information 50, and form information 52 in advance.

The area identification information 46 includes relative position information for specifying the position of a comment area relative to a graphic area of medical information data to be described later. The relative position information may, for instance, contain information indicating that the comment area is inside the graphic area or information indicating that the comment area is outside the graphic area. Where there are a plurality of relative position information, one or more relative position information may be set as default. A user may select and set one of such relative position information, using a keyboard, mouse, or the like; that is, the user may, for instance, select the inside or the outside of a graphic area for a comment area and set it accordingly. A comment area identification unit 64, which will be described later, can identify a comment area according to the setting of the area identification information 46.

The organ form information 48 includes information on the forms of a variety of organs and the names of those organs. Accordingly, the organ form information 48 can be used as graphic area data representing a graphic area of an organ. And the above-mentioned organ name information can be used as graphic area identification data for identifying the area of a figure representing an organ. An organ identification unit 76, to be described later, can use this organ form information 48 to determine which of the organs the schema, which is the manually inputted graphic information, represents.

The site area information 50 includes information on the areas of sites in a variety of organs and the names of those sites. Accordingly, the site area information 50 can be used as partial area data representing the area of a site. And the above-mentioned site name information can be used as partial area identification data for identifying the area of a site. By identifying an area representing a site, a site, which is the position of interest, can be identified from a physician's opinion, a figure of an affected part, a lead line, or the like recorded within an organ, when a manually inputted graphic schema is determined to represent an organ, for instance, by the use of organ form information 48.

A physician or the like, when he/she inputs medical information in handwriting, may sometimes use a form which is provided with entry spaces for entry of various medical information or with a graphic form of an organ. In preparation for cases like this, the storage unit 44 contains form information 52, which is used to identify the areas of entry spaces or a graphic of an organ on the form. The form information 52 includes schema entry area information 54, organ area information 56, and site area information 58.

When a physician or the like inputs medical information in handwriting, he/she may sometimes use a form which is provided with a patient's name space, a physician's name space, a diagnosis date space and the like for entry of their respective information and, in addition thereto, with a schema entry space for entry of a schema. For effective use of medical information, it is preferable that a distinction be made between comments related to a schema and information on names, dates and the like. Accordingly, the storage unit 44 contains schema entry area information 54 for identifying the entry area of a schema, and the information processing unit 60 can identify the area for entry of a schema by using this schema entry area information 54.

Also, when a physician or the like inputs medical information in handwriting, he/she may sometimes use a form, which is, for instance, a form 10 provided already with a graphic or figure representing an organ. In such a case, too, it is preferable for effective use of medical information that there is a facility for identifying which of the organs a comment, such as a physician's opinion, relates to. In this regard, the storage unit 44 stores organ area information 56 that can be used as graphic area data representing a graphic area demarcated by a graphic already shown in the input area. The organ area information 56 includes organ name information, which can indicate the name of the organ presented on the form 10. This organ name information can be used as graphic area identification data for identifying a graphic area. Included in the organ area information 56 is information that indicates the area of the organ presented on the form. Hence, when a physician or the like manually inputs medical information on such a form using an electronic pen 12, an organ identification unit 76 can identify the area of the organ presented on the form by use of the organ area information 56.

Also, for effective use of medical information, it is preferable that there is a facility for identifying which of the sites of an organ a comment, such as a physician's opinion, relates to, even when a form as described above is used. In this regard, the storage unit 44 stores site area information 58 that can be used as partial area data representing a partial area included in the graphic area. The site area information 58 includes site name information, which can indicate the name of the site presented on the form 10. This site name information can be used as partial area identification data for identifying a partial area. Included in the site area information 58 is information that indicates the respective sites of the organ presented on the form. Hence, when a physician or the like manually inputs medical information on such a form using an electronic pen 12, a site identification unit 78 can identify which of the organs or the sites thereof a comment, such as a physician's opinion, relates to.

The information processing unit 60 carries out predefined processings on the medical information data which has been acquired by an electronic pen 12 and inputted through a network 38. The information processing unit 60 includes a graphic area identification unit 62, a comment area identification unit 64, a graphic data identification unit 66, a lead line data identification unit 68, a comment data identification unit 70, and a commented object identification unit 74.

The graphic data identification unit 66 identifies graphic data from the inputted medical information data. The graphic data identification unit 66 identifies graphic data in such a manner that a manually-inputted continuous line, for instance, is recognized as a line constituting a graphic, and not a line constituting a character, if the length thereof is equal to or larger than a predetermined value. The graphic area identification unit 62 identifies a graphic area demarcated by a graphic, using the graphic data identified by the graphic data identification unit 66. The comment area identification unit 64 identifies a comment area, using the identified graphic area and the above-mentioned area identification information 46. Thereby, the area where comment data resides can be identified.

The lead line data identification unit 68 identifies lead line data from among medical information data. For example, the lead line data identification unit 68 identifies, as lead line data, medical information data which represents a line having a length equal to or longer than a predetermined length and with one end thereof located in a graphic area and the other end thereof in a comment area. Also, for example, the lead line data identification unit 68 identifies, as lead line data, data which represents a line having a length equal to or longer than a predetermined length and with one end thereof located closer than a predetermined distance to medical information data in a comment area. Also, for example, the lead line data identification unit 68 identifies, as lead line data, medical information data which represents a line having a length equal to or longer than a predetermined length and with one end thereof located in a graphic area and the other end thereof outside the graphic area.

The comment data identification unit 70 identifies comment data by identifying data located in the comment area which has been identified by the comment area identification unit 64 from among the medical information data. The comment data identification unit 70 includes a text conversion unit 72, which converts image data or line data into text data. The comment data identification unit 70 has the identified comment data converted to text information by the text conversion unit 72. This improves the usability of comment data.

The commented object identification unit 74 identifies which of the organs or the sites thereof a comment, such as a physician's opinion or the results of diagnosis, relates to. The commented object identification unit 74 includes an organ identification unit 76 and a site identification unit 78. The organ identification unit 76 identifies an organ meant by comment data by referring to organ form information 48 stored in the storage unit 44, organ area information 56 in the form information 52 and the like. The site identification unit 78 identifies a site of an organ meant by a comment data by referring to site area information 50 stored in the storage unit 44, site area information 58 and the like.

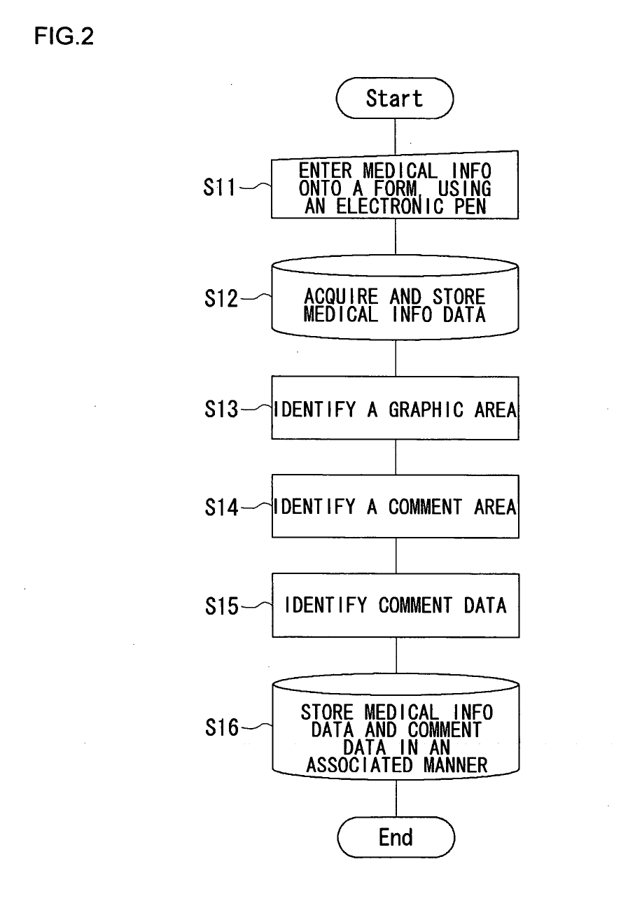

FIG. 2 is a flowchart showing processings performed on medical information data by a medical information management system 200 according to a first embodiment of the present invention.

Firstly, a user manually inputs medical information 100 onto a form 10, using an electronic pen 12 (S11). As the medical information is manually inputted onto the form 10, a camera part 18 reads the dot pattern on the form 10 and stores it as electronic data in a storage part 22. As a result, the electronic pen 12 acquires the medical information manually inputted onto the form 10 as medical information data. The medical information data thus acquired are transmitted to a workstation 30 at predetermined intervals or at the input of manual input completion, before they are inputted to a server 40 via the workstation 30 and a network 38. The medical information data inputted to the server 40 are stored in a storage unit 44 (S12). Note that “at the input of manual input completion” meant here is, for instance, when a check indicating the end of writing is entered by the electronic pen 12 in a predetermined area of the form 10.

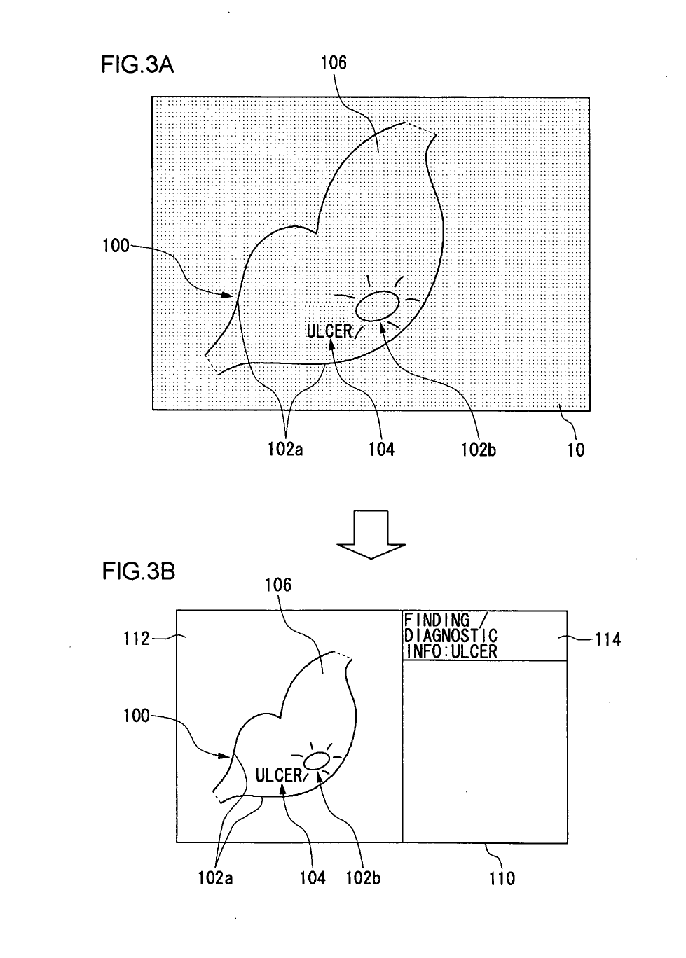

Let us suppose, for instance, that a physician has manually inputted medical information 100 as shown in FIG. 3A onto a form 10 with an electronic pen 12. The medical information 100 includes a schema 102 and a comment 104, such as the physician's opinion and the results of diagnosis. In FIG. 3A, the schema 102 includes an organ graphic 102a and an affected site graphic 102b. In FIG. 3A, the organ graphic 102a represents a cross-sectional drawing of a stomach, and the affected site graphic 102b represents an affected site drawing of an ulcer having formed in the stomach. Also entered in the organ graphic 102a is a comment 104, which is the physician's opinion indicating “ulcer” in characters.

The electronic pen 12 reads the dot pattern printed on the form 10 when the medical information 100 is being inputted onto the form 10 in handwriting. In this manner, the electronic pen 12 acquires the medical information 100 including the organ graphic 102a, the affected site graphic 102b and the comment 104 as the medical information data, which is electronic information. The medical information data thus acquired are transmitted to the workstation 30, before they are inputted to the server 40 via the workstation 30 and the network 38 and then stored in a storage unit 44.

Upon completion of the acquisition and storage of medical information data, the graphic data identification unit 66 first identifies graphic data from the acquired medical information data. Based on the thus identified graphic data, the graphic area identification unit 62 identifies a graphic area demarcated by the identified graphic in the medical information represented by the acquired medical information data (S13). In the example of the medical information 100 shown in FIG. 3A, the graphic data identification unit 66 identifies as graphic data the medical information data of two lines representing the organ graphic 102a and the medical information data representing the affected site graphic 102b entered between the two lines by taking the lengths of the lines inputted onto the form 10 and the like into consideration. Note by the way that site graphic data representing a plurality of site graphics may also be stored in the storage unit 44. The graphic data identification unit 66 may search for medical information data similar to the affected site graphic represented by the affected site graphic data and, if there is a similar medical information data, may identify the medical information data as graphic data representing the affected site graphic. In the example of the medical information 100 shown in FIG. 3A, if the affected site graphic 102b is similar to an affected site graphic represented by an affected site graphic data stored in the storage unit 44, then the graphic data identification unit 66 identifies the affected site graphic 102b as the graphic data representing the affected site.

When a graphic area is not closed by a graphic identified by the graphic data identification unit 66, the graphic area identification unit 62 identifies the graphic area by interpolating the open portion thereof with an interpolating line. In the example of the medical information 100 shown in FIG. 3A, the organ graphic 102a, which consists of two lines delineating a cross section of a stomach, has the lines separated at the entrance and the exit of the stomach. Therefore, the graphic area identification unit 62 adds interpolating lines connecting the ends of the two lines in order to identify the graphic area. In this manner, the organ graphic 102a can be made a closed area, and thus the graphic area can be identified.

Also, when there are a plurality of areas demarcated by a graphic represented by identified graphic data, the graphic area identification unit 62 identifies all of the areas demarcated by the graphic represented by the identified graphic data as graphic areas. Further, if a plurality of areas overlap each other, the plurality of overlapping areas are identified as a single graphic area. Also, when a larger area includes a smaller area within it, the larger area is identified as the graphic area. In the example of the medical information 100 shown in FIG. 3A, there are an area demarcated by the organ graphic 102a consisting of two lines delineating a cross section of a stomach and an area demarcated by the affected site graphic 102b containing a graphic representing the site of an ulcer. In the example of FIG. 3A, the affected site graphic 102b is contained in the organ graphic 102a, so that the graphic area identification unit 62 identifies the organ graphic 102a as the graphic area 106.

Upon identification of a graphic area, the comment area identification unit 64 identifies a comment area (S14). The comment area identification unit 64 identifies the position of a comment area relative to a graphic area based on the settings of area identification information 46 stored in the storage unit 44. According to the first embodiment, relative position information, in which a comment area is inside a graphic area, is set in the area identification information 46. Accordingly, the comment area identification unit 64 identifies the inside of the graphic area as the comment area. In the example of the medical information 100 shown in FIG. 3A, the graphic area 106, as it is, is identified as the comment area.

Upon identification of a comment area, the comment data identification unit 70 identifies as comment data the medical information data positioned in the area identified as the comment area (S15). In so doing, comment data can be extracted efficiently by excluding the medical information data outside the comment area from the comment data. The comment data identification unit 70 has a text conversion unit 72 convert the identified comment data into text information. In the example of the medical information 100 shown in FIG. 3A, an organ identification unit 76 identifies as comment data the medical information data representing the comment 104 consisting of the characters “ulcer”, which are the medical information 100 entered inside the graphic area 106. The medical information data consisting of the characters “ulcer” is converted into text information by the text conversion unit 72.

Upon identification of a comment data, the medical information data and the comment data converted into text information are associated with each other and stored in the storage unit 44 (S16). As a result, it is now possible to effectively use the medical information data representing the manually inputted medical information.

The medical information data and the comment data, which have been associated with each other and stored in the storage unit 44, can be browsed from a server 40 or a workstation 30 located at each division. In such a case, a patient's chart screen 110 as illustrated in FIG. 3B is displayed on a display (not shown).

The patient's chart screen 110 is comprised of a medical information screen 112, a comment information screen 114, and so forth. Displayed on the medical information screen 112 is medical information 100 which is represented by the medical information data inputted to the server 40. On the medical information screen 112, all of the medical information 100 represented by the inputted medical information data is displayed. For example, the characters “ulcer”, which is an opinion or finding manually inputted by a physician or the like, is also displayed on the medical information screen 112 in the mode of manual input used. In this manner, the same information as medical information 100 that is inputted onto a form 10 in handwriting can be displayed on the medical information screen 112.

When the medical information 100 is displayed on the medical information screen 112, the comment data stored in association with the medical information 100 is displayed on the comment information screen 114. At this time, a manually-inputted comment 104 is displayed on the comment information screen 114 as text information after text conversion. For example, the characters “ulcer”, which is an opinion manually inputted by a physician or the like, is converted into text and displayed on the comment information screen 114. As described above, a comment, such as a physician's opinion or the results of diagnosis, is first displayed on the comment information screen 114 in distinction from the schema, so that it is possible to check the comment, such as a physician's opinion or the results of diagnosis, simply by looking at the comment information screen 114. In the example of FIG. 3B, a person who takes a look at the medical information screen 112, can see that the physician's opinion is “ulcer” by looking at the comment information screen 114. The arrangement like this allows a quick check of a comment, thus providing an efficient medical environment to physicians and the like. Also, the comment information, which comes converted to text information, provides an easy-to-understand display of comment to those who see the comment information screen 114. Moreover, the text information after text conversion allows not only the enlargement of characters of comment information but also the change in font or color thereof quite easily, further improving the easy-to-understand display of comment.

### Second Embodiment

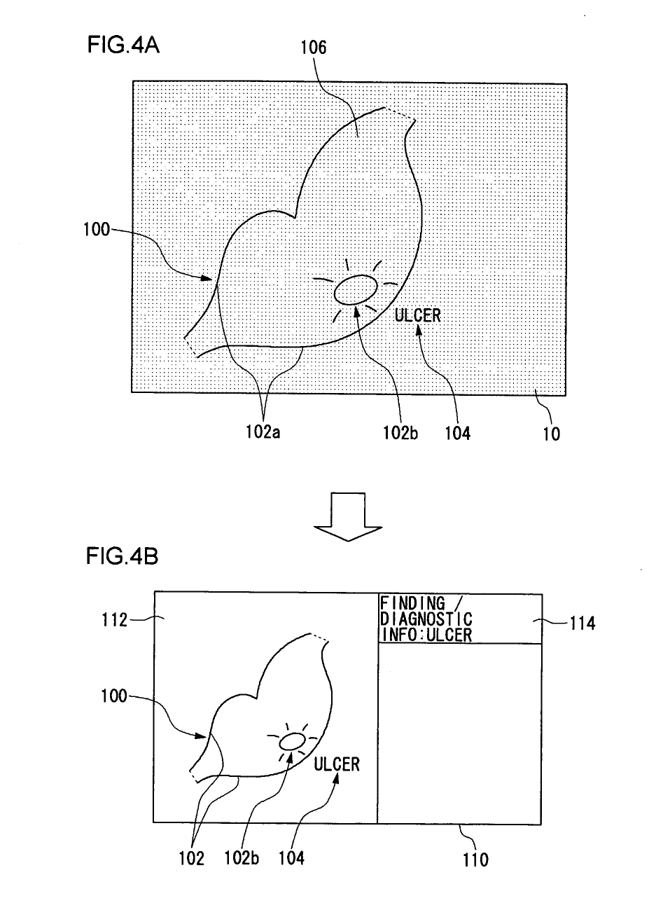

FIG. 4A illustrates medical information 100 manually inputted onto a form 10, and FIG. 4B illustrates a state of the medical information 100 processed and displayed on a patient's chart screen 110 by a medical information management system 200 according to a second embodiment of the present invention. Hereinbelow, descriptions identical to those of the first embodiment will be omitted.

According to this second embodiment, a relative position information indicating that the comment area is outside the graphic area is set in area identification information 46. Accordingly, in the example of the medical information 100 shown in FIG. 4A, a graphic area identification unit 62 identifies the area demarcated by an organ graphic 102a as a graphic area 106 as in the first embodiment. Consequently, a comment area identification unit 64 identifies the outside of the organ graphic 102a as a comment area. In FIG. 4A, a comment 104 is located outside the organ graphic 102a. Accordingly, a comment data identification unit 70 identifies the medical information data representing the comment 104 as comment data. The comment data identification unit 70 carries out a text conversion on the identified comment data.

The medical information data is associated with the comment data converted into text information before they are stored in a storage unit 44. Thus, as shown in FIG. 4B, the medical information 100 can be displayed on a patient's chart screen 110, and the comment data converted into text information can be displayed on a comment information screen 114, for a browsing from a server 40 or a workstation 30 located at each division.

As described above, a comment, such as a physician's opinion or the results of diagnosis, is first displayed on the comment information screen 114 in distinction from the schema, so that it is possible to check the comment, such as a physician's opinion or the results of diagnosis, simply by looking at the comment information screen 114. In the example of FIG. 4B, a person who takes a look at the medical information screen 112, can figure out that the physician's opinion is “ulcer” by looking at the comment information screen 114. The arrangement like this allows a quick check of a comment, thus providing an efficient medical environment to physicians and the like. Also, the comment information, which comes converted to text information, provides an easy-to-understand display of comment to those who see the comment information screen 114. Moreover, the text information after text conversion allows not only the enlargement of characters of comment information but also the change in font or color thereof quite easily, further improving the easy-to-understand display of comment. As a result, it is now possible to effectively use the medical information data representing the manually inputted medical information.

### Third Embodiment

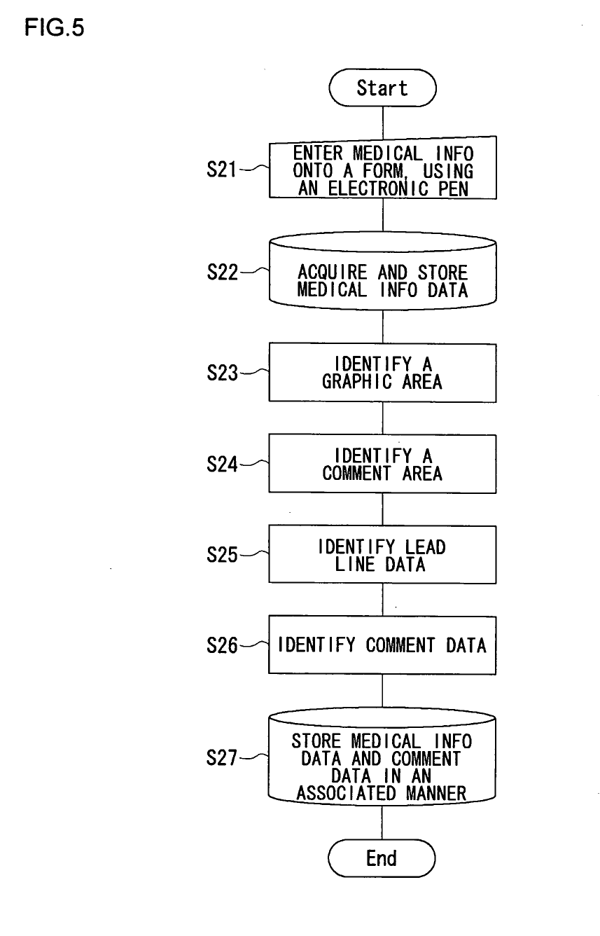

FIG. 5 is a flowchart showing the processings performed on medical information data by a medical information management system 200 according to a third embodiment of the present invention. The description of S21 to S23, which is the same as that of S11 to S13 in FIG. 3, will be omitted.

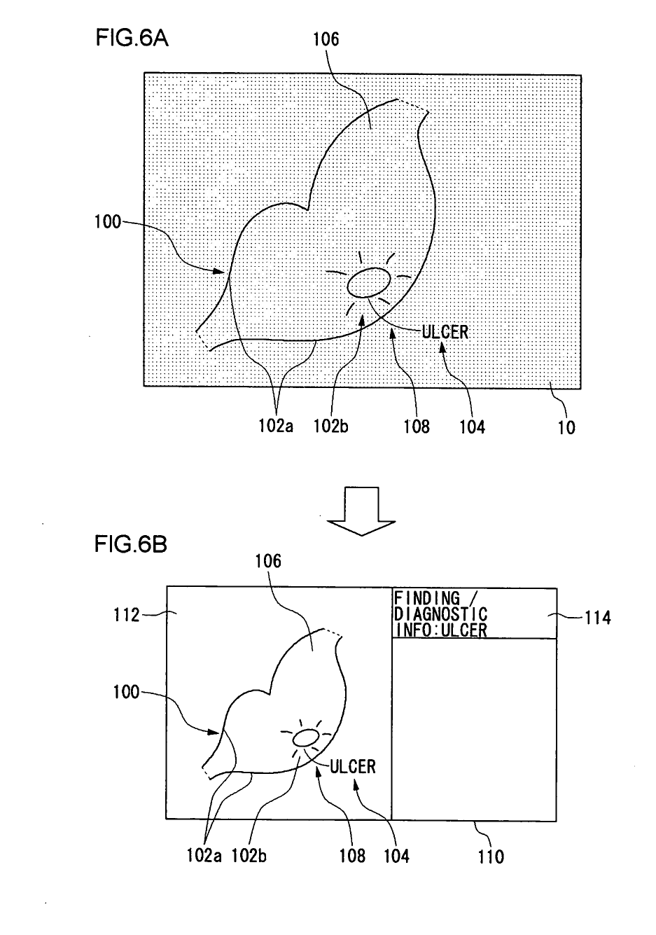

Upon identification of a graphic area, a comment area identification unit 64 identifies a comment area (S24). In the present embodiment, a relative position information indicating that a comment area is outside a graphic area is set in the area identification information 46. Accordingly, the comment area identification unit 64 identifies the outside of the identified graphic area as the comment area. In the example of the medical information 100 shown in FIG. 6A, a graphic area identification unit 62 identifies the area demarcated by an organ graphic 102a as a graphic area 106 in the same way as in the first embodiment. Accordingly, the comment area identification unit 64 identifies the outside of the organ graphic 102a as the comment area.

Upon identification of a comment area, a lead line data identification unit 68 identifies lead line data (S25). The lead line data identification unit 68 identifies, as lead line data, such data regarding a line which has a length equal to or greater than a predetermined length and the ends of which are located in a graphic area and a comment area, respectively. In the example of FIG. 6A, the lead line 108 has one end thereof located inside the graphic area 106 and the other end thereof located in the comment area outside the graphic area 106. Accordingly, the lead line data identification unit 68 identifies the lead line 108 as the lead line data.

Upon identification of lead line data, a comment data identification unit 70 identifies comment data (S26). The comment data identification unit 70 identifies, as comment data, medical information data representing medical information which is located in the comment area and at the same time closer than or equal to a predetermined distance to one end of the lead line represented by the identified lead line data. In the example of FIG. 6A, a comment 104 is located outside the organ graphic 102a and at the same time closer than or equal to a predetermined distance to one end of the lead line. Accordingly, the comment data identification unit 70 identifies the medical information data representing the comment 104 as the comment data. A text conversion unit 72 carries out a text conversion on the identified comment data.

The medical information data and the comment data converted into text information are associated with each other and stored in a storage unit 44 (S27). Thus, as shown in FIG. 6B, the medical information 100 can be displayed on a patient's chart screen 110, and the comment data converted into text information on a comment information screen 114, for a browsing from any of workstations 30 located at the respective divisions. In the example of FIG. 6B, comment data in the characters “ulcer” is displayed as text information on the comment information screen 114.

As described above, a comment, such as a physician's opinion or the results of diagnosis, is first displayed on the comment information screen 114 in distinction from the schema, so that it is possible to check the comment, such as a physician's opinion or the results of diagnosis, simply by looking at the comment information screen 114. In the example of FIG. 6B, a person who takes a look at the medical information screen 112 can see that the physician's finding is “ulcer” by looking at the comment information screen 114. The arrangement like this allows a quick check of a comment, thus providing an efficient medical environment to physicians and the like. Also, the comment information, which comes converted to text information, provides an easy-to-understand display of comment to those who see the comment information screen 114. Moreover, the text information after text conversion allows not only the enlargement of characters of comment information but also the change in font or color thereof quite easily, further improving the easy-to-understand display of comment. As a result, even when the manually inputted medical information includes a lead line, it is possible to effectively use the medical information data representing this medical information.

### Fourth Embodiment

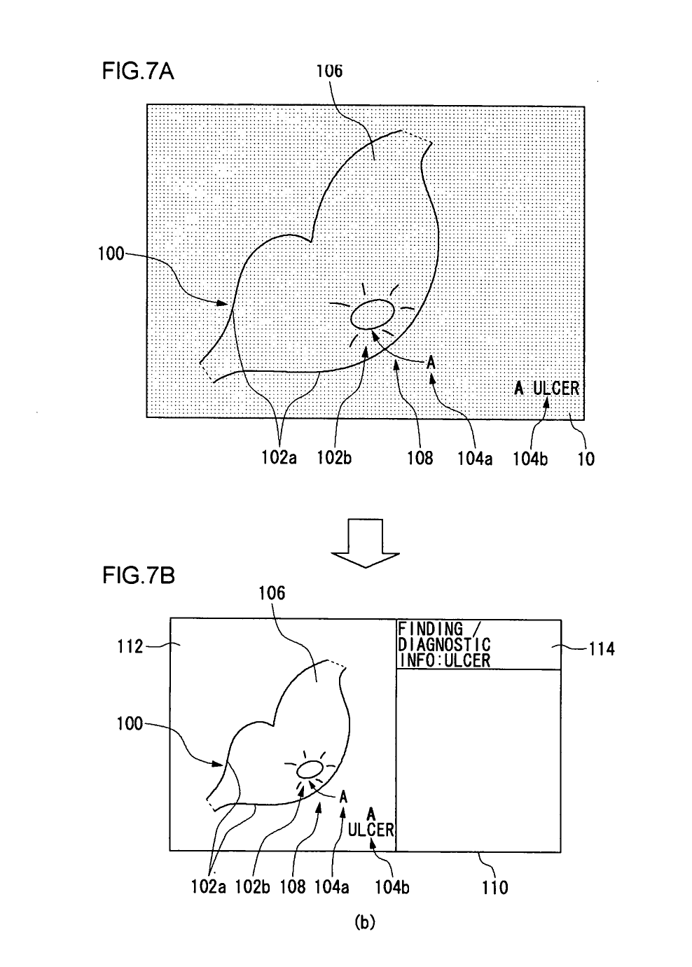

FIG. 7A illustrates medical information 100 manually inputted onto a form 10, and FIG. 7B illustrates a state of the medical information 100 processed and displayed on a patient's chart screen 110 by a medical information management system 200 according to a fourth embodiment of the present invention. The description of parts similar to those in the above-described embodiments will be omitted.

According to this fourth embodiment, relative position information indicating that the comment area is outside the graphic area is set in area identification information 46. In the example of the medical information 100 shown in FIG. 7A, a graphic area identification unit 62 identifies the area demarcated by an organ graphic 102a as a graphic area 106. Consequently, a comment area identification unit 64 identifies the outside of the organ graphic 102a as a comment area.

A lead line data identification unit 68 identifies lead line data for a lead line 108 from among medical information data in the same way as in the third embodiment. A comment data identification unit 70 first identifies, as citation information, the medical information located closer than or equal to a predetermined distance to an end of the lead line. At this point, a text conversion unit 72 converts all of the medical information data located in the comment area including citation information into text information. In this processing, a search is made for medical information data which has the same text information as the text information of citation information at the beginning of a series of text information. Where there exists such medical information data, what is left by excluding the medical information data representing the medical information located at the end of the lead line from such medical information data is identified as comment data.

In the example of FIG. 7A, there is an entry of medical information in character “A” located closer than or equal to a predetermined distance to one end of a lead line 108. Accordingly, the comment data identification unit 70 identifies the character “A” as citation information 104a. The text conversion unit 72 converts all of these medical information data into text information and searches for medical information data having character “A” at the beginning thereof. Here, present in the comment area is an entry of medical information in characters “A ULCER”. Since “A ULCER” has the character “A” at the beginning thereof, the comment data identification unit 70 identifies “ULCER”, which is “A” excluded from the medical information data, namely, “A ULCER”, as the comment data.

The comment data identification unit 70 carries out a text conversion on the identified comment data. A storage unit 44 stores the medical information data and the comment data converted into text information after associating them with each other. Thus, as shown in FIG. 7B, the medical information 100 can be displayed on a patient's chart screen 110, and the comment data converted into text information can be displayed on a comment information screen 114, when a user browses the medical information data, using a workstation 30 belonging to any of the divisions. In the example of FIG. 7B, comment data in the characters “ulcer” is displayed as text information on the comment information screen 114.

As described above, a comment, such as a physician's opinion or the results of diagnosis, is first displayed on the comment information screen 114 in distinction from the schema, so that it is possible to check the comment, such as a physician's opinion or the results of diagnosis, simply by looking at the comment information screen 114. In the example of FIG. 7B, a person who takes a look at the medical information screen 112 can see that the physician's opinion is “ulcer” by looking at the comment information screen 114. The arrangement like this allows a quick check of a comment, thus providing an efficient medical environment to physicians and the like. Also, the comment information, which comes converted to text information, provides an easy-to-understand display of comment to those who see the comment information screen 114. Moreover, the text information after text conversion allows not only the enlargement of characters of comment information but also the change in font or color thereof quite easily, further improving the easy-to-understand display of comment. As a result, even when the manually inputted medical information includes a lead line and also a cited or referenced comment, it is possible to effectively use the medical information data representing the medical information.

### Fifth Embodiment

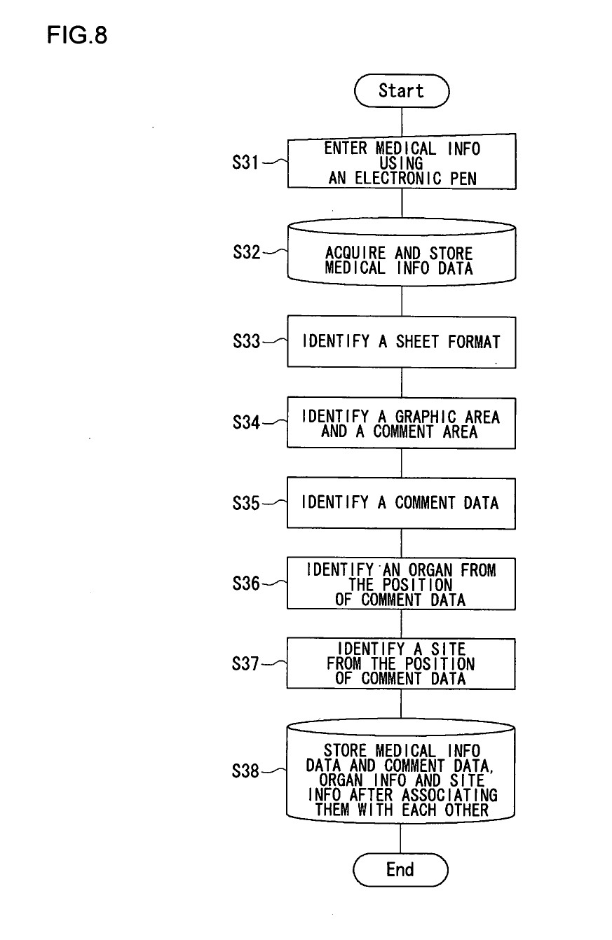

FIG. 8 is a flowchart showing processings performed on medical information data by a medical information management system 200 according to a fifth embodiment of the present invention. The description of parts similar to those in the above-described embodiments will be omitted.

Firstly, a user manually inputs medical information 100 onto a form 10, using an electronic pen 12 (S31). As a result, the electronic pen 12 acquires the medical information manually inputted onto the form 10 as medical information data. The medical information data and form ID thus acquired are inputted to a server 40 via a workstation 30 and a network 38. The medical information data inputted to the server 40 are stored in a storage unit 44 (S32).

The storage unit 44 stores a reference table in which form IDs are associated with forms showing specific organ graphics. As the medical information data acquired by the electronic pen 12 is stored in the storage unit 44, a graphic area identification unit 62 first identifies a form from the form ID and the reference table (S33). Stored as form information 52 in the storage unit 44 are schema entry area information 54, organ area information 56 and site area information 58 for a variety of forms.

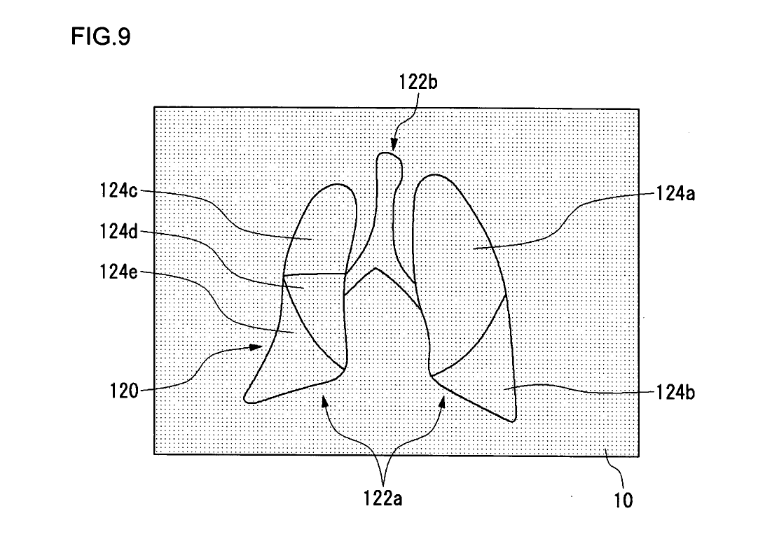

According to the present embodiment, medical information can be entered onto a form already printed with a print graphic 120 such as illustrated in FIG. 9. In the example of this form, the print graphic 120 includes the lungs and bronchus of a human being. From this figure of the lungs, a first organ area 122a, which is the organ area of the lungs, is demarcated. Also, from this figure of the bronchus, a second organ area 122b, which is the organ area of the bronchus, is demarcated. In the example of this form, the first organ area 122a, which is the organ area of the lungs, is further divided into a plurality of areas. In the example of this form, the first organ area 122a is divided into a first site area 124a representing the upper lobe of the left lung, a second site area 124b representing the lower lobe of the left lung, a third site area 124c representing the upper lobe of the right lung, a fourth site area 124d representing the middle lobe of the right lung, and a fifth site area 124e representing the lower lobe of the right lung. The organ area information 56 of the form information 52 includes information on the area of the first organ area 122a, which is the area of the lungs, and information on the area of the second organ area 122b, which is the area of the bronchus.

Upon identification of a form, a comment data identification unit 70 identifies a graphic area by acquiring the organ area information 56 corresponding to the form from the form information 52 stored in the storage unit 44. According to this embodiment, a relative position information indicating that the comment area is inside the graphic area is set in area identification information 46. Consequently, a comment area identification unit 64 identifies the inside of the identified graphic area as a comment area (S34).

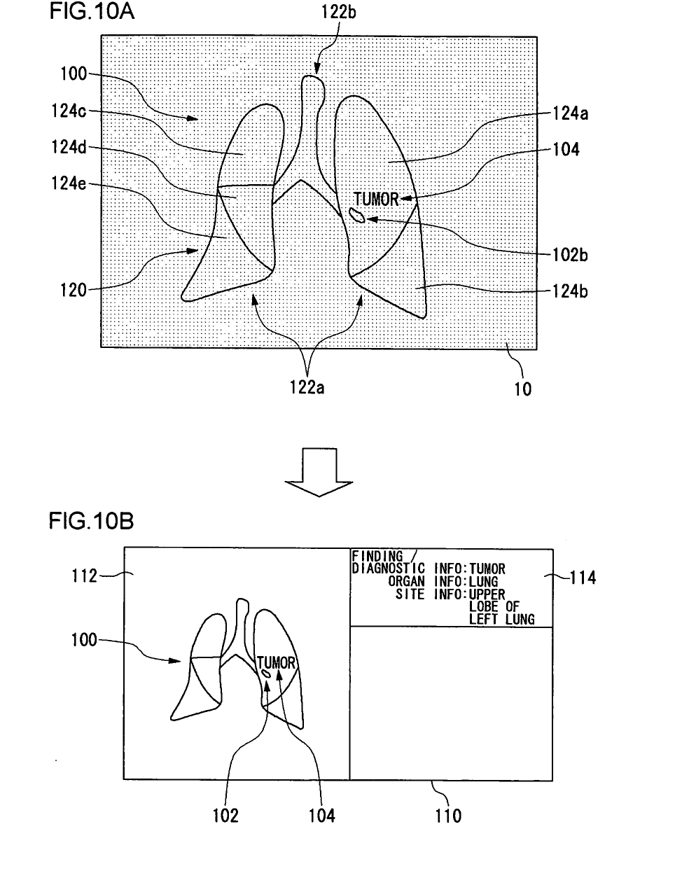

Upon identification of a comment area, the comment data identification unit 70 identifies the medical information data positioned in the comment area as comment data (S35). The identified comment data is converted into text information by a text conversion unit 72. In the example of FIG. 10A, there is an entry of a comment in handwritten characters “ulcer” in the first organ area 122a, which is the comment area. Accordingly, the comment data identification unit 70 identifies the medical information data representing the comment 10 as the comment data. The medical information data representing the identified comment 104 is converted into text information “ulcer” by the text conversion unit 72.

Upon identification of the comment data, an organ identification unit 76 identifies an organ in which the comment is entered from the position of the comment data (S36). The organ identification unit 76 identifies the organ in which the comment is entered by determining which of the organ areas of organs the identified comment data is located in, from the areas of organs contained in organ area information 56 and the position of the identified comment data. In the example of FIG. 10A, the comment 104 is located in the first organ area 122a, which represents the area of the lungs. Accordingly, the organ identification unit 76 identifies the organ information of the medical information data representing the comment 104 as “lungs”.

A site identification unit 78 identifies a site from the position of comment data (S37). The site identification unit 78 identifies the site of an organ in which the comment is entered by determining which of the site areas of sites of organs the identified comment data is located at, from the areas of sites of organs contained in site area information 58 and the position of the identified comment data. In the example of FIG. 10A, the comment 104 is located in the first site area 124a, which represents the “upper lobe of the left lung”. Accordingly, the site identification unit 78 identifies the site information of the medical information data representing the comment 104 as “upper lobe of left lung”.

Upon identification of an organ and a site in the organ from the position of comment data, the storage unit 44 stores the medical information data and the comment data, organ information and site information after associating them with each other (S38). Thus, as shown in FIG. 10B, the medical information 100 can be displayed on a patient's chart screen 110, and the comment data converted into text information, the organ information and the site information can be displayed on a comment information screen 114, when these pieces of information are to be browsed from a server 40 or from a workstation 30 belonging to any of the divisions. In the example of FIG. 10B, comment data in characters “tumor”, organ information in characters “lungs”, and site information in characters “upper lobe of left lung” are displayed as text information.

As described above, a comment, such as a physician's opinion or the results of diagnosis, is first displayed on the comment information screen 114 in distinction from the schema, so that it is possible to check the comment, such as a physician's opinion or the results of diagnosis, simply by looking at the comment information screen 114. The arrangement like this allows a quick check of a comment, thus providing an efficient medical environment to physicians and the like. Also, the comment information, which comes converted to text information, provides an easy-to-understand display of comment to those who see the comment information screen 114. Moreover, the text information after text conversion allows not only the enlargement of characters of comment information but also the change in font or color thereof quite easily, further improving the easy-to-understand display of comment. Also, since organ information and site information related to the comment are displayed, a user can see the comment related to an organ and an affected site of the organ by simply looking at the comment information screen 114. As a result, details of medical information 100 can be grasped by seeing the comment information screen 114. In the example of FIG. 10B, a user who browses the medical information screen 112 can grasp the results of diagnosis that there is a tumor in the upper lobe of the left lung, by seeing the comment information screen 114. In this manner, it is possible to effectively use the medical information data representing the medical information manually inputted using a form 10 already printed with a figure of an organ.

### Sixth Embodiment

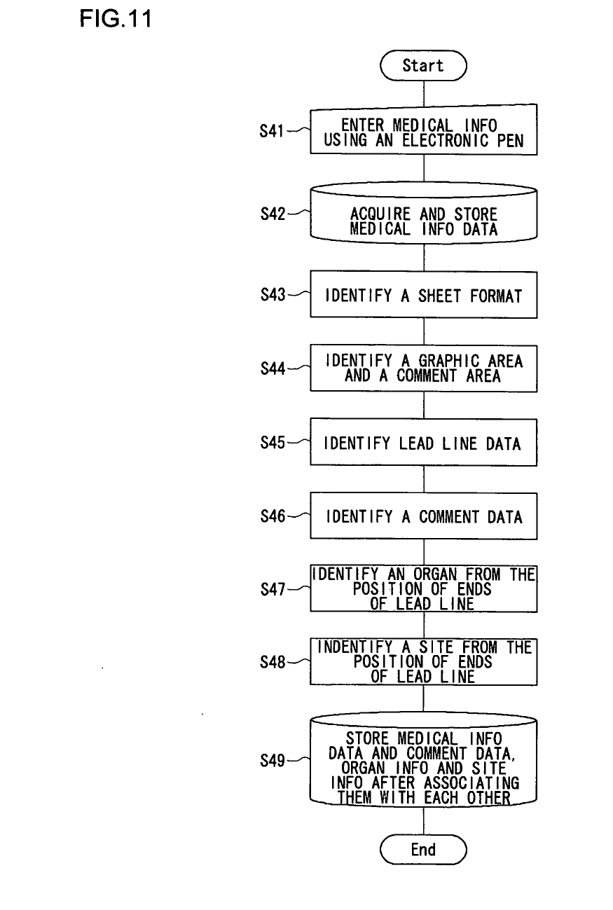

FIG. 11 is a flowchart showing processings performed on medical information data by a medical information management system 200 according to a sixth embodiment of the present invention. The description of S41 to S44, which is the same as that of S31 to S34 in FIG. 8, will be omitted.

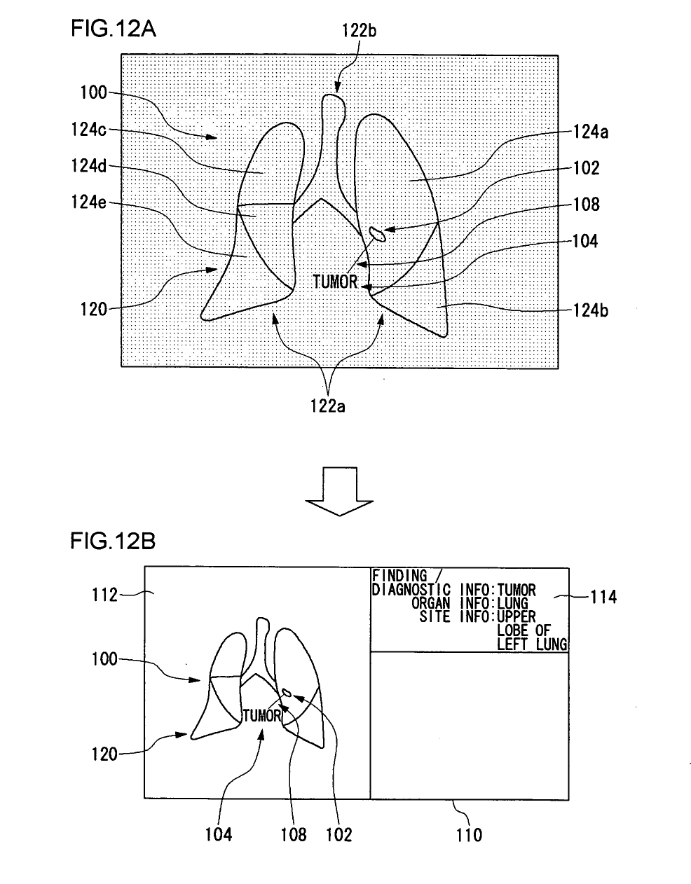

Upon identification of the graphic area and the comment area, the lead line data identification unit 68 identifies lead line data from among the medical information data (S45). The lead line data identification unit 68 identifies, as lead line data, medical information data which represents a line having a length equal to or longer than a predetermined length and with one end thereof located in a graphic area and the other end thereof in a comment area. In the example of FIG. 12A, the lead line 108 is such that the one end thereof is located inside a first organ area 122a identified as a graphic area and the other end thereof is located in the comment area, namely outside the first organ area 122a and second organ area 122b. Accordingly, the lead line data identification unit 68 identifies the lead line 108 as lead line data.

Upon identification of lead line data, the comment data identification unit 70 identifies comment data (S46). Similar to the above described, the comment data identification unit 70 identifies, as comment data, medical information data representing medical information which is located in the comment area and at the same time closer than or equal to a predetermined distance to one end of the lead line represented by the identified lead line data. In the example of FIG. 12A, a comment 104 is entered in a comment area, namely outside the first organ area 122a and second organ area 122b. Also, the comment 104 is located closer than or equal to a predetermined distance to one end of the lead line 108. Accordingly, the comment data identification unit 70 identifies the medical information data representing the comment 104 as the comment data. The text conversion unit 72 carries out a text conversion on the thus identified comment data.

Upon identification of a comment area, the organ identification unit 76 identifies an organ on which a comment is to be made, from the position of ends of a lead line (S47). In the sixth embodiment, the organ identification unit 76 compares an organ area contained in the organ area information 56 with the position of ends of a lead line represented by the identified lead line data. Thereby, which organ area the lead line represented by the identified lead line data is located at is determined so as to identify the organ on which a comment is to be made. In the example of FIG. 12A, an end of the lead line 108 is located inside the first organ area 122a indicating a lung organ. Accordingly, the organ identification unit 76 identifies that the organ, on which to be commented, located near the other end of the lead line 108 is a “lung”.

When an organ is specified from the location of the ends of a lead line, the site identification unit 78 identifies a site from the position of the ends of the lead line (S48). According to the sixth embodiment, the organ identification unit 76 compares the area of an organ contained in the site area information 58 with the position of a lead line represented by the identified lead line data. As a result thereof, which site area the lead line represented by the identified lead line data is located at is determined so as to identify a site on which a comment is to be made. In the example of FIG. 12A, an end of the lead line 108 is located inside a first site area 124a indicating an upper lobe of left lung. Thus, site identification unit 78 identifies that the organ, located near the other end of the lead line 108, on which a comment shall be made is “upper lobe of left lung”.

Upon identification of an organ and a site on which a comment shall be made, the storage unit 44 stores the medical information data and the comment data, organ information and site information after associating them with each other (S49). Thus, as shown in FIG. 12B, the medical information 100 can be displayed on a patient's chart screen 110, and the comment data converted into text information, the organ information and the site information can be displayed on a comment information screen 114, when these pieces of information are to be browsed from a server 40 or from a workstation 30 belonging to any of the divisions. In the example of FIG. 12B, comment data in characters “tumor”, organ information in characters “lungs”, and site information in characters “upper lobe of left lung” are displayed as text information.

As described above, a comment, such as a physician's opinion or the results of diagnosis, is first displayed on the comment information screen 114 in distinction from the schema, so that it is possible to check the comment, such as a physician's opinion or the results of diagnosis, simply by looking at the comment information screen 114. Thereby, the comment can be checked quickly, thus providing an efficient medical environment to physicians and the like. Also, the comment information, which comes converted to text information, provides an easy-to-understand display of comment to those who see the comment information screen 114. Since the comment information has been converted into the text information, the enlargement of characters of comment information and the change of font or color and so forth can be done easily, thus further improving the easy-to-understand display of comment. Also, since organ information and site information related to the comment are displayed, the comment can be viewed, in the state where the comment is associated with an organ or site, by simply looking at the comment information screen 114. As a result, the details of the medical information 100 can be grasped by looking at the comment information screen 114. In this manner, identifying an organ or site from the medical information inputted in handwriting enables the effective use of the medical information data representing the manually inputted medical information. In the example of FIG. 12B, a person who browses the medical information screen 112 can grasp the result of diagnosis that there is a tumor in the upper lobe of left lung, by looking at the comment information screen 114. In this manner, it is possible to effectively use the medical information data representing the medical information manually inputted in the case when the form already printed with a figure of an organ is used and furthermore the comment is entered using lead lines.

### Seventh Embodiment

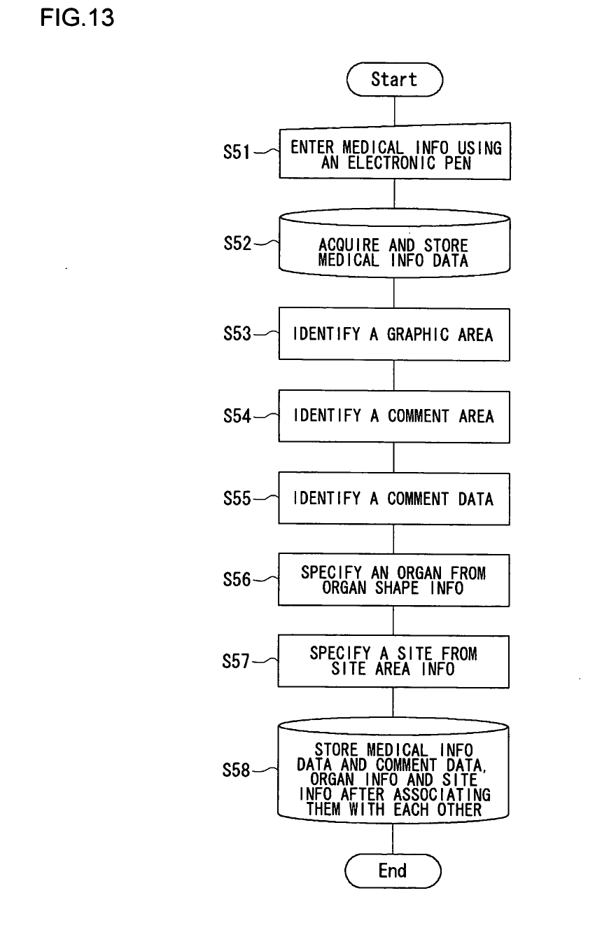

FIG. 13 is a flowchart showing processings executed on medical information data by a medical information management system 200 according to a seventh embodiment of the present invention. The description of S51 to S55, which is the same as that of S11 to S15 in FIG. 2, will be omitted.

When a schema is inputted in handwriting onto a form 10 by an electronic pen 12, a graphic data identification unit 66 identifies the manually inputted schema as graphic data. An organ identification unit 76 identifies an organ represented by the manually inputted schema, from the graphic data identified by the identified graphic data and the organ form information 48 stored in the storage unit 44 (S56). In the seventh embodiment, the organ identification unit 76 compares the graphic form represented by the identified graphic data with the shapes of an organ represented by various organ form data contained in the organ form information 48. Of the shapes of an organ represented by the organ form data contained in the organ form information 48, one which is closest to the shape represented by the identified graphic data is identified as the organ represented by the graphic data.

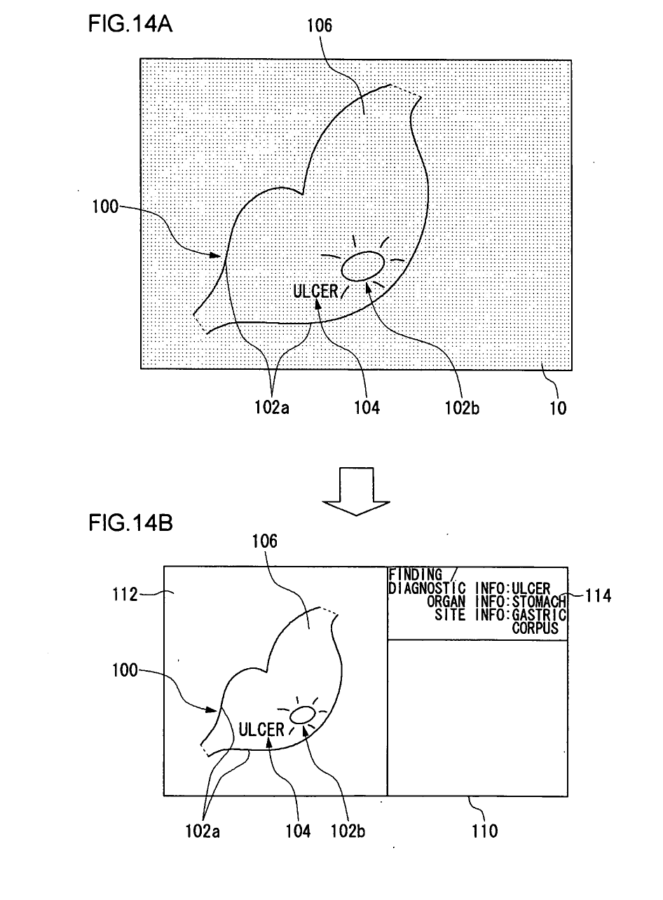

In the example shown in FIG. 14A, an organ graphic 102a representing the shape of a stomach is inputted manually onto the form 10. Firstly, the graphic data identification unit 66 identifies this organ graphic 102a as graphic data, from the line length or the like. The organ identification unit 76 compares the shape of this organ graphic 102a with the shapes of an organ represented by the organ form data contained in the organ form information 48. The organ form information 48 contains the organ form data that indicates the shape of a stomach, and the organ identification unit 76 identifies an organ, indicated by the organ graphic 102a, as a stomach.

From the identified graphic data and the site area information stored in the storage unit 44 the site identification unit 78 identifies a site, on which a comment is to be made, in the manually inputted schema (S57). In the seventh embodiment, the site identification unit 78 refers to the site area information 50 about an area of the site of the organ identified by the organ identification unit 76. From the position of identified comment data and the area of a site represented by the site area data contained in the site area information 50, the site identification unit 78 identifies which area the identified comment data are entered into. As a result, when the schema and the comment are inputted in handwriting, the site on which a comment is to be made is identified.

In the example shown in FIG. 14A, the organ is identified, as a stomach, by the organ identification unit 76 and thus the site identification unit 78 refers to the site area information on a stomach. From the position, at which the comment 104 composed of the character “ulcer” was inputted in handwriting, and the site area information on a stomach, the site identification unit 78 identifies a location where the comment 104 was written to any of site areas among the cardiac part of stomach, gastric corpus, pyloric part of stomach and so forth. In the example of FIG. 14A, the comment 104 is located at the gastric corpus and therefore it is identified that the comment 104 is written onto the gastric corpus.

As the organ and the site are identified, the medical information data are associated with the comment data, the organ information indicative of the identified organ and the site information indicative of the identified site, and then stored in the storage unit 44 (S58). When these pieces of information are to be browsed by the server 40 or a workstation 30 belonging to any of the divisions, the medical information 100 is displayed on the patient's chart screen 110, and the comment data converted into the text, the organ information and the site information are displayed on the comment information screen 114. In the example of FIG. 14B, “ulcer”, “stomach” and “gastric corpus” are displayed on the comment information screen 114 as the comment data, organ information and the site information, respectively.

As described above, a comment, such as a physician's opinion or the results of diagnosis, is first displayed on the comment information screen 114 in distinction from the schema, so that it is possible to check the comment, such as a physician's opinion or the results of diagnosis, simply by looking at the comment information screen 114. Thereby, the comment can be checked quickly, thus providing an efficient medical environment to physicians and the like. Also, the comment information, which comes converted to text information, provides an easy-to-understand display of comment to those who see the comment information screen 114. Since the comment information has been converted into the text information, the enlargement of characters of comment information and the change of font or color and so forth can be done easily, thus further improving the easy-to-understand display of comment. Also, since organ information and site information related to the comment are displayed, the comment can be viewed, in the state where the comment is associated with an organ or site, by simply looking at the comment information screen 114. As a result, the details of the medical information 100 can be grasped by looking at the comment information screen 114. In the example of FIG. 14B, a person who browses the medical information screen 112 can grasp the result of diagnosis that there is an ulcer in the gastric corpus, by simply looking at the comment information screen 114. In this manner, identifying an organ or site from the medical information inputted in handwriting enables the effective use of the medical information data representing the manually inputted medical information.

The present invention is not limited to each of the above-described embodiments and the combination of each constituent element in each embodiment may be combined as appropriate and the combination thereof may serve effective as an embodiment of the present invention. It is also possible to add modification with a variety of changes in designing and so forth to each embodiment, based on the knowledge of the skilled in the art. The embodiments added with such modifications are also within the scope of the present invention. Such modification will be described hereinbelow.

The medical information management system 200 may acquire the medical information data representing the manually inputted medical information by use of a scanner. In such a case, the form will function as an input medium and the scanner will function as a means for acquiring medical information data. Also, the medical information management system 200 may acquire the medical information data representing the manually inputted medical information by use of a graphic tablet, a digitizer, or a tablet PC capable of inputting to the screen. In such a case, each of these devices will function as an input medium and a means for acquiring medical information data, and the input surface and screen of these devices will function as input area. As a result, it is possible to easily acquire the manually inputted medical information as medical information data.

Now, the background technology for the eighth embodiment to the twenty-first embodiment will be described. In the field of medicine, it has been a general practice to record medical information, such as findings and diagnostic information, in a patient's chart made of paper. In recent years, however, electronization or computerization of patients' charts by use of personal computers has been finding quickly widening use helped by the technological innovation in the medical field. Accordingly, the technology in which to utilize the electronic information of the electronic patient's chart and achieve the provision of the efficient and accurate medical care is in demand. In this regard, there has been proposed, for example, a medical information inputting system in Reference (1) in the following Related Art List where an electronic pen is used to make an entry onto a particular form so as to acquire the contents entered onto the form and the server performs the processing for verifying a patient against the acquired data and the like. For example, in Reference (2) a patient's chart management system is proposed which acquires the data representing the contents entered onto the special form by using an electronic pen and which registers additional data if there is any additional data entered onto the form.

Related Art List

(1) Japanese Patent Application Laid-Open No. 2004-30257.

(2) Japanese Patent Application Laid-Open No. 2004-54375. References (1) and (2) disclose a technology for acquiring data representing the records entered in a specific form with an electronic pen and entering the acquired data in a database. However, technology for further utilizing such data is not disclosed. Therefore, it is strongly desired that technology be developed for further utilizing electronic information representing medical information.

The eighth to the twenty-first embodiment of the present invention have been made to solve the problems as described above, and a general purpose thereof is to provide a technology for effectively utilizing electronic information representing medical information which has been inputted in handwriting.

In order to solve the above problems, a medical information management system according to one embodiment of the eighth to the twenty-first embodiment of the present invention comprises: a medical information data acquiring means which acquires medical information data inputted in handwriting; an attribute extracting means which extracts, from the acquired medical information data, attribute information that identifies a state in which the medical information data are inputted in handwriting; a processing decision means which determines a processing to be performed on the medical information data, using the extracted attribute information; and a processing execution means which performs the determined processing. According to this embodiment, the medical data which have been inputted in handwriting can be utilized effectively. The processing decision means may determine a processing performed on the medical information data having the extracted attribute information. According to this embodiment, the medical information data on which the processing is to be performed can be identified in a simplified manner, so that the processing can be performed smoothly on the medical information data. The processing decision means may determine a processing performed on the medical information, having no extracted attribute information, which correspond to the medical information data having the extracted attribute information.

It is to be noted that any arbitrary combination of the above-described structural components and expressions of the eighth to the twenty-first embodiment replaced mutually between a computer program, a recording medium storing the computer program and so forth are also effective as the eighth to the twenty-first embodiment.

Hereinbelow, a detailed description will be given of eighth to twenty-first embodiments with reference to drawings.

A medical information processing system according to the eighth to the twenty first embodiment will be given before a specific description of the eighth to the twenty-first embodiment. Firstly, a user manually inputs medical information onto a form 1010, using an electronic pen 1012. In the medical information processing system according to the eighth to the twenty first embodiment, the medical information entered onto the form 1010 is acquired as medical information data, and the attribute information for identifying the condition of manual input of the medical information data is extracted from the acquired medical information data. Further, the processing to be done on the medical information data is determined based on the extracted attribute information.

### Eighth Embodiment

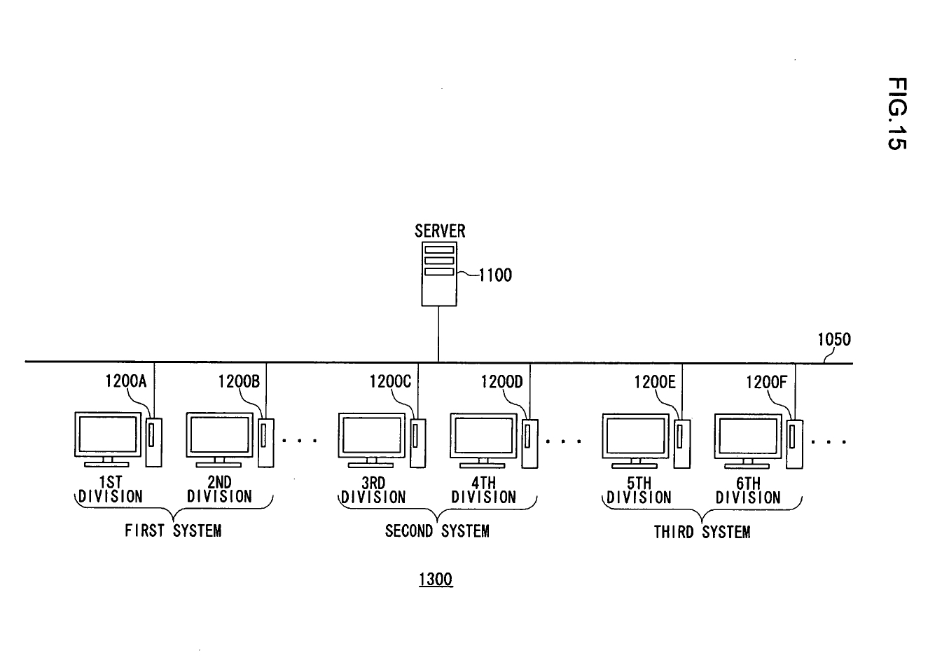

FIG. 15 illustrates a general structure of an electronic patient's chart system 1300 according to an eighth embodiment. In the field of medicine, a medical system may have a plurality of systems, and each of the plurality of systems may have a plurality of divisions. A medical system may, for instance, have a plurality of systems, such as an inspection system, a ward system and a support system. Further, the inspection system may have a plurality of divisions, such as an endoscopic inspection division and a pathology laboratory division. Also, the ward system may have a plurality of hospital wards, such as an east ward and a north ward, whereas the support system may have a plurality of divisions, such as a medical accounting division and a pharmaceutical division.

An electronic patient's chart system 1300 as a medical information processing system according to the eighth embodiment has a workstation 1200 in each of the respective divisions of a medical system like this. In the example of FIG. 15, a first system has a first division and a second division, and a first workstation 1200A is installed in the first division and a second workstation 1200B is installed in the second division. Similarly, a second system has a third division and a fourth division, and a third workstation 1200C is installed in the third division and a fourth workstation 1200D is installed in the fourth division. Likewise, a third system has a fifth division and a sixth division, and a fifth workstation 1200E is installed in the fifth division and a sixth workstation 1200F is installed in the sixth division.

The workstations 1200A to 1200F and so forth (hereinafter generically referred to as “workstation 1200” also) are connected with one another via a network 1050. With this structure, each of the workstations 1200 can transmit and receive data to and from one another. The workstations 1200 are each connected to a server 1100 via the network 1050. The server 1100 contains electronic patients' charts. Accordingly, the workstations 1200 can each access the data of the server 1100 to view the electronic patients' charts. Furthermore, the workstations 1200 each transmit and receive data to and from the server 1100, so that various processing can be performed using electronic information available in the electronic patients' charts.

Let us suppose that the first system is an inspection system, the second system a ward system, and the third system a support system. Let us also suppose that the first division is an endoscopic inspection division; the second division is a pathology laboratory division; the third division is an east ward; the fourth division is a north ward; the fifth division is a medical accounting division; and the sixth division is a pharmaceutical division. In this case, users in each of these divisions can access electronic patients' charts in the server 1100 via the network 1050. For example, the medical accounting division can obtain information on inspections performed by the endoscopic inspection division by accessing relevant electronic patients' charts and use them in medical accounting. Also, for example, the pharmaceutical division can obtain information on the results of diagnoses at each of the hospital wards by accessing relevant electronic patients' charts and use them in its pharmacological practice.

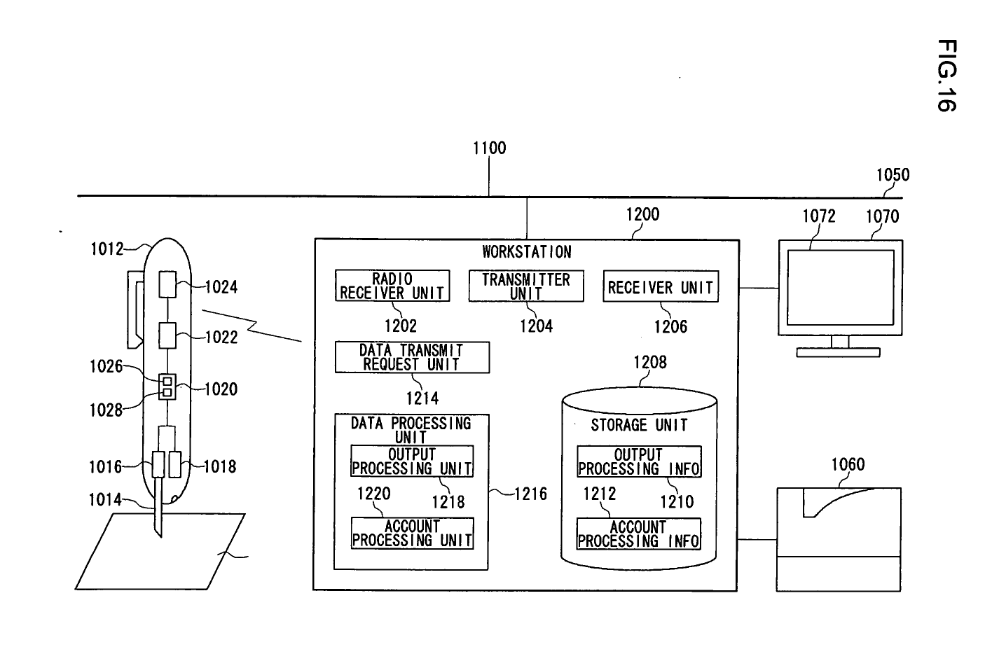

FIG. 16 illustrates a general structure of an electronic pen 1012 and a workstation 1200 according to an eighth embodiment of the present invention. In addition to a structure common to that shown in FIG. 15, FIG. 16 further includes account processing information 1212 and an account processing unit 1220 which are specific for a workstation 1200 in the accounting division in particular. As a user makes a handwritten input onto a form 1010 in an electronic patient's chart system 1300 using an electronic pen 1012, the electronic pen 1012 can acquire the content of entry onto the form 1010 successively. Thus, the form 1010 functions as an input medium through which medical information data are manually inputted, and the electronic pen 1012 functions as a data acquisition means for acquiring the content of information inputted onto the form 1010 as data. This electronic pen 1012 includes a pen part 1014, a pressure-sensing part 1016, a camera part 1018, a processing part 1020, a storage part 1022, and a radio transmission part 1024.

The pen part 1014 carries out a recording onto the form 1010 by emitting ink as the pen point pressed to the form 1010 is moved. The pressure-sensing part 1016, which is fixed to the pen part 1014, has a piezoelectric element and thereby detects forces and pressures that work on the pen part 1014. When an entry onto the form 1010 is being made by the pen part 1014, the pressure-sensing part 1016 detects the handwritten input being made by the electronic pen 12 because the pen part 1014 is pressed against the form 1010. The camera part 1018, using infrared rays, reads the dot pattern printed on the paper surface of the form 10 around the tip of the pen part 1014.

The form 1010 functions as an input means with which to enter a handwritten input by this electronic pen 1012. The form 1010 has a special dot pattern, printed with an ink containing carbon, formed thereon. The area or field where this dot pattern is formed is an input area where a user inputs medical information using the electronic pen 1012. Medical information meant here is information entered onto the form by a physician or the like, which includes, for instance, patient information such as a patient's name attending physician's name, date of diagnosis, the physician's findings, diagnostic results, tick marks and strikethroughs entered onto a sheet of patient's chart.

Each of the dots of a dot pattern printed in the input area is arranged two-dimensionally in such a manner that its location can be identified. As a manual input onto this form 1010 is initiated by the electronic pen 1012, the pressure-sensing part 1016 first detects the manual input being made by the electronic pen 1012. While the manual input is being detected by the pressure-sensing part 1016, the camera part 1018 reads the dot pattern on the form 1010. In this manner, the electronic pen 1012 can acquire the locus of the tip of the pen part 1014 during the manual input and thereby acquire the content of handwritten entry onto the form 1010 as data. Accordingly, when medical information, such as a physician's opinion or diagnostic results, is hand-written onto the form 1010 with an electronic pen 1012, the electronic pen 1012 can acquire the manually inputted medical information as medical information data representing this medical information.

Each form 1010 has its own form ID. The electronic pen 1012 can acquire this form ID by reading the dot pattern. Hence, even when some additional entry is made onto the same form 1010, data representing the content of previous entry can be easily identified and the additional data can be easily associated with the previous data. As will be discussed later, this form ID can also be used to identify the form. Note that each form 1010 may not have its own form ID, and the user may set the form ID. In such a case, the user may make a handwritten input onto a predetermined form ID entry space of the form 1010, for example, so that the user may set the form ID. Also, the user may set the form ID in the medical data stored in the server 1100 by use of an input device such as a keyboard or mouse.

The processing part 1020 performs processings such as data compression and addition of the ID of the electronic pen 1012 for the data read by the camera part 1018. This processing unit 1012 has a pen ID assignment unit 1026. The electronic pen 1012 has its own ID and this ID is stored in a pen ID storage (not shown). The processing part 1020 acquires a pen ID stored in this pen ID storage and appends the acquired pen ID to the medical information data read out by the camera part 1018. Accordingly, the server 1100 can determine which electronic pen 1012 was used to input the medical data.

The processing part 1020 has a time information assignment part 1028. The processing part 1020 has a clock part which measures the time. The processing part 1020 measures dates and time inputted by the electronic pen 1012, by this clock part and appends entry time information indicative of the date and time of measurement to the medical information data. Accordingly, the date and time at which the medical information data was inputted by the electronic pen 1012 can be determined using the entry time information appended to the medical information data. It is to be noted that those in which additional information such as a pen ID or entry time information is appended to the medical information data also constitute the medical information data.

The storage part 1022 stores data on which a processing has been performed by the processing part 1020. The radio transmission part 1024 transmits information stored in the storage part 1022 to the outside wirelessly by Bluetooth (registered trademark). The electronic pen 1012 has a USB (Universal Serial Bus) interface (not shown). Accordingly, the electronic pen 1012 can also output data through wire to the outside using a USB cable.

The workstation 1200 includes a radio receiver unit 1202, a transmitter unit 1204, a storage unit 1208, a data transmit request unit 1214, a data processing unit 1216 and so forth. The radio receiver unit 1202 receives data transmitted wirelessly from the electronic pen 1012. The storage unit 1208 stores the data received by the radio receiver unit 1202. The transmitter unit 1204 transmits the medical data received by the radio receiver unit 1202 to the server 1100 or workstations 1200 of other divisions via the network 1050. Thus, the workstation 1200 has the function of a data relay means by receiving data from the electronic pen 1012 and outputting data to the server 1100. The receiver unit 1206 receives data from the server 1100 or workstations 1200 of other divisions via the network 1050.

The storage unit 1208 has output processing information 1210 and account processing information 1212. The output processing information 1210 includes predefined information that enables the output of medical information data stored in the storage unit 1208 or medical information data received from the server 1100 or the like to a printer 1060 or a display 1070. The account processing information 1212 includes predefined information that enables account processing based on medical information data received from the server 1100 or the like. This account processing information 1212 is utilized mainly by the medical accounting division.

The data transmit request unit 1214 requests transmission of medical information data or data related thereto from the server 1100 or the like. The workstation 1200 is provided with a data transmit request input means (not shown) for accepting input from the user to request transmission of data from the server 1100 or from a workstation 1200 belonging to another division. This data transmit request input means is, for instance, displayed as a transmission request button on the screen 1072 of the display 1070. When the transmission request button is pressed by the user through a keyboard or a mouse, the data transmit request unit 1214 requests transmission of data from the server 1100 or a workstation 1200 of another division whichever has the data required. In this manner, the users of a workstation 1200 can request transmission of data when they want to obtain the data, and thus this system accomplishes transmission and reception of data reflecting the will or desire of its users.

The data processing unit 1216 carries out processing on data received from the server 1100 or a workstation 1200 belonging to another division, data received from the electronic pen 1012, or data stored in the storage unit 1208. The data processing unit 1216 includes an output processing unit 1218. The output processing unit 1218 carries out an output processing for outputting medical information data stored in the storage unit 1208 or medical information data received from the server 1100 or the like to the printer 1060 or the display 1070. The data processing unit 1216 also includes an account processing unit 1220. The account processing unit 1220 performs accounting processing based on medical information data received from the server 1100 or the like. Accordingly, the account processing unit 1220 functions as a data processing means for processing medical information data. This account processing unit 1220 is utilized in the medial accounting division.

The printer 1060 is connected to the workstation 1200. The printer 1060 makes a printed output of medical information data received from the server 1100 or a workstation 1200 belonging to another division via the network 1050, medical information data received from the electronic pen 1012, or medical information data stored in the storage unit 1208. The display 1070 is also connected to the workstation 1200. The display 1070 outputs a display of these medical information data on the screen 1072.

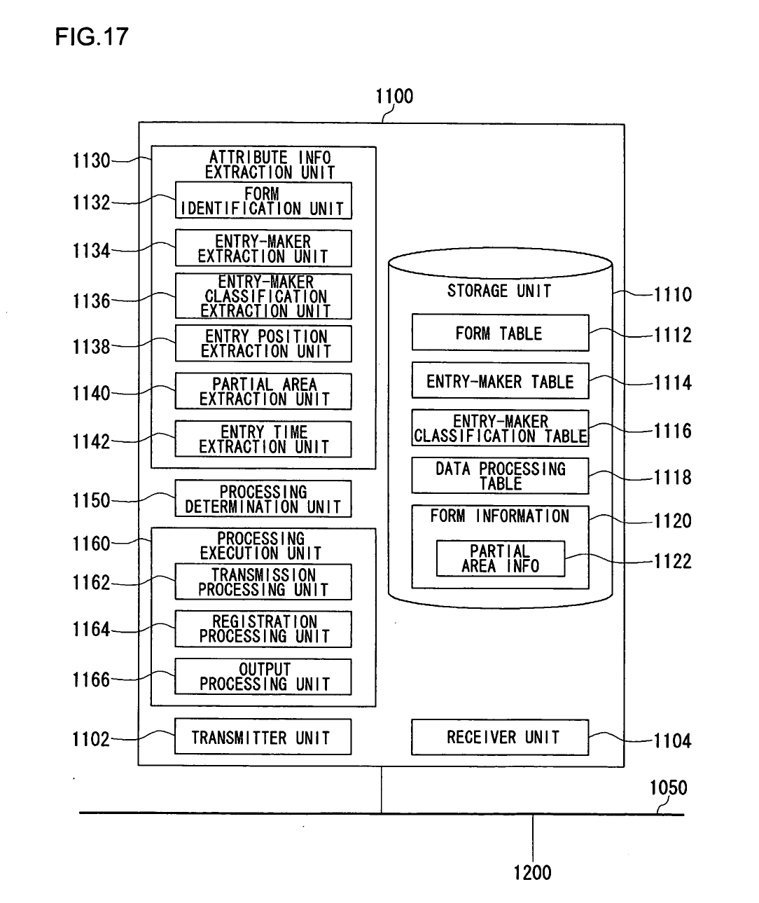

FIG. 17 illustrates a general structure of the server 1100 according to the eighth embodiment of the present invention. The server 1100 includes a transmitter unit 1102, a receiver unit 1104, a storage unit 1110, an attribute information extraction unit 1130, a processing determination unit 1150, a processing execution unit 1160 and so forth.

The server 1100, which is connected to the network 1050, is coupled to workstations 1200 installed in the respective divisions via the network 1050. The transmitter unit 1102 transmits medical information data and other data to workstations 1200 of the respective divisions via the network 1050. Accordingly, the transmitter unit 1102 functions as a transmission means for transmitting medical information data to such data processing means as the account processing unit 1220. The receiver unit 1104 receives medical information data and other data from workstations 1200 of the respective divisions via the network 1050.

The storage unit 1110 stores medical information data received from the receiver unit 1104. The storage unit 1110 stores beforehand a form table 1112, an entry-maker table 1114, an entry-maker classification table 1116, a data processing table 1118, form information 1120 and so forth.

The form table 1112 includes a table of form IDs associated with electronic patients' charts. Hence, by extracting a form ID from acquired medical information data, it becomes possible to identify form information of manually inputted medical information data from among form information stored in the storage unit 1110. Accordingly, when, for instance, medical information is added onto a form 1010 with an electronic pen 1012, the server 1100 can identify the form with the manually inputted medical information data and the form information associated with the form, and thus can decide whether to store the additionally inputted medical information data in association with the form information or not.

The entry-maker table 1114 includes a table of entry-maker information representing entry-makers associated with pen IDs. Hence, by using the pen ID added to medical information data and the entry-maker table 1114, it becomes possible to extract entry-maker information from the acquired medical information data and identify the person who has made the entry of the medical information data. In an electronic patient's chart system 1300 according to the eighth embodiment, the individual users are provided with their own electronic pens 1012 for the effective use thereof. Each user makes a handwritten input onto a form 1010, using an electronic pen 1012 assigned to him/her. In this eighth embodiment, therefore, the entry-maker information associated with pen IDs represents the owners of the respective electronic pens 1012. Accordingly, if an entry-maker is to be identified accurately using a pen ID, any change in the ownership of the electronic pen 1012 must be quickly reflected in the entry-maker information.

The entry-maker classification table 1116 includes a table of entry-maker classification information associated with pen IDs. Hence, by using the pen ID added to medical information data and the entry-maker classification table 1116, it becomes possible to extract entry-maker classification information from the acquired medical information data and identify the classification of the person who has made the entry of the medical information data. The entry-maker classification information, which is the information for classifying the entry-makers, includes, for example, information representing physicians, information representing nurses, information representing medical accounting staff, and information representing pharmaceutical staff. Further, the information representing physicians may include information representing advising doctors, information representing trainee doctors, information representing laboratory doctors, and the like. The entry-maker classification information associated with pen IDs can also be changed by the users anytime by rewriting the entry-maker classification table 1116.

The data processing table 1118 includes a table in which attribute information is associated with processing to be performed based on medical information data. The attribute information, which is the information for identifying the condition of manual input of medical information data, concerns, for instance, the entry-maker who has made the manual input of the medical information data, the classification of the entry-maker who has made the manual input of the medical information data, the position, or partial area, of the manual input of the medical information data, and the entry timing, or date and time, of the manual input of the medical information data. Thus the attribute information is set for a plurality of items, and the attributes under such items constitute the attribute information of medical information data. The data processing table 1118 also includes a table in which a plurality of predefined attribute information are associated with a processing to be performed based on medical information data. Accordingly, it is possible to determine the processing to be performed on medical information data, based on the attribute information extracted from the acquired medical information data.

The form information 1120 includes partial area information 1122. The partial area information 1122 includes partial area information representing partial areas of the respective forms of patients' charts. Partial areas are areas representing the parts or whole of the input area of a form 1010 and include various entry spaces, for instance. Thus, by extracting the position of entry on the form 1010 from the acquired medical information data, it is possible to identify which of the partial areas of the form 1010 has received the entry of the medical information by the user.

The attribute information extraction unit 1130 extracts attribute information for identifying the condition of manual input of medical information data by a user using an electronic pen 1012 and a form 1010 from the medical information data acquired from the electronic pen 1012. The condition and the state of manual input of medical information data meant here includes, for instance, who has made the manual input of the medical information data, when he/she has done it, or what authority the person has for the manual input of the medical information data, and the like. The attribute information extraction unit 1130 includes a form identification unit 1132, an entry-maker extraction unit 1134, an entry-maker classification extraction unit 1136, an entry position extraction unit 1138, a partial area extraction unit 1140, and an entry time extraction unit 1142.

The form identification unit 1132 extracts a form ID from the acquired medical information data. The form identification unit 1132 identifies form information corresponding to the acquired medical information data by using the extracted form ID and the form table 1112. The entry-maker extraction unit 1134 extracts a pen ID from the acquired medical information data. The entry-maker extraction unit 1134 extracts entry-maker information representing the person who has manually inputted the acquired medical information data by using the extracted pen ID and the entry-maker table 1114. The entry-maker classification extraction unit 1136 extracts a pen ID from the acquired medical information data. The entry-maker classification extraction unit 1136 extracts entry-maker classification information representing the classification of the person who has manually inputted the acquired medical information data by using the extracted pen ID and the entry-maker classification table 1116.

The entry position extraction unit 1138 extracts entry position information representing the position of extraction of the acquired medical information data from the acquired medical information data. The form identification unit 1132 identifies a form from the form information in the manually inputted medical information data. The partial area extraction unit 1140 extracts partial area information representing the partial area where the acquired medical information data have been entered by using the entry position information extracted by the entry position extraction unit 1138 and the partial area information 1122 in the identified form, which is stored in the storage unit 1110. The entry time extraction unit 1142 extracts, from the acquired medical information data, entry time information representing the date and time of manual input of the medical information data by the user('s) using an electronic pen 1012 and a form 1010.

The processing determination unit 1150 determines the processing to be performed on the medical information data by using the attribute information extracted by the attribute information extraction unit 1130 and the data processing table 1118. The processing execution unit 1160 executes processing determined by the processing determination unit 1150 on the medical information data. The processing execution unit 1160 includes a transmission processing unit 1162, a registration processing unit 1164, and an output processing unit 1166.

The transmission processing unit 1162 executes transmission processing determined by the processing determination unit 1150. The transmission processing unit 1162 not only transmits predefined medical information data to a workstation 1200 or the like, but also communicates predefined matters to a workstation 1200 or the like.

The registration processing unit 1164 executes registration processing determined by the processing determination unit 1150. Here, registration processing means the creation of a state where medical information data can be classified into registered ones and nonregistered ones or can be classified by the type of registration. More specifically, registration processing includes, for instance, adding a temporary registration identification data representing a temporary registration or a formal registration identification data representing a formal registration to medical information data for storage in the storage unit 1110. It also includes, for instance, adding requested information identification data representing requested information or reported information identification data representing reported information to medical information data.

The output processing unit 1166 determines output format or output conditions by using, for instance, attribute information extracted by the attribute information extraction unit 1130 from the acquired medical information data. The output format includes, for instance, printed output forms and displayed output forms. The output conditions include, for instance, the conditions for printed output or displayed output of added or deleted medical information data. Accordingly, the output processing unit 1166 makes a printed output of medical information data stored in the storage unit 1110 to a network printer (not shown) or the like connected to the network 1050 or makes a displayed output thereof to a display (not shown) connected to the server 1100.

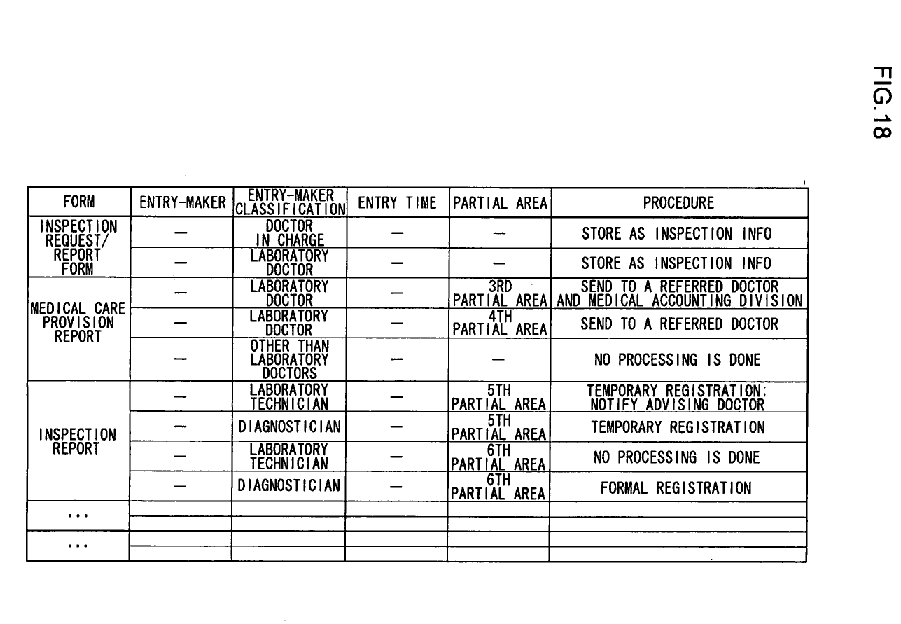

FIG. 18 illustrates a data processing table 1118 stored in a storage unit 1110 in an electronic patient's chart system 1300 according to the eighth embodiment. A form identification unit 1132 identifies the form printed on a sheet of paper in which the medical information data have been inputted manually, by identifying the form information stored in the storage unit 1110 by using a form ID. The data processing table 1118 also specifies processings that are associated with a plurality of attribute information, which includes entry-makers who make entries in this form, entry-maker classifications, entry times, and processings associated with a plurality of attribute information containing the partial areas entered.

As described above, the processings to be executed on medical information data are associated with attribute information as constituent factors for the decision on processing. The processing determination unit 1150 refers to this data processing table 1118 when determining the processing to be executed on certain medical information data. The processing determination unit 1150 determines the processing to be executed on certain medical information data according to a combination of a plurality of attribute information as constituent factors contained in the data processing table 1118. In this case, the processing determination unit 1150 may determine the processing to be executed on certain medical information data according to the combination of all the attribute information as constituent factors contained in the data processing table 1118. Also, the processing determination unit 1150 may determine the processing to be executed on certain medical information data according to a combination of two or more of the constituent factors of attribute information out of all the attribute information as constituent factors contained in the data processing table 1118.

For example, the processing determination unit 1150 determines the processing to be executed on the medical information data according to a combination of two constituent factors, namely, entry-maker classification and partial area, out of all the attribute information as constituent factors contained in the data processing table 1118. If, as shown in FIG. 18, the identified form is an inspection request/report form and the entry-maker classification information extracted from acquired medical information data is a physician in charge, then the processing determination unit 1150 will refer to the data processing table 1118 and decide on the processing of storing the acquired medical information data as inspection request information. Also, if the identified form is an inspection request/report form and the entry-maker classification information extracted from acquired medical information data is a laboratory doctor, then the processing determination unit 1150 will refer to the data processing table 1118 and decide on the processing of storing the acquired medical information data as an inspection report information in the storage unit 1110.

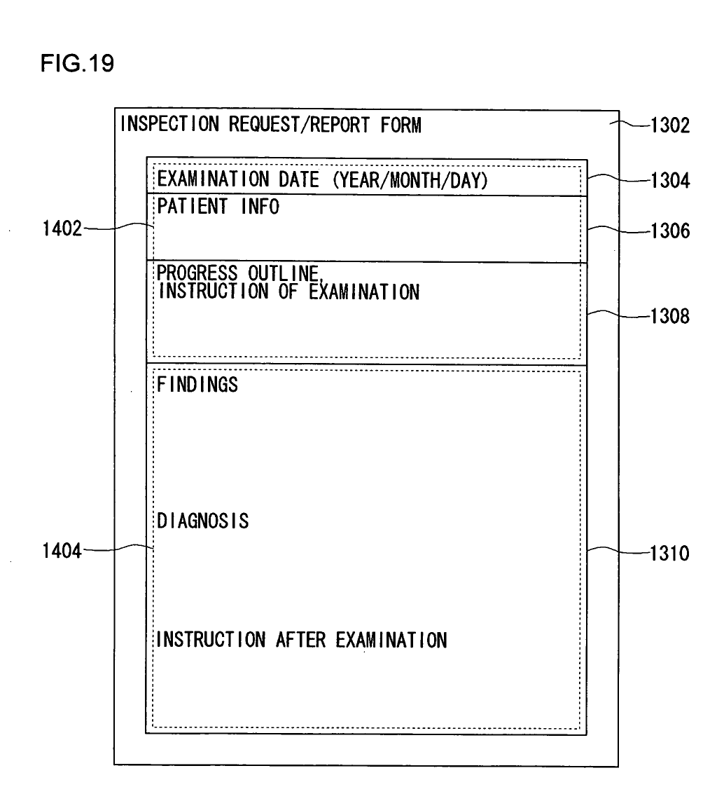

FIG. 19 illustrates an inspection request/report form 1302 used in the electronic patient's chart system 1300 according to the eighth embodiment. The inspection request/report form 1302 has an examination date space 1304, a patient information space 1306, a progress outline space 1308 and finding/diagnosis space 1310.

In the inspection request/report form 1302 like this, the physician in charge normally makes an entry of an examination request in the examination data space 1304, the patient information space 1306 and the progress outline space 1308, and then requests a laboratory doctor to carry out an inspection. Of the inspection request/report form 1302, partial areas in which the doctor in charge makes an entry of an inspection request is called here a first partial area 1402. The inspection request/report form 1302 in which those spaces are filled in by the doctor in charge is delivered to a laboratory doctor. Based on the inspections requested in the thus delivered inspection request/report form 1302, the laboratory doctor carries out an examination and fills in the finding/diagnosis space 1310 with an inspection report. Of the inspection request/report form 1302, partial areas in which the laboratory doctor makes an entry of an inspection report is called here a second partial area 1404. The inspection request/report form 1302 in which the inspection report has been entered in the finding/diagnosis space 1310 is delivered back to the doctor in charge, so that the result of an examination is reported to him/her.

If an inspection request entered by a doctor in charge in an inspection request/report form 1302 like this is registered as inspection request information in the electronic patient's chart system 1300, then a laboratory doctor can browse the inspection request information at the workstation 1200 of his/her division even before the inspection request/report form 1302 is delivered to him/her. Also, if an inspection report entered by a laboratory doctor is registered as inspection report information in the electronic patient's chart system 1300, then the doctor in charge can browse the inspection report information at the workstation 1200 of his/her division even before the inspection request/report form 1302 is delivered to him/her. Note here that a physician in charge and a doctor in charge are used interchangeable in this patent specification.

Accordingly, an inspection request/report form 1302 used in the electronic patient's chart system 1300 according to the present embodiment is prepared in such a manner that the form of an inspection request/report form 1302 is printed on a form 1010 already printed with a dot pattern which is readable by an electronic pen 1012. In the electronic patient's chart system 1300 according to the present embodiment, when medical information data are manually inputted in an inspection request/report form 1302 by a physician in charge, the medical information data are registered as inspection request information. Also, when medical information data are manually inputted in an inspection request/report form 1302 by a laboratory doctor, the medical information data are registered as inspection report information. As a result, it is possible to effectively use the medical information data inputted in the inspection request/report form 1302.

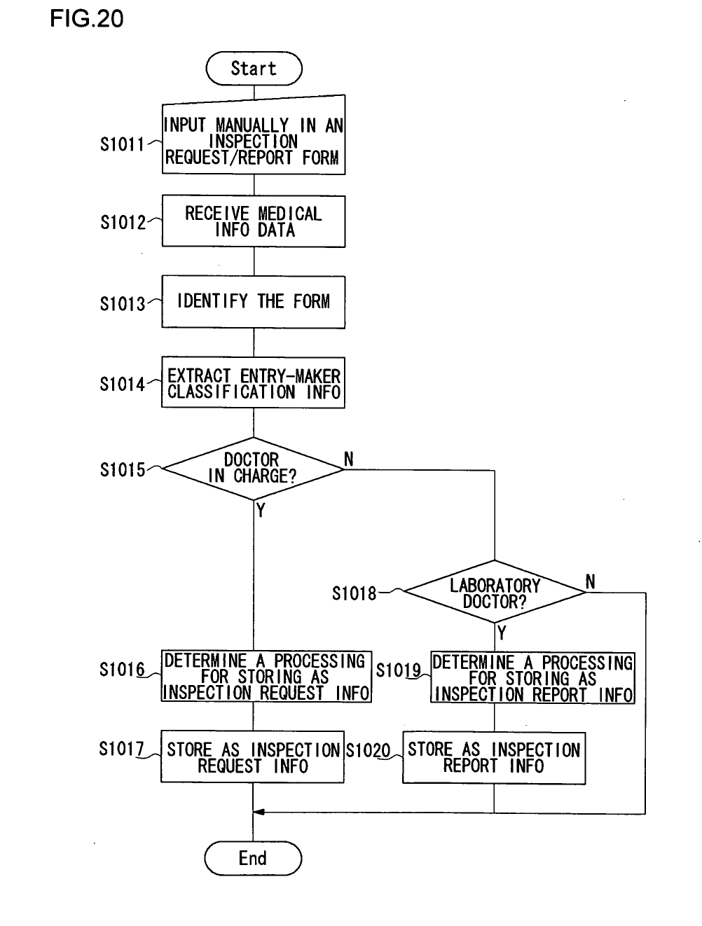

FIG. 20 is a flowchart showing the processing of an electronic patient's system according to the eighth system 1330.

First, the doctor in charge manually inputs medical information data onto an inspection request/report form 1302 using an electronic pen 1012 (S1011). As the medical information data are manually inputted onto the inspection request/report form with an electronic pen 1012, the electronic pen 1012 acquires the medical information data inputted manually onto the inspection request/report form, and transmits the acquired medical information data to a workstation 1200 in the division concerned. The workstation 1200 receives the medical information data transmitted from the electronic pen 1012. The workstation 1200 transmits the medical information data received from the electronic pen 1012 to the server 1100. The server 1100 receives the medical information data from the workstation 1200 via the network 1050 (S1012). In this manner, the server 1100 acquires the medical information data which were inputted in handwriting onto the inspection request/report form by the physician in charge.

As the server 1100 acquires the medical information data, a form identification unit 1132 extracts a from ID from the acquired medical information data and identifies a form 1010 entered with medical information represented by the medical information data by using the extracted form ID and a form table 1112 stored in a storage unit 1110 (S1013). As a result, a specific patient's inspection request/report form 1302 in which the medical information data have been manually inputted is identified, and form information corresponding to this inspection request/report form 1302 is identified out of the form information stored in the storage unit 1110. Also, the form identification unit 1132 identifies a form corresponding to this form information by identifying the form information. The received medical information data are associated with the identified form information and stored in the storage unit 1110.

As the inspection request/report form 1302 is identified, an entry-maker classification extraction unit 1136 extracts a pen ID from the acquired medical information data, and extracts entry-maker classification information from the acquired medical information data by using the extracted pen ID and an entry-maker classification table 1116 stored in the storage unit 1110 (S1014).

As the entry-maker classification information is extracted, a processing determination unit 1150 decides, by using the extracted entry-maker classification information, whether the person who had entered the medical information data in handwriting was the physician in charge or not (S1015). If it is determined that the person who had manually entered the medical information data was the physician in charge (Y of S1015), the processing determination unit 1150 will determine that the items entered in the inspection request/report form 1302 are the inspection request information and will then determine the processing of storing the acquired medical information data as the inspection request information (S1016). As the processing for storing the acquired medical information data as the inspection request information is selected, a registration processing unit 1164 stores the acquired medical information data in the storage unit 1110 as the inspection request information (S1017). More specifically, by appending inspection request identification data, indicating that the medical information data are inspection request data, to the medical information data, the registration processing unit 1164 stores the medical information data as inspection request data in the storage unit 1110. As a result, it becomes possible to effectively use the acquired medical information data as the inspection request information.

If it is determined that the person who had manually entered the medical information data was not the physician in charge (N of S1015), the processing determination unit 1150 will determine if the person who had manually entered the medical information was a laboratory doctor or not (S1018). If it is determined that the person who had manually entered the medical information data was not the laboratory doctor (N of S1018), the processing determination unit 1150 will determine that the acquired medical information data do not belong to neither inspection request information nor inspection report information and then complete the processing in the flowchart.

If it is determined that the person who had manually entered the medical information data was a laboratory doctor (Y of S1018), the processing determination unit 1150 will determine a processing for storing the acquired medical information data as the inspection report information (S1019). As the processing for storing it as the inspection report information is selected, the registration processing unit 1164 stores the acquired medical information data in the storage unit 1110 as the inspection report information (S1020). More specifically, by appending inspection report identification data, indicating that the medical information data are inspection report data, to the medical information data, the registration processing unit 1164 stores the medical information data as inspection report data in the storage unit 1110. As a result, it becomes possible to effectively use the acquired medical information data as the inspection report information.

If the physician in charge manually inputs the medical information data in any area, the medical information data will be stored as inspection request information in the storage unit 1110. If the physician in charge manually inputs the medical information data onto the inspection request/report form 1302, the medical information data will be stored as inspection report information in the storage unit 1110. Accordingly, even if the inspection request/report form 1302 differs in format for each facility or each inspection, the medical information data will be stored distinctively as either inspection request information or inspection report information, in the storage unit 1110.

Note that the processing determination unit 1150 may extract partial area information, indicating a partial area where the medical information data have been inputted, from the acquired medical information data. The processing determination unit 1150 may determine whether the medical information data are inputted in the first partial area 1402 or not, by using the extracted partial area information. If it is determined that the medical information data were inputted in the first partial area 1402, the processing determination unit 1150 may determine the processing for storing the medical information data as inspection request information in the storage unit 1110. If it is determined that the medical information data were inputted in the second partial area 1404, the processing determination unit 1150 may determine the processing for storing the medical information data as inspection report information in the storage unit 1110. As a result, the medical information data can be distinguished between the inspection request information and inspection report information, irrespectively of the entry-maker classification.

### Ninth Embodiment

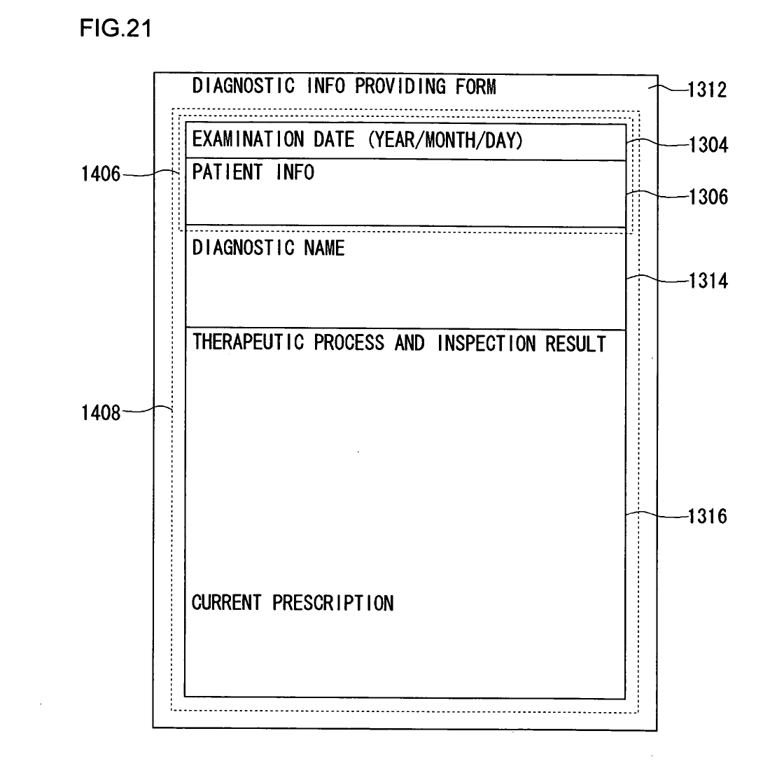

FIG. 21 illustrates a diagnostic information providing form 1312 used in an electronic patient's chart system 1300 according to a ninth embodiment. The diagnostic information providing form 1312 is prepared in such a manner that the form of a diagnostic information providing form 1312 is printed on a form 1010 already printed with a dot pattern which is readable by an electronic pen 1012. The diagnostic information providing form 1312 has an inspection date space 1304, a patient information space 1306, a diagnosis name space 1314 and a therapeutic process space 1316.

In the diagnostic information providing form 1312 like this, a laboratory doctor usually fills in all spaces of the inspection date space 1304, the patient information space 1306, diagnosis name space 1314 and therapeutic process space 1316. Of the medical information entered onto the diagnostic information providing form 1312, a third partial area containing the inspection date space 1304 and the patient information space 1306 is utilized in the medical accounting system. The medical information entered in a fourth partial area 1408 containing the inspection date space 1304, the patient information space 1306, the diagnostic name space 1314 and the therapeutic process space 1316 is utilized by a referral doctor. Hence, a technology is in demand that can perform in a further simplified manner the processing for communicating the medical information entered onto the diagnostic providing form 1312 to the medical accounting system and the processing for communicating it to the referral doctor by effectively utilizing the medical information data manually inputted onto the diagnostic information providing form 1312.

For that purpose, the electronic patient's chart system 1300 according to the ninth embodiment transmits medical information data, inputted in the third partial area 1406, out of the medical information data inputted manually onto the diagnostic information providing form 1312 to the medical accounting system. Also, the medical information data inputted in the fourth partial area 1408 is transmitted to the referral doctor.

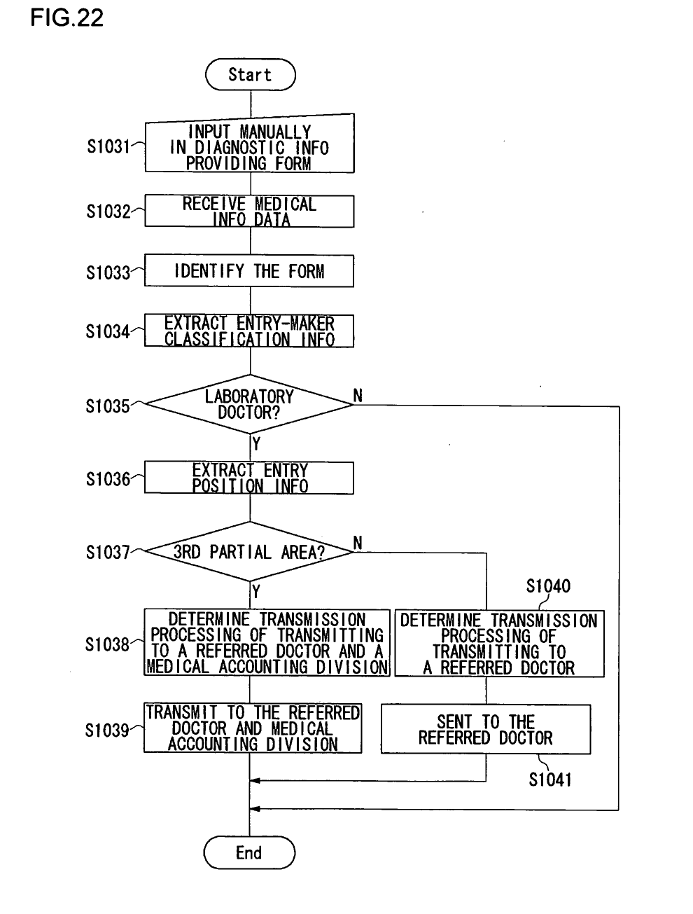

FIG. 22 is a flowchart showing the processing of an electronic patient's chart system 1300 according to the ninth embodiment.

First, a laboratory doctor enters diagnostic information onto a diagnostic information providing form 1312 using an electronic pen 1012 so as to input medical information data in handwriting (S1031). As the medical information data are manually inputted onto the diagnostic information providing form 1312 with an electronic pen 1012, the electronic pen 1012 acquires the medical information data inputted manually onto the diagnostic information providing form 1312, and transmits the acquired medical information data to a workstation 1200 in the division concerned. The workstation 1200 receives the medical information data transmitted from the electronic pen 1012. The workstation 1200 transmits the medical information data received from the electronic pen 1012 to the server 1100. The server 1100 receives the medical information data from the workstation 1200 via the network 1050 (S1032). In this manner, the server 1100 acquires the medical information data which were inputted in handwriting onto the diagnostic information providing form 1312 by the laboratory doctor.

As the server 1100 acquires the medical information data, the form identification unit 1132 extracts a from ID from the acquired medical information data and identifies a form 1010 entered with medical information represented by the medical information data by using the extracted form ID and the form table 1112 stored in the storage unit 1110 (S1033). As a result, a specific patient's diagnostic information providing form 1312 in which the medical information data have been manually inputted is identified, and form information corresponding to this diagnostic information providing form 1312 is identified out of the form information stored in the storage unit 1110. Also, the form identification unit 1132 identifies a form corresponding to this form information by identifying the form information. The received medical information data are associated with the identified form information and then stored in the storage unit 1110.

As the form information is identified, the entry-maker classification extraction unit 1136 extracts a pen ID from the acquired medical information data, and extracts entry-maker classification information from the acquired medical information data by using the extracted pen ID and the entry-maker classification table 1116 stored in the storage unit 1110 (S1034). As the entry-maker classification information is extracted, the processing determination unit 1150 decides whether the person who had entered the medical information data was the laboratory doctor or not, by using the extracted entry-maker classification information and the entry-maker classification table stored in the storage unit 1110 (S1035). If it is determined that the person who had manually entered the medical information data was not the laboratory doctor (N of S1035), the processing determination unit 1150 will determine that said medical information data shall not be transmitted to the outside and hence will terminate the processing in this flowchart. If it is determined that the person who had manually entered the medical information data was a laboratory doctor (Y of S1035), the entry position extraction unit 1138 will extract entry position information indicative of a position where the medical information has been manually inputted, from the acquired medical information data (S1036).

As the entry position information is extracted, a partial area extraction unit 1140 extracts a partial area where the acquired medical information has been manually inputted, using the extracted entry position information. Thereby, the processing determination unit 1150 determines whether the acquired medical data are manually inputted to the third partial area 1406 or not (S1037). This third partial area 1406 overlaps with the fourth partial area 1408. Thus, if it is determined that the acquired medical information data were manually inputted in the third partial area 1406 (Y of S1037), the processing determination unit 1150 will determine a processing for transmitting the medical information data to the referral doctor and the medical accounting division (S1038). As the processing performed on the medical information data is determined, the transmission processing unit 1162 transmits the medical information data to the referral doctor and the medical accounting division (S1039).

If it is determined that the acquired medical information data were not inputted manually in the third partial area 1406 (N of S1037), the processing determination unit11150 will determine a processing for transmitting the medical information data to the referral doctor (S1040). As the transmission processing for transmitting the medical information data to the referral doctor is determined, the transmission processing unit 1162 transmits the medical information data to the referral doctor (S1041). By carrying out the above steps, the medical information data indicative of medical information entered in the third partial area 1406 are transmitted to the referral doctor. As a result, an appropriate processing can be performed in accordance with a partial area where the medical information has been entered and thus the medical information data can be effectively utilized.

### Tenth Embodiment

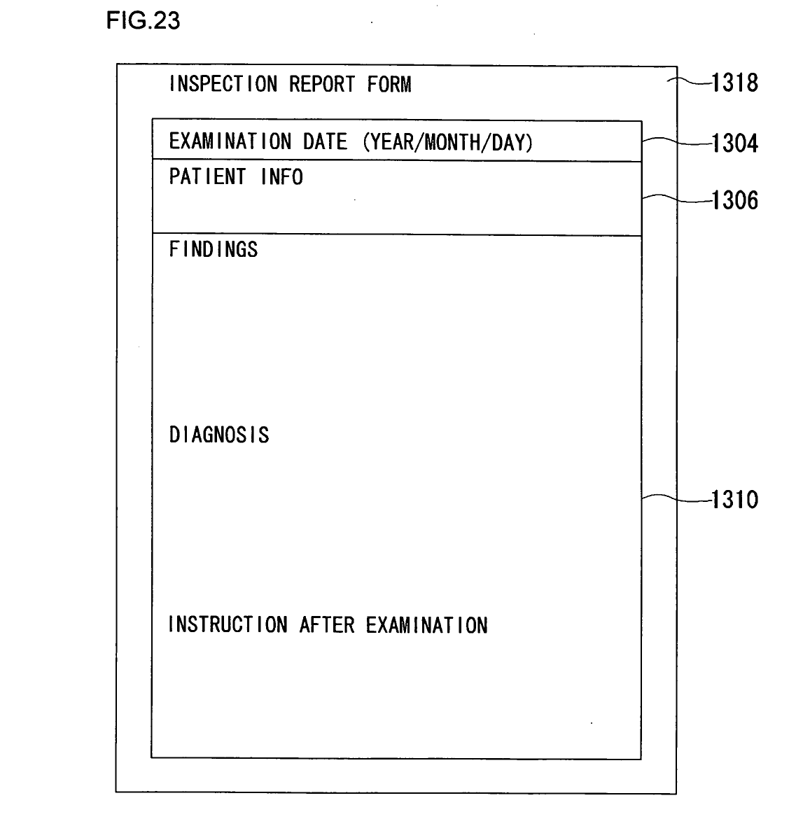

FIG. 23 illustrates an inspection report form 1318 used in an electronic patient's chart system 1300 according to a tenth embodiment. The inspection report form 1318 is prepared in such a manner that the form of an inspection report form 1318 is printed on a form 1010 already printed with a dot pattern which is readable by an electronic pen 1012. The inspection report form 1318 has an inspection date space 1304, a patient information space 1306, a finding/diagnosis space 1310.

A physician or medical doctor is normally the one who writes an inspection report on the inspection report form 1318. Note that the term “inspection” and “(medical) examination” are used interchangeably in this patent specification. The doctor meant here includes a trainee doctor who engages in the medical services and an advising doctor who supervises this trainee doctor. If, for instance, an inspection report is entered onto the inspection report form 1318 by a trainee doctor, the advising doctor will check the contents of this inspection report or add some comments thereto. Thus, when an inspection report is entered onto the inspection report form 1318 by a trainee doctor, the electronic patient's chart system 1300 first registers temporarily the medical information data indicative of the inspection report and then registers formally the medical information data indicative of the inspection report when the advising doctor later checks the contents of the inspection report or adds some comments thereto.

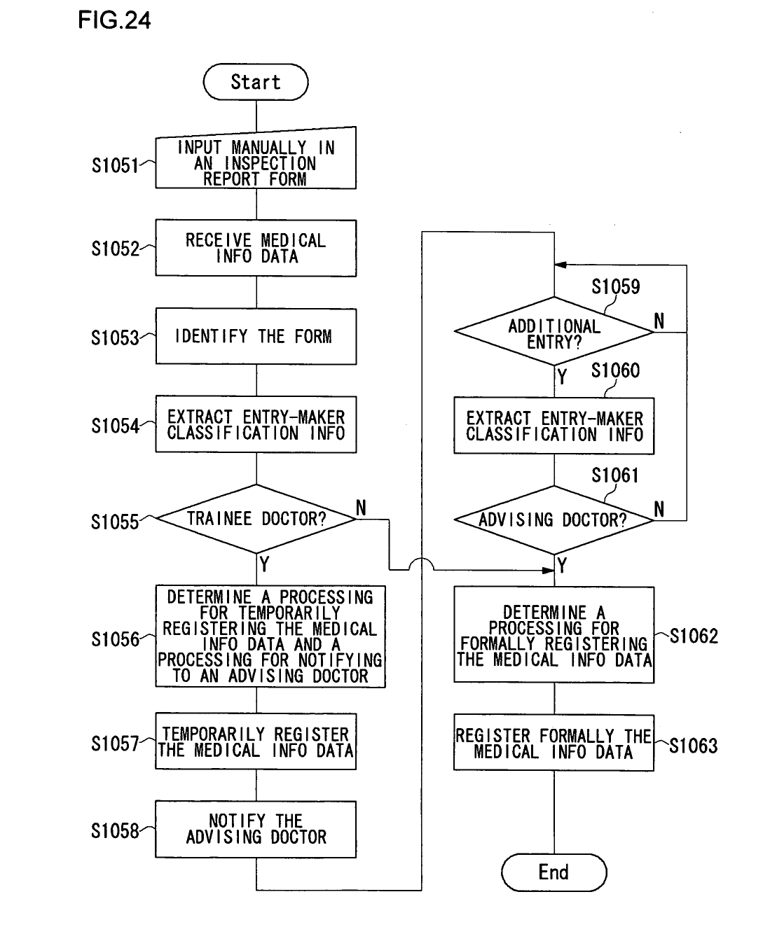

FIG. 24 is a flowchart showing the processing of an electronic patient's chart system 1300 according to the tenth embodiment.

First, a laboratory doctor enters an inspection report onto an inspection report form 1318 using an electronic pen 1012 so as to input medical information data in handwriting (S1051). As the medical information data are manually inputted onto the inspection report form 1318 with an electronic pen 1012, the electronic pen 1012 acquires the medical information data inputted manually onto the inspection report form 1318, and transmits the acquired medical information data to a workstation 1200 in the division concerned. The workstation 1200 receives the medical information data transmitted from the electronic pen 1012. The workstation 1200 transmits the medical information data received from the electronic pen 1012 to the server 1100. The server 1100 receives the medical information data from the workstation 1200 via the network 1050 (S1052). In this manner, the server 1100 acquires the medical information data which were inputted in handwriting onto the inspection report form 1318 by the trainee doctor.

As the server 1100 acquires the medical information data, the form identification unit 1132 extracts a from ID from the acquired medical information data and identifies a form 1010 onto which the medical information represented by the medical information data have been entered, by using the extracted form ID and the form table 1112 stored in the storage unit 1110 (S1053). As a result, a specific patient's inspection report form 1318 in which medical information data have been manually inputted is identified, and form information corresponding to this inspection report form 1318 is identified out of the form information stored in the storage unit 1110. Also, the form identification unit 1132 identifies a form corresponding to this form information by identifying the form information. The received medical information data are associated with the identified form information and then stored in the storage unit 1110. As the form information is identified, the entry-maker classification extraction unit 1136 extracts a pen ID from the acquired medical information data, and extracts entry-maker classification information from the acquired medical information data by using the extracted pen ID and the entry-maker classification table 1116 stored in the storage unit 1110 (S1054).

As the entry-maker classification information is extracted, the processing determination unit 1150 decides, by using the extracted entry-maker classification information, whether the person who had entered the medical information data was trainee doctor or not (S1055). If it is determined that the person who had manually entered the medical information data was not trainee doctor (N of S1055), the processing determination unit 1150 will determine that the person who had entered the medical information data was the advising doctor and then skip the step of temporarily registering the medical information data and move on to Step S1062.

If it is determined that the person who had manually entered the medical information data was a trainee doctor (Y of S1055), the processing determination unit 1150 will determine the execution of a processing for referring to the data table 1118 and temporarily registering the medical data and a processing for informing to an advising doctor (S1056). As a processing which is executed on the medical information data is determined, the registration processing unit 1164 first registers the medical information data temporarily (S1057). The registration processing unit 1164 temporarily registers the medical information data in a manner that temporary identification data indicative of a temporary registration are appended to the medical information data so as to be stored in the storage unit 1110.

In the state where the medical information data are being registered temporarily, the user cannot display the temporarily registered medical information data on a display and cannot print them either. The output processing unit 1166 of the server 1100 and the output processing unit 1218 of the workstation 1200 carry out the output processing where the temporarily registered medical information data are not displayed on a display and the output processing where those are not printed by a printer. The output processing unit of the server 1100 or the output processing unit 1218 of the workstation 1200 may display the temporarily registered medical information data in such a manner that the temporarily registered medical information data can be distinguished visually from the formally registered medical information data by varying a display mode such as color. If the temporarily registered medical information data are to be displayed on a display, the characters “temporarily registered” or the like may be displayed together. Thereby, the user who browses the form information representing the inspection report form 1318 can recognize that the medical information data are in the state of being temporarily registered.

As the medical information data are temporarily registered, the transmission processing unit 1162 notifies the advising doctor the of information that the inspection report data have been entered onto the inspection report form 1318 by the trainee doctor (S1058). The inspection report form 1318 onto which the inspection report data have been entered by the inspection doctor is delivered to the advising doctor. By looking at this notification, the advising doctor can recognize that the inspection report data had been entered onto the inspection report form 1318 by the trainee doctor

As the notification to the advising doctor is carried out, the processing determination unit 1150 decides whether any additional entry has been made in the inspection report form 1318 or not (S1059). When no additional entry has been made in the inspection report form 1318 (N of S1059), the processing determination unit 1150 keeps the state where the medical information data are temporarily registered, until an additional entry is made again. When an additional entry has been made in the inspection report form 1318 (Y of S1059), the entry-maker classification extraction unit 1136 extracts a pen ID from the acquired medical information data, and extracts entry-maker classification information from the acquired medical information data by use of the extracted pen ID and the entry-maker classification table 1116 stored in the storage unit 1110 (S1060)

As the entry-maker classification information is extracted, the processing determination unit 1150 decides, by using the extracted entry-maker classification information, whether the person who had entered the medical information data in handwriting was the advising doctor or not (S1061). If it is determined that the person who had manually entered the medical information data was not the advising doctor (N of S1061), the processing determination unit 1150 will keep the state where the medical information data are temporarily registered, until an additional entry is made again. If it is determined that the person who had manually entered the medical information data was the advising doctor (Y of S1061), the processing determination unit 1150 will refer to the data processing table 1118 and determine a processing for formally registering the medical information data (S1062). As the processing for formally registering the medical information data is decided on, the registration processing unit 1164 registers formally the medical information data (S1063). The registration processing unit 1164 registers formally the medical information data in a manner that formal registration identification data indicative of being formally registered are added to the medical information data and then stored in the storage unit 1110. The formal registration of medical information data may be done in such a manner that the registration processing unit 1164 removes the temporary registration identification data, indicative of being temporarily registered, from the acquired medical information data.

In the state where the medical information data are being formally registered, if, for instance, any of users browses the form information representing this inspection report form 1318, from the workstation 1200, the user can display the formally registered medical information data on a display and can also print them. The output processing unit 1166 of the server 1100 and the output processing unit 1218 of the workstation 1200 carry out the output processing so that the formally registered medical information data are displayed on a display and they are printed by a printer. With this structure and its operations as described above, the advising doctor can accurately and promptly check the inspection report form 1318 onto which the inspection report data have been entered by the advising doctor and trainee doctor, by utilizing effectively the medical information data.

### Eleventh Embodiment

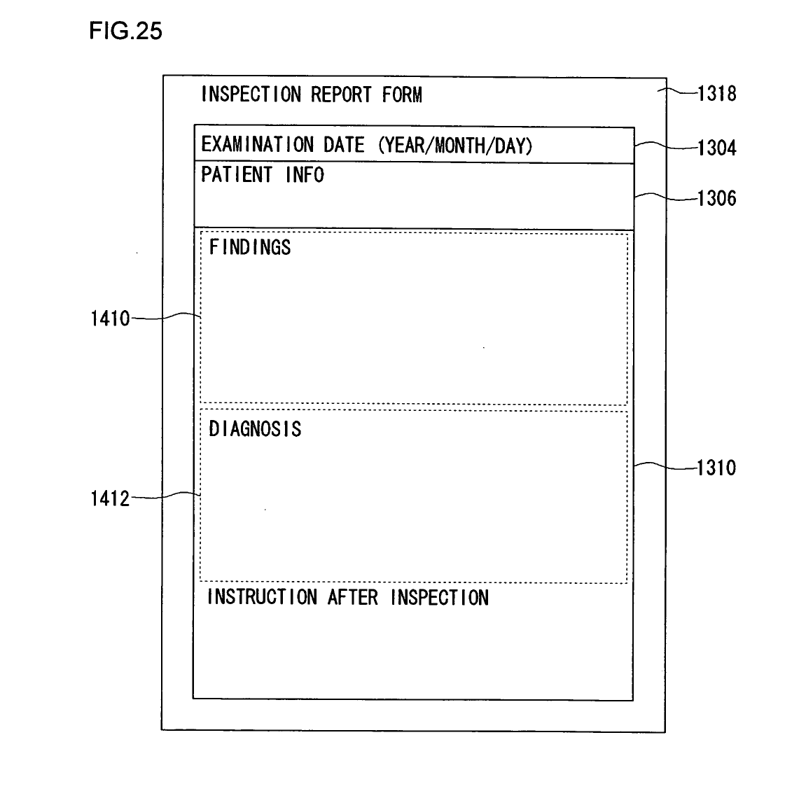

FIG. 25 illustrates an inspection report form 1318 used in an electronic patient's chart system 1300 according to an eleventh embodiment. The inspection report form 1318 is prepared in such a manner that the form of an inspection report form 1318 is printed on a form 1010 already printed with a dot pattern which is readable by an electronic pen 1012. The inspection report form 1318 has an inspection date space 1304, a patient information space 1306 and a finding/diagnosis space 1310. The finding/diagnosis space 1310 has a fifth partial area 1410 as a partial area where a physician's opinion is entered and a sixth partial area 1412 as a partial area where the diagnosis is entered.

A physician or medical doctor is normally the one who writes a medical report on the inspection report form 1318. However, there are cases where a laboratory technician who conducts an inspection enters findings onto the inspection report form 1318. The laboratory technician meant here is a person who is licensed to conduct an inspection or examination related to medical services under the supervision of a physician. The laboratory technician may enter findings onto the inspection report form 1318 but may not enter the diagnosis. Thus, a new technology is in demand where a diagnostician or medical examiner can promptly and accurately check the findings entered by laboratory technicians, by utilizing effectively the medical information data manually inputted onto the inspection report form 1318.

In the electronic patient's chart system 1300 according to the eleventh embodiment, if medical information data are manually inputted in the fifth partial area 1410, the medical information data will be registered temporarily. If a laboratory technician manually inputs in the fifth partial area 1410, the electronic patient's chart system 1300 will keep the state of being temporarily registered, until the confirmation or addition is made by the diagnostician. If the medical data are manually inputted, by a diagnostician, in the sixth partial area 1412 serving as the partial area where a diagnosis is to be entered, the electronic patient's system 1300 will register the medical information data formally.

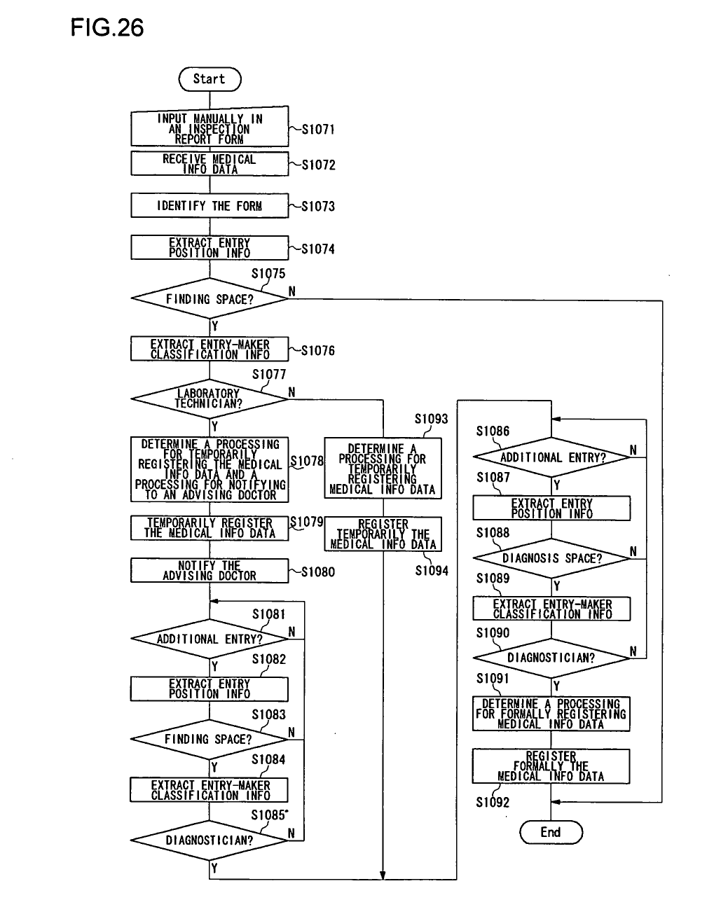

FIG. 26 is a flowchart showing the processing of an electronic patient's chart system 1300 according to the eleventh embodiment.

First, a diagnostician or a laboratory technician enters an inspection report onto an inspection report form 1318 using an electronic pen 1012 so as to input medical information data in handwriting (S1071). As the medical information data are manually inputted onto the inspection report form 1318 with an electronic pen 1012, the electronic pen 1012 acquires the medical information data inputted manually onto the inspection report form 1318, and transmits the acquired medical information data to a workstation 1200 in the division concerned. The workstation 1200 receives the medical information data transmitted from the electronic pen 1012. The workstation 1200 transmits the medical information data received from the electronic pen 1012 to the server 1100. The server 1100 receives the medical information data from the workstation 1200 via the network 1050 (S1072). In this manner, the server 1100 acquires the medical information data which were inputted in handwriting onto the inspection report form 1318 by the diagnostician or laboratory technician.

As the server 1100 acquires the medical information data, the form identification unit 1132 extracts a from ID from the acquired medical information data and identifies a form 1010 entered with medical information represented by the medical information data by using the extracted form ID and the form table 1112 stored in the storage unit 1110 (S1073). As a result thereof, an inspection report form 1318 in which the medical information data have been manually inputted is identified, and form information corresponding to this inspection report form 1318 is identified out of the form information stored in the storage unit 1110. Also, the form identification unit 1132 identifies a form corresponding to this form information by identifying the form information. The received medical information data are associated with the identified form information and then stored in the storage unit 1110.

As the form information is identified, the entry position extraction unit 1138 extracts the entry position of the acquired medical information data (S1074). As the partial area information is extracted, the partial area extraction unit 1140 extracts the partial area information indicative of a partial area in which the medical information data have been manually inputted, by using the extracted entry position information. By determining whether or not the extracted partial area information is indicative of the fifth partial area 1410 utilized as a partial area for inputting findings, the processing determination unit 1150 determines if the position at which the acquired medical information data have been manually inputted is in the finding space or not (S1075).

If it is determined that the position at which the acquired medical information data have been manually inputted is not in the finding space (N of 1075), the processing determination unit 1150 will skip the steps of temporarily registering and formally registering the medical information data, and will then complete the processing in this flowchart. If it is determined that the position at which the acquired medical information data have been manually inputted is in the finding space (Y of 1075), the entry-maker classification extraction unit 1136 will extract a pen ID from the acquired medical information data and extract entry-maker classification information from the acquired medical information by using the extracted pen ID and the entry-maker classification table 1116 stored in the storage unit 1110 (S1076)

As the entry-maker classification information is extracted, the processing determination unit 1150 decides, by using the extracted entry-maker classification information, whether the person who had entered the medical information data was laboratory doctor or not (S1077). If it is determined that the person who had manually entered the medical information data was a laboratory doctor (Y of S1077), the processing determination unit 1150 will refer to the data processing table 1118 and determine a processing for temporarily registering the medical information data and a processing for notifying to a diagnostician (S1078). As the processing for temporarily registering the medical information data and the processing for notifying to a diagnostician are determined, the registration processing unit 1164 will register the medical information data temporarily (S1079). The processing for temporarily registering the medical information data and the output processing for displaying or printing an output in the case of the temporary registration of the medical information data are the same as those described in the tenth embodiment.

As the medical information data are registered temporarily, the transmission processing unit 1162 communicates to the diagnostician to the effect that the finding was entered onto the inspection report form 1318 by a laboratory technician (S1080). The inspection report form 1318 onto which the finding was entered by the laboratory doctor is delivered to the diagnostic doctor. By looking at this notification, the diagnostician can recognize that the inspection report form 1318 delivered was filled in, with the findings, by an laboratory technician.

If it is determined that the person who had manually entered the medical information data was not laboratory doctor (N of S1077), the processing determination unit 1150 will refer to the data processing table 1118, determine that the person who had manually entered the medical information data was a diagnostician and then determine a processing for temporarily registering the medical information data (S1093). As the processing for temporarily registering the medical information data is determined, the registration processing unit 1164 registers the medical information data temporarily the same way as the above-described (S1094). Now, when the medical information data have been temporarily registered, the processing determination unit 1150 will shift the processing to S1086 so as to formally register the medical information data.

As the notification to the diagnostician is carried out, the processing determination unit 1150 decides whether any additional entry has been made in the inspection report form 1318 or not (S1081). When no additional entry has been made (N of S1081), it keeps the state where the medical information data are temporarily registered, until an additional entry is made, so that it does not shift to the processing for formally registering the medical information data. When an additional entry has been made (Y of S1081), the entry position extraction unit 1138 extracts the entry position information on the acquired medical information data (S1082).

As the entry position information is extracted, the partial area extraction unit 1140 extracts the partial area information indicative of a partial area in which the medical information data have been manually inputted, using the extracted entry position information. As a result, the processing determination unit 1150 decides whether the position at which the acquired medical information data have been manually inputted is in the finding space or not (S1083). If it is determined that the space at which the medical information data have been manually inputted is not in the finding space (N of S1083), the processing determination unit 1150 will keep the state where the medical information data are temporarily registered, until an additional entry is made again, and thus will not proceed to a processing for normally registering the medical information data.

If it is determined that the space at which the medical information data have been manually inputted is in the finding space (Y of S1083), the entry-maker classification extraction unit 1136 will extract a pen ID from the acquired medical information data and extract entry-maker classification information from the acquired medical information data by using the extracted pen ID and an entry-maker classification table 1116 stored in the storage unit 1110 (S1084). As the entry-maker classification information is extracted, the processing determination unit 1150 decides whether the person who had manually entered the medical information data was diagnostician or not, by using the extracted entry-maker classification information (S1085). If it is determined that the space at which the medical information data have been manually inputted is not the finding space (N of S1085), the state where the medical information data are temporarily registered will be kept until an additional entry is made again and the step will not proceed to the processing for normally registering the medical information data. Thereby, in the case where the findings are entered by a laboratory technician, registering formally the medical information data without the confirmation by the diagnostician can be prevented.

If it is determined that the person who had manually entered the medical information data is a diagnostician and the findings entered by the laboratory technician is checked by the diagnostician (Y of S1085), the processing determination unit 1150 will proceed the processing to S1086 so as to formally register the medical information data.

In the state where the medical information data are being temporarily registered, the processing determination unit 1150 decides whether any additional entry has been made in the inspection report form 1318 or not (S1086). When no additional entry has been made in the inspection report form 1318 (N of S1086), the state where the medical information data are temporarily registered is kept until an additional entry is made. When any additional entry has been made (Y of S1086), an entry position extraction unit 1138 extracts entry position information from the medical information data indicative of medical information acquired as a result of the addition (S1087).

As the entry position information is extracted, the partial area extraction unit 1140 extracts partial area information indicative of a partial area in which the medical information data have been manually inputted, by using the acquired entry position information. By determining whether the extracted partial area information is indicative of a sixth partial area 1412 or not, the processing determination unit 1150 determines whether any additional entry has been made onto the diagnosis space or not (S1088). If it is determined that no additional entry has been entered onto the diagnosis space (N of S1088), the state in which the medical information data are temporarily registered is kept until an additional entry is made again.

If it is determined that any additional entry has been made in the diagnosis space (Y of S1088), the entry-maker classification extraction unit 1136 extracts a pen ID from the acquired medical information data and extracts the entry-maker classification information from the acquired medical information data by using the extracted pen ID and the entry-maker classification table 1116 stored in the storage unit 1110 (S1089). As the entry-maker classification information is extracted, the processing determination unit 1150 determines, by using the extracted entry-maker classification information, whether the person who had manually entered the medical data is a diagnostician or not (S1090). If it is determined that the person who had manually entered the medical information data was not a diagnostician (N of S1090), the state in which the medical information data are temporarily registered will be kept until an additional entry is made again. In this manner, the state in which the medical information data are temporarily registered is kept until the diagnosis is entered by the diagnostician, so that registering formally the medical information data before the diagnosis is entered by the diagnostician can be prevented.

If it is determined that the person who had manually entered the medical information data is a diagnostician (Y of S1090), the processing determination unit 1150 will refer to the data processing table 1118, determine that the diagnosis has been entered by the diagnostician and therefore determine a processing for formally registering the medical information data (S1091). When the processing for formally registering the medical information is decided on, the registration processing unit 1164 registers formally the medical information data (S1092). The processing for formally registering the medical information data and the output processing for displaying or printing an output in the case of the formal registration of the medical information data are the same as those described in the eleventh embodiment. By employing the structure and method according to the eleventh embodiment, the diagnostician can accurately and promptly check the inspection report form 1318 onto which the findings have been entered by the laboratory technician, by utilizing effectively the medical information data.

### Twelfth Embodiment

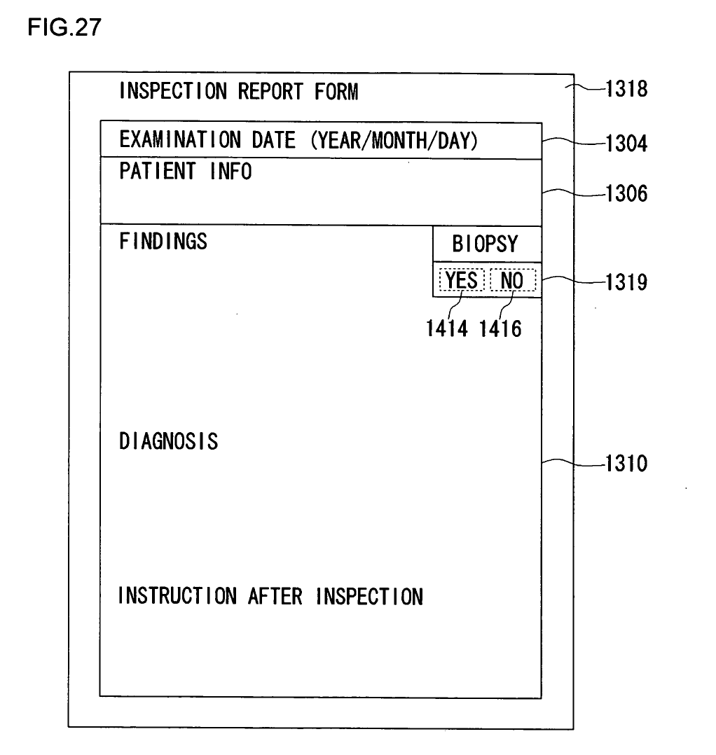

FIG. 27 illustrates an inspection report form 1318 used in an electronic patient's chart system 1300 according to a twelfth embodiment. The inspection report form 1318 is prepared in such a manner that the form of an inspection report form 1318 is printed on a form 1010 already printed with a dot pattern which is readable by an electronic pen 1012. The inspection report form 1318 has an inspection date space 1304, a patient information space 1306, a finding/diagnosis space 1310 and an eighth partial area 1416. A biopsy status space 1319 has a seventh partial area 1414 as a partial area where a check, a circle or the like is placed in the case when a biopsy is done and an eighth partial area 1416 as a partial area where a check, a circle or the like is placed in the case when no biopsy is done.

For example, there are cases where a biopsy is performed in which a tissue of polyp or the like is taken in an endoscopic inspection. When the biopsy is performed, the tissue obtained like this is sent to a pathology laboratory division so as to carry out a pathological examination thereon. With a result of this pathological examination, it becomes possible to determine, for example, whether or not a cancer cell is found from the tissue taken. In this manner, when a biopsy is performed, a physician can determine or advise properly that, for example, the result of diagnosis is cancer and an operation is advised after the examination and so forth, only when he/she is informed of the result of pathological examination on the tissue taken by the biopsy. Hence, a new technology is in demand which allows the pathological laboratory division to grasp quickly and accurately a pathological examination request by effectively using the medical information data manually entered onto the inspection report form 1318 when the biopsy is performed. In a case when the biopsy is done, the electronic patient's chart system 1300 according to the twelfth embodiment registers the medical data information entered manually onto the inspection report form 1318, as a tentative report. Then, a pathological examination is requested of the pathological laboratory division. When the result of a pathological examination is inputted, the medical information data entered manually onto the inspection report form 1318 is registered as a final report.

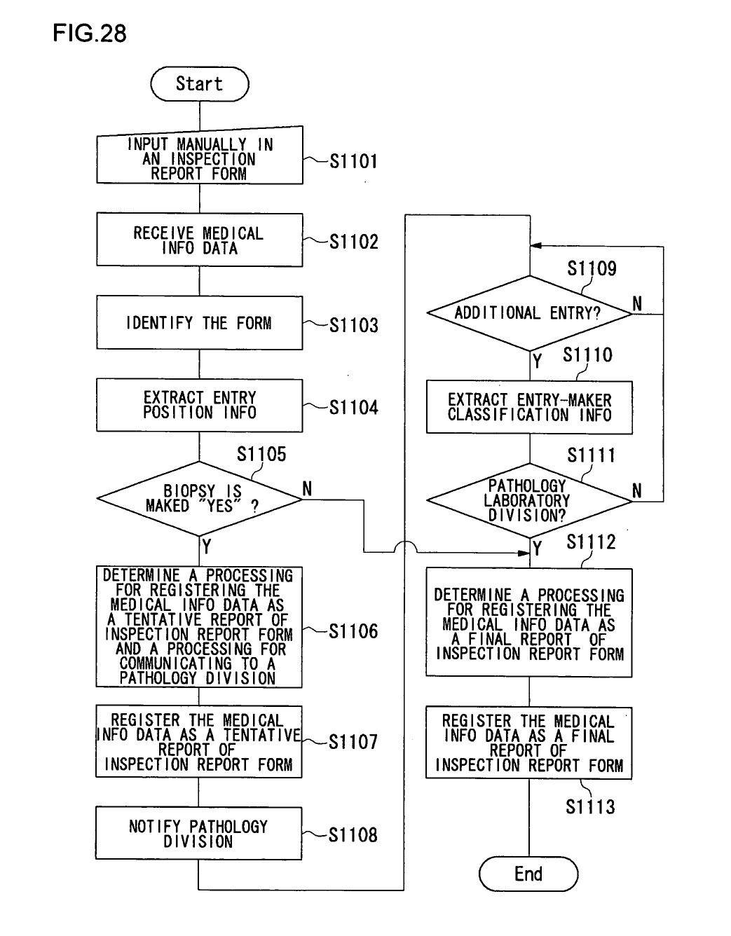

FIG. 28 is a flowchart showing the processing of an electronic patient's chart system 1300 according to the twelfth embodiment.

First, a laboratory doctor enters an inspection report onto an inspection report form 1318 using an electronic pen 1012 so as to input medical information data in handwriting (S1101). As the medical information data are manually inputted onto the inspection report form 1318 with an electronic pen 1012, the electronic pen 1012 acquires the medical information data inputted manually onto the inspection report form 1318, and transmits the acquired medical information data to a workstation 1200 in the division concerned. The workstation 1200 receives the medical information data transmitted from the electronic pen 1012. The workstation 1200 transmits the medical information data received from the electronic pen 1012 to the server 1100. The server 1100 receives the medical information data from the workstation 1200 via the network 1050 (S1102). In this manner, the server 1100 acquires the medical information data which were inputted in handwriting onto the inspection report form 1318 by the laboratory doctor.

As the server 1100 acquires the medical information data, the form identification unit 1132 extracts a from ID from the acquired medical information data and identifies a form 1010 entered with medical information represented by the medical information data by using the extracted form ID and the form table 1112 stored in the storage unit 1110 (S1103). As a result thereof, an inspection report form 1318 in which the medical information data have been manually inputted is identified, and form information corresponding to this inspection report form 1318 is identified out of the form information stored in the storage unit 1110. Also, the form identification unit 1132 identifies a form corresponding to this form information by identifying the form information. The received medical information data are associated with the identified form information and then stored in the storage unit 1110.

As the form information is identified, the entry position extraction unit 1138 extracts the entry position of the acquired medical information data (S1104). The partial area extraction unit 1140 extracts the partial area information indicative of a partial area in which the medical information data have been manually inputted, by using the extracted entry position information. By determining whether or not the extracted partial area information is indicative of the seventh partial area 1414 serving as a partial area where a check is placed on “YES” in the biopsy status space, the processing determination unit 1150 determines if a check is placed on “YES” in the biopsy status space or not (S1105). If a check is not placed on “YES” in the biopsy status space (N of S1105), the processing determination unit 1150 will register the medical information data as the final report since the pathological examination is conducted, so that the processing is shifted to S1112.

If a check is placed on “YES” in the biopsy status space (Y of S1105), the processing determination unit 1150 will refer to the data processing table 1118 and determine a processing for registering the medical information data as a tentative report of the inspection report form and a processing for notifying the pathology laboratory division (S1106). As a processing performed on the medical information data is determined, the registration processing unit 1164 registers the medical information data as tentative report data of the inspection report form (S1107). The registration processing unit 1164 registers the medical information data as tentative report data in a manner that tentative report identification data indicative of being tentative report data are appended to the medical information data so as to be stored in the storage unit 1110.

In the state where the medical information data are being registered as tentative or interim report data, suppose, for instance, that any of users browses the form information representing this inspection report form 1318, from the workstation 1200. Then the medical information data registered as tentative report data are displayed on a display in a manner that can be distinguished visually from the medical information data registered as final report data, by varying a display mode such as color. The output processing unit 1166 of the server 1100 and the output processing unit 1218 of the workstation 1200 carry out the output processing so that the medical information data registered as tentative report data are displayed on a display in a manner that can be distinguished visually from the medical information data registered as final report data. Also, the output processing unit 1166 and the output processing unit 1218 carry out the output processing so that the medical information data registered as tentative report data are printed by a printer in a manner that can be distinguished visually from the medical information data registered as final report data. When the medical information data registered as tentative report data are displayed on a display, the characters “tentative report” or the like may be displayed together. Thereby, the user who browses the form information representing the inspection report form 1318 can recognize that the medical information data are in the state of being registered as tentative report data.

As the medical information data are registered as tentative report data of the inspection report form, the transmission processing unit 1162 communicates to the pathology laboratory division to the effect that a biopsy was performed (S1108). In this case, the inspection report form 1318, the tissue taken by biopsy and so forth are delivered to the pathology laboratory division. With those delivered thereto, the pathology laboratory division can promptly recognize that a biopsy has been performed and a pathological examination needs to be conducted, by using effectively the medical data information, so that a necessary medical action can be taken quickly and accurately.

As the above notification is made to the pathology laboratory division, the processing determination unit 1150 determines whether any additional entry has been made onto the inspection report form 1318 or not (S1109). The decision on whether any additional entry has been made to the inspection report form 1318 or not is made the same way as the previous embodiments.

If no additional entry is made onto the inspection report form 1318 (N of S1109), the state in which the medical information data are registered as tentative report data will be kept until an additional entry is made. When an additional entry is made (Y of S1109), the entry-maker classification extraction unit 1136 extracts a pen ID from the acquired medical information data and extracts the entry-maker classification information from the acquired medical information data by using the extracted pen ID and the entry-maker classification table 1116 stored in the storage unit 1110 (S1110).

As the entry-maker classification information is extracted, the processing determination unit 1150 determines, by using the extracted entry-maker classification information, whether the person who had manually entered the medical data is a person belonging to the pathology laboratory division or not (S1111). If it is determined that the person who had manually entered the medical information data was not a person belonging to the pathology laboratory division (N of S1111), the processing determination unit 1150 will keep the state in which the medical information data are registered as tentative report data, until an additional entry is made again. In this manner, the processing determination unit 1150 keeps the state in which the medical information data are registered as tentative report data if the input of a result of a pathological examination or a correction input in response to the result of a pathological examination is not made from the pathology laboratory division. By employing the structure and method described as above, registering the medical information data as final report data can be prevented if no such input was made from the pathology laboratory division.

If it is determined that the person who had manually entered the medical information data was a person belonging to the pathology laboratory division (Y of S1111), the processing determination unit 1150 will refer to the data processing table 1118, determine that the input of a result of a pathological examination has been made or a correction input of medical information data of the inspection report form 1318 in response to the result of a pathological examination has been made from the pathology laboratory division, and therefore determine a processing for registering the medical information data as the final report data of the inspection report form (S1112). As a processing performed on the medical information data is determined, the registration processing unit 1164 registers the medical information data as final report data (S1113). The registration processing unit 1164 registers the medical information data as final report data in a manner that final report identification data indicative of being final report data is appended to the medical information data so as to be stored in the storage unit 1110. Note that the registration processing unit 1164 may register the medical information data as final report data by deleting the tentative report identification data. By employing the structure and the method described as above, the electronic patient's chart system 1300 according to the twelfth embodiment allows the pathological laboratory division to verify quickly and accurately a pathological examination request by effectively using the medical information data when the biopsy is performed.

### Thirteenth Embodiment

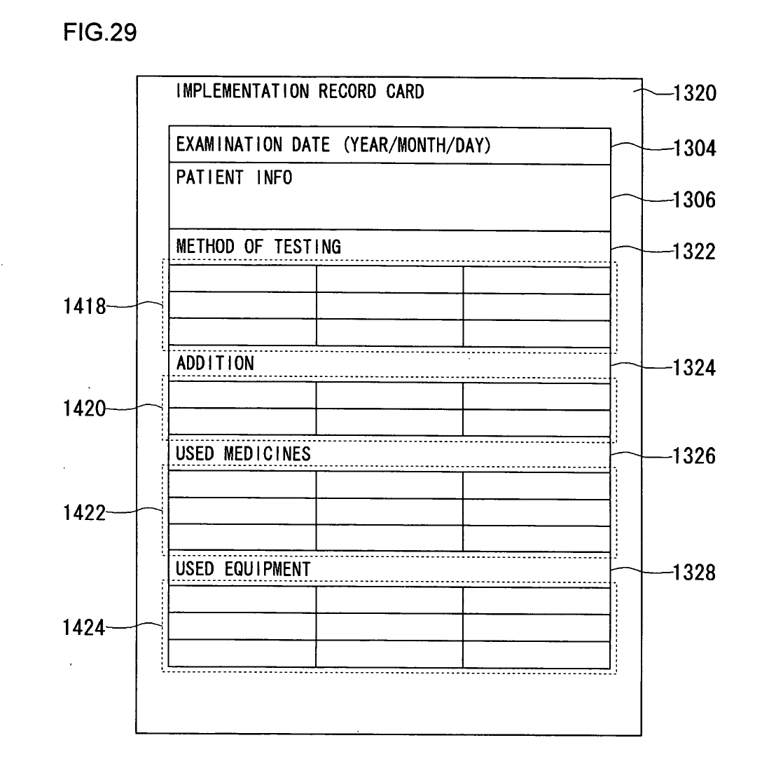

FIG. 29 illustrates an implementation record card 1320 used in an electronic patient's chart system 1300 according to a thirteenth embodiment. The implementation record card 1320 is prepared in such a manner that the form of an inspection report form 1318 is printed on a form 1010 already printed with a dot pattern which is readable by an electronic pen 1012. The implementation record card 1320 has an inspection date space 1304, a patient information space 1306, a method-of-testing space 1322, an add space 1324, a used medicines space 1326 and an used equipment space 1328. The method-of-testing space 1322 has a ninth partial area 1418 as a partial area where the items or list of laboratory techniques are entered. The add space 1324 has a tenth partial area 1420 as a partial area where the add items are entered. The used medicines space 1326 has an eleventh partial area 1422 as a partial area where the used medicines are entered. The used equipment space 1328 has a twelfth partial area 1424 as a partial area where the used equipment is entered.

A laboratory doctor or a nurse enters all the medical actions conducted, onto the implementation record card 1320. In the implementation record card 1320 used in the electronic patient's chart system 1300 according to the thirteenth embodiment, the laboratory doctor and the nurse enter the items of laboratory techniques conducted, in the ninth partial area 1418 and they enter the add items conducted, in the tenth partial area 1420. The laboratory doctor and the nurse enter the used medicines, in the eleventh partial space 1422 and they enter the used equipment, in the twelfth partial area 1424.

In such a case, there are cases where suppose that there are two clinical cases and two different treatments are provided, then only one of the two treatments provided can be counted as insurance points. In this case, a staff in the medical accounting division, for example, enters a mark in an item to be counted as an insurance point in each of items entered onto the implementation record card 1320, so that a clear distinction can be made between the items to be counted as insurance points and those not to be counted as insurance points. However, in this method for only entering a mark in the implementation record card 1320, the staff in the medical accounting division has to carry out the medical accounting processing while verifying the contents of marked implementation and therefore the staff may overlook some marks. Accordingly, a new technology is in demand where the medical accounting processing can be performed in a further simplified and accurate manner by utilizing effectively the medical information data entered onto the implementation record card 1320.

In the light of this, the electronic patient's chart system 1300 according to the thirteenth embodiment stores the medical information data, indicative of the implementation contents entered onto the implementation record card 1320, in the storage unit 1110. By identifying the medical information data indicative of the implementation contents in which the marks have been entered in the items to be counted as insurance points by the staff members in the medical accounting division, the electronic patient's chart system 1300 performs the accounting processing on only the implementation contents in which the marks have been placed.

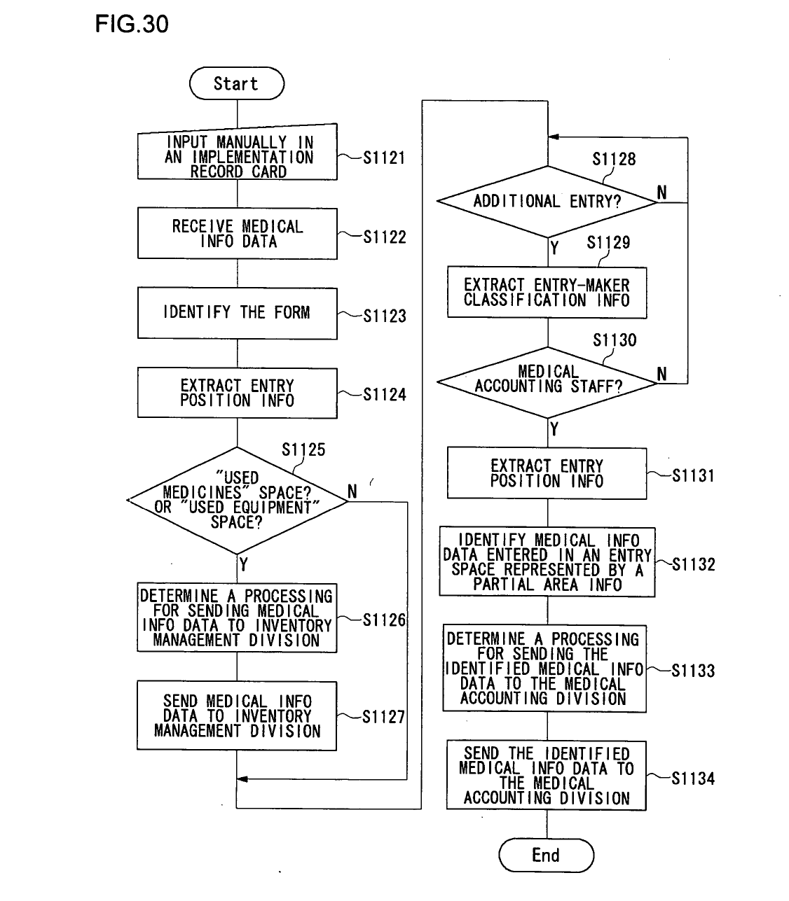

FIG. 30 is a flowchart showing the processing of an electronic patient's chart system 1300 according to the thirteenth embodiment. First, a laboratory doctor and a nurse enter implementation contents, marks or the like onto an implementation record card 1320, using an electronic pen 1012 so as to input medical information data in handwriting (S1121). As the medical information data are manually inputted onto the implementation record card 1320 with an electronic pen 1012, the electronic pen 1012 acquires the medical information data inputted manually onto the implementation record card 1320, and transmits the acquired medical information data to a workstation 1200 in the division concerned. The workstation 1200 receives the medical information data transmitted from the electronic pen 1012. The workstation 1200 transmits the medical information data received from the electronic pen 1012 to the server 1100. The server 1100 receives the medical information data from the workstation 1200 via the network 1050 (S1122). In this manner, the server 1100 acquires the medical information data which were inputted in handwriting onto the implementation record card 1320 by the laboratory doctor and the nurse.

As the server 1100 acquires the medical information data, the form identification unit 1132 extracts a from ID from the acquired medical information data and identifies a form 1010 entered with medical information represented by the medical information data by using the extracted form ID and the form table 1112 stored in the storage unit 1110 (S1123). As a result thereof, an implementation record card 1320 in which the medical information data have been manually inputted is identified, and form information corresponding to this implementation record card 1320 is identified out of the form information stored in the storage unit 1110. Also, the form identification unit 1132 identifies a form corresponding to this form information by identifying the form information. The received medical information data are associated with the identified form information and then stored in the storage unit 1110.

As the form information is identified, the entry position extraction unit 1138 extracts the entry position of the acquired medical information data (S1124). The partial area extraction unit 1140 extracts the partial area information indicative of a partial area in which the medical information data have been manually inputted, by using the extracted entry position information. By determining whether or not the extracted partial area information is indicative of the eleventh partial area 1422 or the twelfth partial area 1424, the processing determination unit 1150 determines if the position at which the acquired medical information data have been manually inputted is in the used medicines space 1326 or the used equipment space 1328 (S1125).

If it is determined that the position at which the acquired medical information data have been manually inputted is in neither the used medicines space 1326 nor the used equipment space 1328 (N of 1125), the processing determination unit 1150 will determine that there is no need to transmit the medical information data to an inventory management personnel and therefore it will skip the step of transmitting the medical information data to the inventor management personnel. If it is determined that the position at which the acquired medical information data have been manually inputted is in the used medicines space 1326 or the used equipment space 1328 (Y of 1125), the processing determination unit 1150 will refer to the data processing table 1118 and determine a processing for transmitting the medical information data to an inventory management division (S1126). As the processing for transmitting the medical information data to the inventory management division is determined, the transmission processing unit 1162 transmits the medical information data to the inventory management division (S1127). As a result, the medical information data manually entered onto the implementation record card 1320 can be utilized effectively for managing the inventory of the medicines and equipment.

As the medical information data are transmitted to the inventory management division, the processing determination unit 1150 determines whether any additional entry has been made onto the implementation record card 1320 or not (S1128). If no additional entry is made onto the implementation record card 1320 (N of S1128), the processing determination unit 1150 will not proceed to a processing for transmitting the medical information data to the medical accounting division, until an additional entry is made onto the implementation record card 1320. When an additional entry is made (Y of S1128), the entry-maker classification extraction unit 1136 extracts a pen ID from the acquired medical information data and extracts the entry-maker classification information from the acquired medical information data by using the extracted pen ID and the entry-maker classification table 1116 stored in the storage unit 1110 (S1129).

As the entry-maker classification information is extracted, the processing determination unit 1150 decides, by using the extracted entry-maker classification information, whether the person who had entered the medical information data was a staff member of a medical accounting division or not (S1130). If it is determined that the person who had manually entered the medical information data was not a staff member of the medical accounting division (N of S1130), the processing determination unit 1150 will not proceed to a processing for transmitting to the medical accounting division, until an additional entry is made again. If it is determined that the person who had manually entered the medical information data was a staff member of the medical accounting division (Y of S1130), the entry position extraction unit 1138 will extract entry position information from the acquired medical information data (S1131). The partial area extraction unit 1140 extracts the partial area information indicative of a partial area in which the medical information data have been manually inputted, using the extracted entry position information. The processing determination unit 1150 identifies medical information data entered in an entry space represented by the extracted partial area information (S1132). For example, assume that in a plurality of entry spaces contained in the ninth partial area 1418 of the method-of-testing space 1322 a plurality of items of laboratory techniques are entered by a laboratory doctor or nurse and a check, a circle or the like is placed, on any of the plurality of entered items of laboratory techniques, by a staff member of the medical accounting division. In this case, the processing determination unit 1150 identifies the entry spaces where the marks are placed by the staff member of the medical accounting division, utilizing the extracted partial area information. Then the medical information data representing the items of laboratory techniques entered manually in these entry spaces are identified. As a result, the medical information data representing the implementation items, such as items of laboratory techniques, where the marks are placed by the staff members of the medical accounting division can be identified.

As the medical information data entered in the entry space represented by the extracted partial area information is identified, the processing determination unit 1150 refers to the data processing table 1118 and determines a processing for transmitting the identified medical information data to the medical accounting division (S1133). As the processing for transmitting the identified medical information data to the medical accounting division is determined, the transmission processing unit 1162 transmits the identified medical information data to the medical accounting division (S1134). In such a case, when it is determined by the processing determination unit 1150 that the medical information data be transmitted to the medical accounting division, the transmission processing unit 1162 transmits the medical information data to the workstation 1200 in the medical accounting division no matter whether a transmission request was made from the medical accounting division or not. Thereby, the workstation 1200 in the medical accounting division can receive the medical information data without a trouble of requesting the transmission thereof.

When it is determined by the processing determination unit 1150 that the medical information data be transmitted to the medical accounting division, the transmission processing unit 1162 can also transmit the medical information data to the workstation 1200 in the medical accounting division in response to a request from a data transmit request unit 1214. Thereby, the workstation 1200 in the medical accounting division can receive the medical information whenever it needs to receive the data.

The workstation 1200 in the medical accounting division receives, from the server 1100, the medical information data representing implementation items marked by the staff of the medical accounting division. The workstation 1200 performs accounting processing, utilizing the medical information data, representing the received implementation items, and the account processing information 1212. By employing the structure and the method according to the thirteenth embodiment, the items on which the accounting processing is to be performed can be easily identified by utilizing the medical information data effectively.

### Fourteenth Embodiment

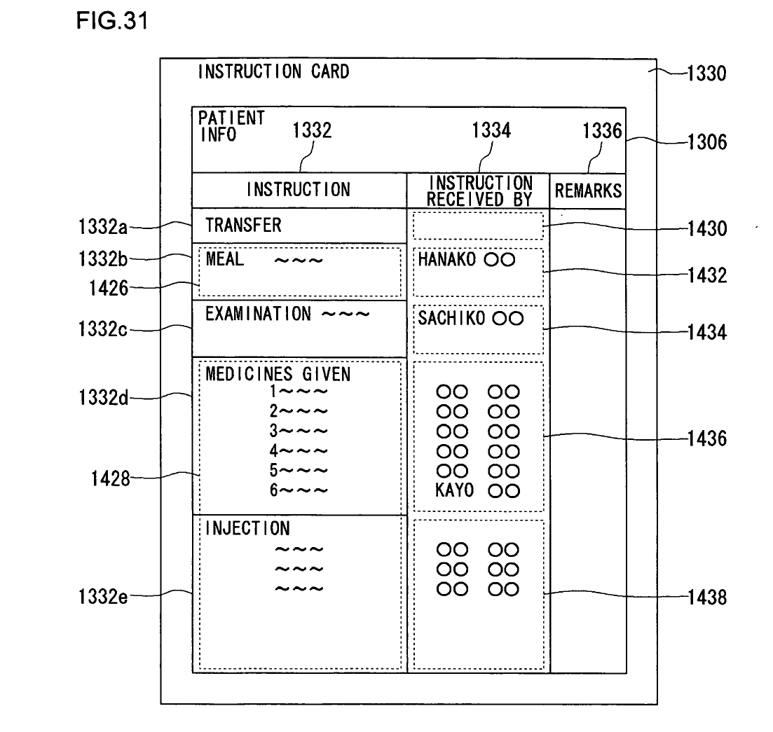

FIG. 31 illustrates an instruction card 1330 used in an electronic patient's chart system 1300 according to a fourteenth embodiment. The instruction card 1330 is prepared in such a manner that the form of an instruction card 1330 is printed on a form 1010 already printed with a dot pattern which is readable by an electronic pen 1012. The instruction card 1330 has a patient information space 1306 and is composed of an instruction space 1332, an instruction-received-by space 1334 and a remarks space 1336 which are horizontally arranged. The instruction space 1332 is composed of a transfer space 1332a, a meal space 1332b, an examination space 1332c, a medicines space 1332d and an injection space 1332e which are vertically arranged. The meal space 1332b has a thirteenth partial area 1426 as a partial area entered in the meal space 1332. The medicines space 1332d and the injection space 1332e constitute a fourteenth partial area 1428.

The instruction-received-by space 1334 contains a fifteenth partial area 1430 as a partial area where an input is entered correspondingly to the transfer space 1332a. The instruction-received-by space 1334 contains also a seventeenth partial area 1434 as a partial area where an input is entered correspondingly to the meal space 1332b. The instruction-received-by space 1334 contains also an eighteenth partial area 1436 as a partial area where an input is entered correspondingly to the medicines space 1332d. The instruction-received-by space 1334 contains also a nineteenth partial area 1438 as a partial area where an input is entered correspondingly to the injection space 1332e. The instruction-received-by space 1334 contains also a fourth implementation item space 1346 as a partial area where an input is entered correspondingly to the fourteenth partial area 1428. The instruction-received-by space 1334 contains also a nineteenth partial area 1438 as a partial area where an input is entered correspondingly to the method-of-testing space 1322.

In such the instruction card 1330, an instruction content is entered, by a physician, in the instruction space 1332. Upon receipt of an instruction from a physician, a nurse enters his/her name in the instruction space 1334 corresponding to the instruction content that he/she has received. However, when a nurse makes an entry of his/her reception corresponding to an instruction onto an instruction card 1330, the physician cannot verify whether the instruction content is presently received or not, unless the instruction card 1330 has been delivered to the physician after the nurses' reception corresponding to all the instruction contents entered onto the form of an instruction card 1330 have been completed. Hence, in the light of the case like this, a new technology is in demand where the physician can easily verify if the instruction contents instructed by the physician are received as such by the nurses.

Thus, when the instruction contents instructed by the physician are received as such by the nurses, the electronic patient's chart system 1300 according to the fourteenth embodiment, the status of medical information data representing a received instruction content is registered as an instruction-received state.

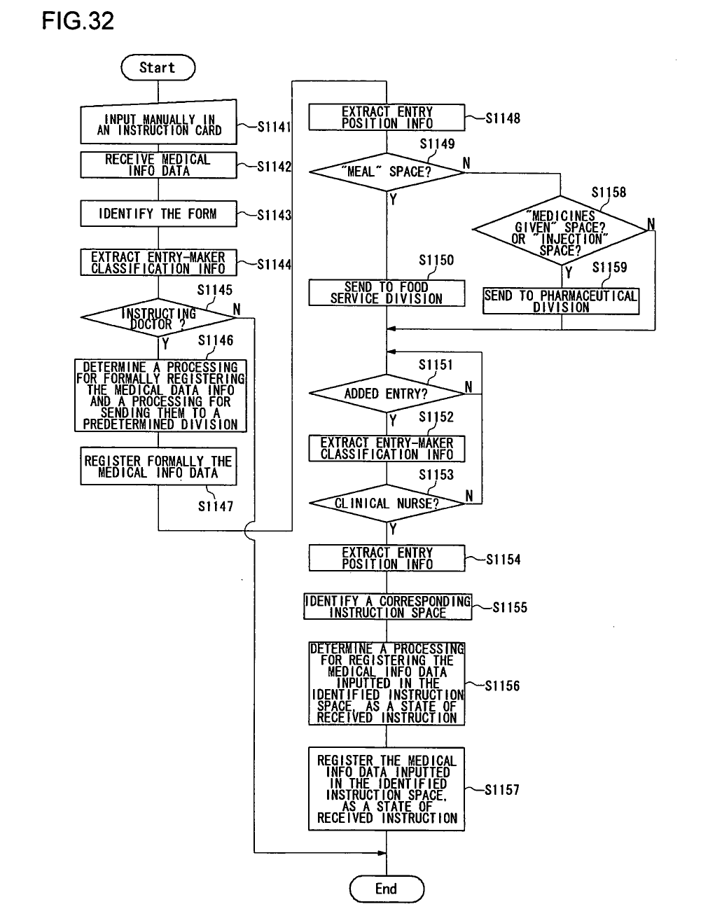

FIG. 32 is a flowchart showing the processing of an electronic patient's chart system 1300 according to the fourteenth embodiment. An instructing doctor first enters instruction contents onto an instruction card, using an electronic pen 1012, so as to input medical information data in handwriting (S1141). As the medical information data are manually inputted onto the instruction card 1330 with an electronic pen 1012, the electronic pen 1012 acquires the medical information data inputted manually onto the instruction card 1330 and transmits the acquired medical information data to a workstation 1200 in the division concerned. The workstation 1200 receives the medical information data transmitted from the electronic pen 1012. The workstation 1200 transmits the medical information data received from the electronic pen 1012 to the server 1100. The server 1100 receives the medical information data from the workstation 1200 via the network 1050 (S1142). In this manner, the server 1100 acquires the medical information data which were inputted in handwriting onto the instruction form 1330 by the instructing doctor.

As the server 1100 acquires the medical information data, a form identification unit 1132 extracts a form ID from the acquired medical information data and identifies a form 1010 entered with medical information represented by the medical information data by using the extracted form ID and a form table 1112 stored in a storage unit 1110 (S1143). As a result, a specific patient's instruction card 1330 in which the medical information data have been manually inputted is identified, and form information corresponding to this instruction card 1330 out of form information stored in the storage unit 1110 is identified. Also, the form identification unit 1132 identifies a form corresponding to this form information by identifying the form information. The received medical information data are associated with the identified form information and stored in the storage unit 1110.

As the form information is identified, an entry-maker classification extraction unit 1136 extracts a pen ID from the acquired medical information data (S1224), and extracts entry-maker classification information from the acquired medical information data, by using the extracted pen ID and an entry-maker classification table 1116 stored in a storage unit 1110 (S1144). As the entry-maker classification information is extracted, a processing determination unit 1150 decides, by using the extracted entry-maker classification information, whether the person who had entered the medical information data in handwriting was an instructing doctor or not (S1145). If it is determined that the person who had manually entered the medical information data was not an instructing doctor (N of S1145), the subsequent processings will be skipped. And if an entry has been made anew to the instruction card 1330, the processing in this flowchart will be started from S1141.

If it is determined that the person who had manually entered the medical information data was an instructing doctor (Y of S1145), the processing determination unit 1150 will refer to the data processing table 1118 and determine a processing for formally registering the medical information data and a processing for transmitting to a predetermined division (S1146). As the processing for formally registering the medical information data and the processing for transmitting to a predetermined division are determined, the registration processing unit 1164 registers formally the medical information data (S1147). The registration processing unit 1164 registers formally the medical information data in a manner that formal registration identification data indicative of being formally registered are added to the medical information data and then stored in the storage unit 1110.

As the medical information data are formally registered, the entry position extraction unit 1138 extracts entry position information from the acquired medical information data (S1148). The partial area extraction unit 1140 extracts partial area information indicative of a partial area in which the medical information data have been manually inputted, by use of the extracted entry position information. By determining whether or not the extracted partial area information corresponds to the thirteenth partial area 1426 as a partial area entered onto the meal space 1332b, the processing determination unit 1150 determines whether the instruction content is one entered in the meal space 1332b or not (S1149). If it is determined that the instruction content is one entered in the meal space 1332b (Y of S1149), the transmission processing unit 1162 transmits the instruction content to the meal division (S1150). Thus, the instruction content related to the meal can be quickly conveyed to the meal division.

If it is determined that the instruction content is not the one entered in the meal space 1332b (N of S1149), the processing determination unit 1150 will determine whether the instruction content is one entered in the medicines space 1332d or the injection space 1332e (S1158). By determining whether the acquired medical information data have been manually inputted in the fourteenth partial area 1428, the processing determination unit 1150 determines whether the instruction content is one entered in the medicines space 1332d or the injection space 1332e. If it is determined that the instruction content is one entered in the medicines space 1332d or the injection space 1332e (Y of S1158), the transmission processing unit 1162 will transmit the instruction content to the pharmaceutical division (S1159). Thus, the instruction content related to the medicines and the injection can be quickly conveyed to the pharmaceutical division. If it is determined that the instruction content is not the one entered in the medicines space 1332d or the injection space 1332e (N of S1158), the processing determination unit 1150 will determine that the necessity to convey the instruction content promptly is small and therefore skip the processing for transmitting the medical information data.

Next, the processing determination unit 1150 determines whether any additional entry has been made onto the instruction card 1330 or not (S1151). If no additional entry has been entered onto the instruction card 1330 (N of S1151), the step will not proceed to a processing of registration as an instruction-received state until an additional entry is made again. If an additional entry has been made in the instruction space 1330 (Y of S1151), the entry-maker classification extraction unit 1136 extracts a pen ID from the acquired medical information data and extracts the entry-maker classification information from the acquired medical information data by using the extracted pen ID and the entry-maker classification table 1116 stored in the storage unit 1110 (S1152). As the entry-maker classification information is extracted, the processing determination unit 1150 determines, by using the extracted entry-maker classification information, whether the person who had manually entered the acquired medical data is a nurse or not (S1153). If it is determined that the person who had manually entered the acquired medical information data was not a nurse (N of S1153), the step will not proceed to a processing of registration as an instruction-received state until an additional entry is made again.

If it is determined that the person who had manually entered the acquired medical information data is a nurse (Y of S1153), the entry position extraction unit 1138 extracts entry position information from the acquired medical information data (S1154). The partial area extraction unit 1140 extracts partial area information where the acquired medical information data have been inputted in handwriting, by use of the extracted entry position information. The processing determination unit 1150 determines which of partial areas among the fifteenth partial area 1430, the sixteenth partial area 1432, the seventeenth partial area 1434, the eighteenth partial area 1436 and the nineteenth partial area 1438 belong to the extracted partial area information. In this manner, the processing determination unit 1150 can determine which instruction space 1332 among the transfer space 1332a, the meal space 1332b, the examination space 1332c, the medicines space 1332d and the injection space 1332e the medical information data be inputted manually in, so that a specific instruction space 1332 corresponding to the acquired medical information data can be identified. As described above, the processing determination unit 1150 identifies a corresponding instruction space 1332 (S1155).

As the corresponding instruction space 1332 is identified, the processing determination unit 1150 refers to the data processing table 1118 and determines a processing for registering the medical information data entered in the identified instruction space as an instruction-received state (S1156). As the processing for registering the medical information data entered in the identified instruction space as an instruction-received state is determined, the registration processing unit 1164 registers the medical information data entered in the identified instruction space, as an instruction-received state (S1157). The registration processing unit 1164 registers the medical information data entered in the identified instruction space as an instruction-received state, in a manner that instruction-received-state identification data representing that the medical information data are in an instruction-received state is appended to the medical information data.

It is to be noted that if a nurse enters a statement indicating that an instruction has been received, onto the instruction card 1330, only a tick mark or the like may be entered onto the instruction-received-by space 1334. The entry-maker extraction unit 1134 extracts entry-maker information from the medical information data representing an entry content such as a tick mark or the like entered in the instruction-received-by space 1334. If a user such as a physician browses the form information corresponding to this instruction card 1330, the output processing unit 1166 of the server 1100 or the output processing unit 1218 of the workstation 1200 use the extracted entry-maker information, display on a display the names of entry-makers at positions where the checks are entered and print them by a printer. This also enables physicians to verify the names of nurses who have received the instructions. By employing the structure and the method as described above, the physicians can easily verify whether the instruction contents instructed by the physicians are duly received by nurses or not.

### Fifteenth Embodiment

In the instruction card 1330 shown in FIG. 31, after the instruction contents written by a physician onto the instruction card 1332 have been instruction-received by nurses, they may be added with some additional entries or may be deleted in part or whole. In such a case, when the instruction card 1330 where the instruction contents thereof are added with some additional entries or they are deleted in part or whole is used as it is, excessive amount of description may be written onto the instruction card 1330. This may cause difficulty in judging at first sight the state of instruction for the instruction contents entered. For that reason, an electronic patient's chart system 1300 according to a fifteenth embodiment utilizes the acquired medical information data effectively in a manner that if the instruction contents of the instruction card 1330 are added with additional entries or deleted in part or whole and the instruction card 1330 is printed again, the added entries and/or the deletion are effected on the instruction card 1330 to be printed again.

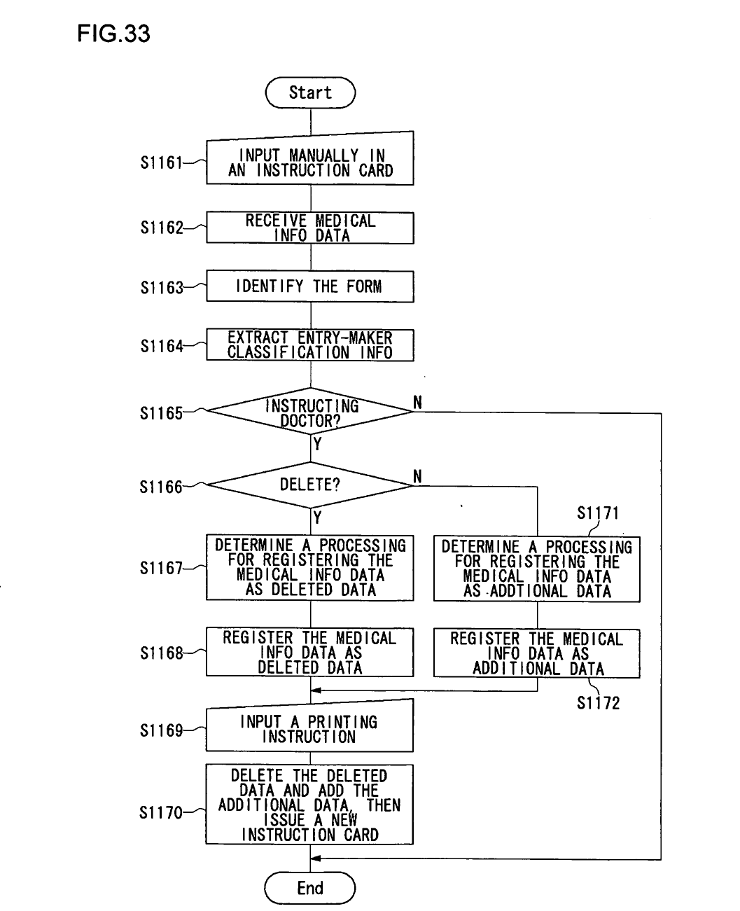

FIG. 33 is a flowchart showing the processing of an electronic patient's system 1300 according to the fifteenth embodiment.

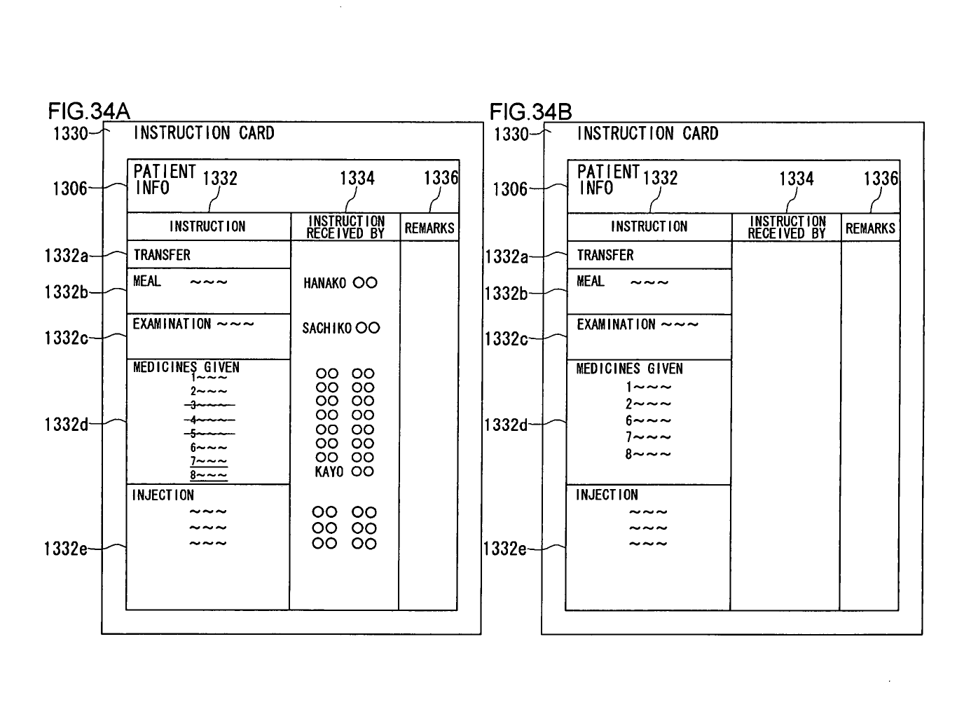

An instructing doctor makes an entry of medical information data, indicating the addition or deletion of an instruction content, onto an instruction card 1330 where the instruction contents have already been entered, by use of an electronic pen 1012 (S1161). The addition of an instruction content means that the instruction content is added to the current instruction contents in a manner that the added instruction content does not overlap with the entries of the current instruction contents, as shown in FIG. FIG. 34A. In the example of FIG. 34A, the instruction contents 1 to 6 were entered in the medicines space 1332d but after the addition, added contents 7 and 8 are now entered below the instruction contents 1 to 6 in the instruction space 1332. The deletion of an instruction content means that marks or the like for deleting the present instruction content in part or whole are entered onto the present instruction content in an overlapped manner by use of strikethroughs, for example. In the example of FIG. 34B, the instruction contents 1 to 6 were entered in the medicines space 1332d but after the deletion, the strikethroughs are entered onto the instruction contents 3 to 5 in an overlapped manner. Note that the strikethrough or a line-through shown in FIG. 34A may be double lines, or markings such as x may be used.

As the medical information data are entered in handwriting onto the instruction card 1330, a workstation 1200 in the division concerned receives the medical information data acquired by the electronic pen 1012. A server 1100 receives from the workstation 1200 the medical information data acquired by the electronic pen 1012, via a network 1050 (S1162).

As the server 1100 acquires the medical information data, a form identification unit 1132 extracts a form ID from the acquired medical information data and identifies a form 1010 entered with medical information represented by the medical information data by using the extracted form ID and a form table 1112 stored in a storage unit 1110 (S1163). As a result, a specific patient's instruction card 1330 in which the medical information data have been manually inputted is identified, and form information corresponding to this instruction card 1330 out of form information stored in the storage unit 1110 is identified. Also, the form identification unit 1132 identifies a form corresponding to this form information by identifying the form information. The received medical information data are associated with the identified form information and stored in the storage unit 1110.

As the form information is identified, an entry-maker classification extraction unit 1136 extracts a pen ID from the acquired medical information data, and extracts entry-maker classification information from the acquired medical information data, by using the extracted pen ID and an entry-maker classification table 1116 stored in the storage unit 1110 (S1164). As the entry-maker classification information is extracted, a processing determination unit 1150 decides, by using the extracted entry-maker classification information, whether the person who had entered the medical information data in handwriting was an instructing doctor or not (S1165).

If it is determined that the person who had manually entered the medical information data was not an instructing doctor (N of S1165), the processing determination unit 1150 will determine, based on the acquired medical information data, that there is no need to register the medical information data as added data or deleted data, and then terminate the processing in this flowchart.

If it is determined that the person who had manually entered the medical information data was an instructing doctor (Y of S1165), the processing determination unit 1150 will determine whether medical information represented by the acquired medical information data is a mark for deleting the content currently entered onto the instruction card 1330 or not (S1166). The processing determination unit 1150 determines whether or not the medical information represented by the acquired medical information data contains a line having a predetermined length or more and this line is written onto the content currently entered onto the instruction card 1330 in an overlapped manner. This determines whether the medical information indicated by the acquired medical information data is the mark for deleting the content currently entered onto the instruction card 1330 or not.

If it is determined that the medical information represented by the acquired medical information data was the mark for deleting the content currently entered onto the instruction card 1330 (Y of S1166), the processing determination unit 1150 will refer to the data processing table 1118 and determine a processing for registering the medical information data, representing the content in which the mark for deletion is entered, as deletion data (S1167). As the processing performed on the medical information data is determined, the registration processing unit 1164 registers as deletion data the medical information data representing the mark for deletion and the medical information data, representing the content in which the mark for deletion is entered in an overlapped manner (S1168). By appending deletion identification data indicative of deletion data to the medical information data and then storing them in the storage unit 1110, the processing determination unit 1150 registers as deletion data the medical information data representing the mark for deletion and the medical information data, representing the content in which the mark for deletion is entered in an overlapped manner.

If it is determined that the medical information represented by the acquired medical information data was not a mark for deleting the content currently entered onto the instruction card 1330 (N of S1166), the processing determination unit 1150 will refer to the data processing table 1118, determine that the manually inputted medical information data are the added data and then determine a processing for registering the acquired medical information data as added data (S1171). As the processing for registering the medical information data as added data is determined, the output processing unit 1166 registers the medical information data as added data (S1172). The processing determination unit 1150 registers the medical information data as added data, by appending addition identification data indicative of added data to the medical information data and then storing them in the storage unit 1110. Note that the medical information data may be registered as added data, by not appending the deletion indication data thereto and storing the medical information data intact.

In this manner, if a user enters a print instruction input for printing the instruction card 1330 again after the medical information data have been registered as deletion data or added data (S1169), the output processing unit 1166 will perform a print output processing in response to the registration status of the medical information data (S1170). More specifically, the output processing unit 1166 removes the medical information data registered as deletion data, from the medical information data to be outputted by printing. Thus, when the instruction card 1330 is printed again, the medical information data registered as deletion data are not printed. In the example of FIG. 34B, the instruction contents 1003 to 1005 with the strikethroughs entered, in the instruction contents entered in the medicines space 1332d, are not printed.

On the other hand, the medical information data registered as added data are included in the medical information data to be outputted by printing. Thus, when the instruction card 1330 is printed again, the medical information data registered as added data are printed. In the example of FIG. 34B, the instruction contents 3 to 5 with the strikethroughs entered, in the instruction contents entered in the medicines space 1332d, are not printed. In such a case, some space may be left open in the medical information such as instruction contents printed on the instruction card 1330 because the medical information data registered as deletion data were not printed there. In the light of this, if some space is left open in the medical information printed on the instruction card 1330, the output processing unit 1166 will move the printing position, at which the medical information is printed, so that this unused space is narrowed. In the example of FIG. 34B, the instruction contents 7 and 8 added in the medicines space 1332d are printed. In this case, the instruction contents 6 to 8 are moved and printed so that the spaces created by the deletion of the instruction contents 3 to 5 are narrowed.

The electronic patient's chart system 1300 may register the only medical information data manually entered onto the instruction space 1332, as the added or deleted data. In such a case, the partial area extraction unit 1140 extracts partial area information of the acquired medical information data. The processing determination unit 1150 determines, by using the extracted partial area information, whether the added content, the deletion mark or the like is the one entered onto the instruction space 1332 or not. If it is determined that the added content, the deletion mark or the like is the one entered onto the instruction space 1332, the processing determination unit 1150 will proceed to S1164 in this flowchart. As a result, only the addition or deletion entered by a physician can be reflected on the instruction card 1330 to be printed anew. In this manner, even in a case where there is added entries or deletion to the instruction card 1330, the instruction contents entered onto the instruction card 1330 can be made easy-to-understand when the instruction card 1330 is printed.

### Sixteenth Embodiment

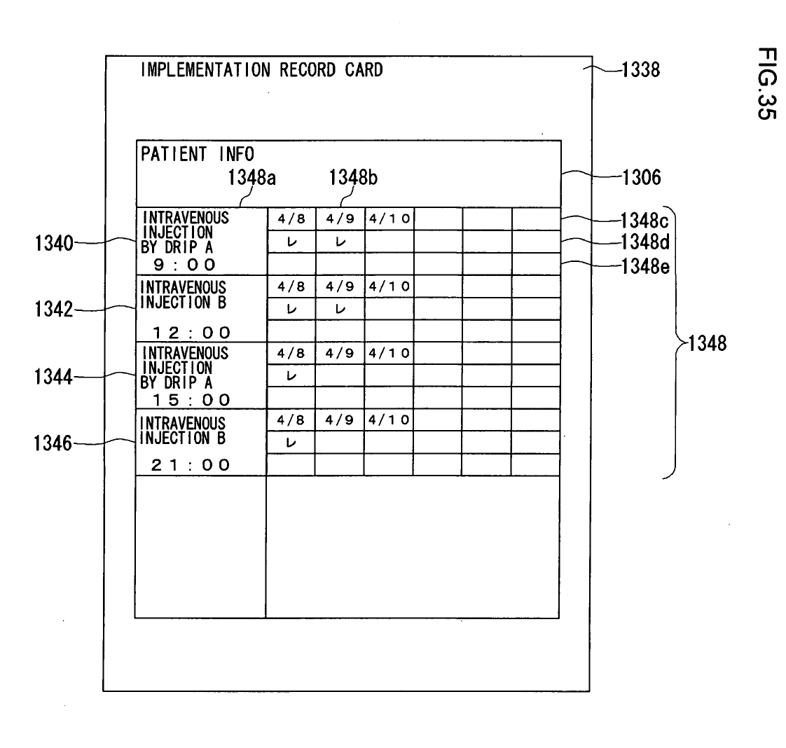

FIG. 35 illustrates an implementation record card 1338 used in an electronic patient's chart system 1300 according to a sixteenth embodiment. The implementation record card 1338 is prepared in such a manner that the form of an implementation record card 1338 is printed on a form 1010 already printed with a dot pattern which is readable by an electronic pen 1012. The implementation record card 1338 has a patient information space 1306 and an implementation item space 1348. The implementation item space 1348 has a first implementation item space 1340, a second implementation item space 1342, a third implementation item space 1344 and a fourth implementation item space 1346. Each of the first implementation item space 1340 to the fourth implementation item space 1346 has an item name space 1348a and a schedule space 1348b. Further, the schedule space 1328b is composed of a date space 1348c, a check space 1348d and a reserved space 1348e.

In such an implementation record card 1338, a new technology is in demand that verifies the implementation status of each implementation item by effectively utilizing the medical information data. For that purpose, when the implementation status of each implementation item is entered, the electronic patient's chart system 1300 registers an entry-maker as a person who implements the item entered, or an action-taker, and registers the entry time as the time when an implementation takes place.

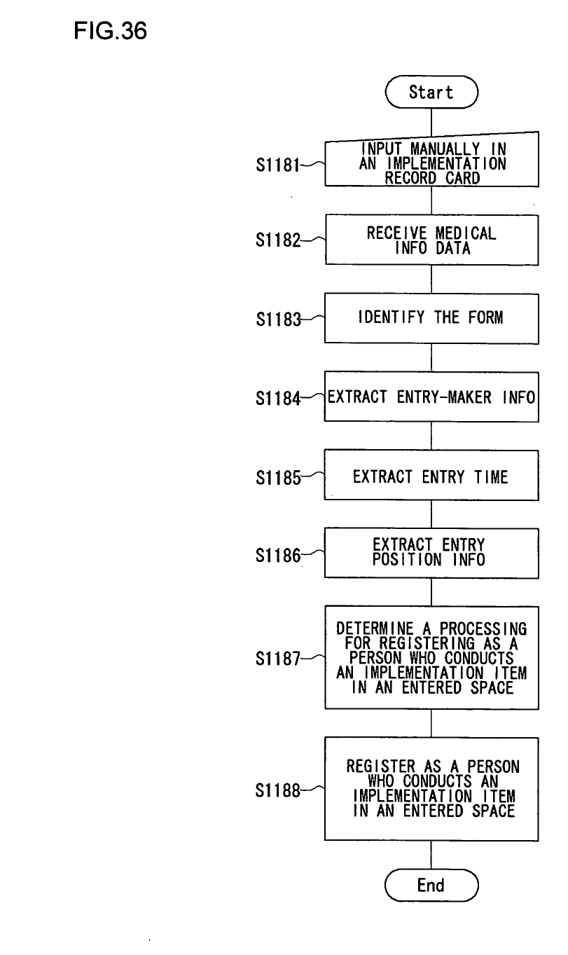

FIG. 36 is a flowchart showing the processing of an electronic patient's system 1300 according to the sixteenth embodiment. In the item name space 1348a of the implementation record card 1338, item names of implementation items and the time at which an action corresponding to the item is to be taken are entered beforehand by a physician. Following a physician's instructions entered onto the implementation record card 1338, a nurse carries out the implementation items corresponding to the implementation names entered onto the item name space 1348a, at the time when the item is to be attended. When carrying out the implementation items entered onto the item name space 1348a is completed, the nurse makes an entry of medical information data including the dates, tick marks and the like in the schedule space 1348b of the implementation item space 1348 in handwriting (S1181).

As the medical information data are entered in handwriting onto an instruction card by use of an electronic pen 1012, a workstation 1200 in the division concerned receives the medical information data acquired by the electronic pen 1012. A server 1100 receives from the workstation 1200 the medical information data acquired by the electronic pen 1012, via a network 1050 (S1182). As the server 1100 acquires the medical information data, a form identification unit 1132 extracts a form ID from the acquired medical information data and identifies a form 1010 entered with medical information represented by the medical information data by using the extracted form ID and a form table 1112 stored in a storage unit 1110 (S1183). As a result, a specific patient's implementation record card 1338 in which the medical information data have been manually inputted is identified, and form information corresponding to this implementation record card 1338 out of form information stored in the storage unit 1110 is identified. Also, the form identification unit 1132 identifies a form corresponding to this form information by identifying the form information. The received medical information data are associated with the identified form information and stored in the storage unit 1110.

As the form information is identified, an entry-maker extraction unit 1134 extracts entry-maker information from the acquired medical information data (S1184). As the entry-maker information is extracted, an entry time extraction unit 1142 extracts entry time information from the acquired medical information data (S1185). The entry time extraction unit 1142 extracts the entry time information attached to the medical information data by the time information assignment part 1028 of the electronic pen 1012 so as to extract the entry time information from the acquired medical information data. As the entry time information is extracted, the entry position extraction unit 1138 extracts the entry position information from the acquired medical information data (S1186). A partial area extraction unit 1140 extracts partial area information where the acquired medical information data have been manually entered, using the extracted entry position information. The processing determination unit 1150 identifies an item name space 1348a corresponding to the medical information entered, using the extracted partial area information.

Next, the processing determination unit 1150 refers to the data processing table 1118 and determines the execution of a processing for registering action-takers and a processing for registering the implementation time, to the medical information data representing the item names entered in the item name space 1348a corresponding to the spaces with the tick marks entered (S1187). As the processing performed on the medical information data is determined, the registration processing unit 1164 registers the action-takers and the implementation time for the medical information data representing the item names entered in the item name space 1348a corresponding to the spaces with the tick marks entered (S1188).

In this case, using the extracted partial area information, the registration processing unit 1164 identifies which implementation item space among the first implementation item space 1340 to the fourth implementation item space 1346 the medical information data are manually entered to. Next, the registration processing unit 1164 identifies the medical information data which have been manually entered in the item name space 1348a in the extracted implementation item space. The registration processing unit 1164 associates the extracted medical information data with the entry-maker information and the entry time information extracted from the medical information data acquired by manually inputting tick marks, and then stores them. Thereby, the registration processing unit 1164 registers the action-takers and the implementation time for the medical information data representing the item names entered in the item name space 1348a.

When the user browses the form information corresponding to this implementation record card 1338, the output processing unit 1166 of the server 1100 or the output processing unit 1218 of the workstation 1200 associates each implementation item entered in the item name space 1348a in the implementation record card 1338 with the action-taker and the implementation time for this implementation item and then displays them on a display, using the entry-maker information and the entry time information stored in association with the medical information data. As a result, the implementation status of each implementation item can be easily verified externally by, for example, browsing the form information corresponding to the implementation record card 1338.

### Seventeenth Embodiment

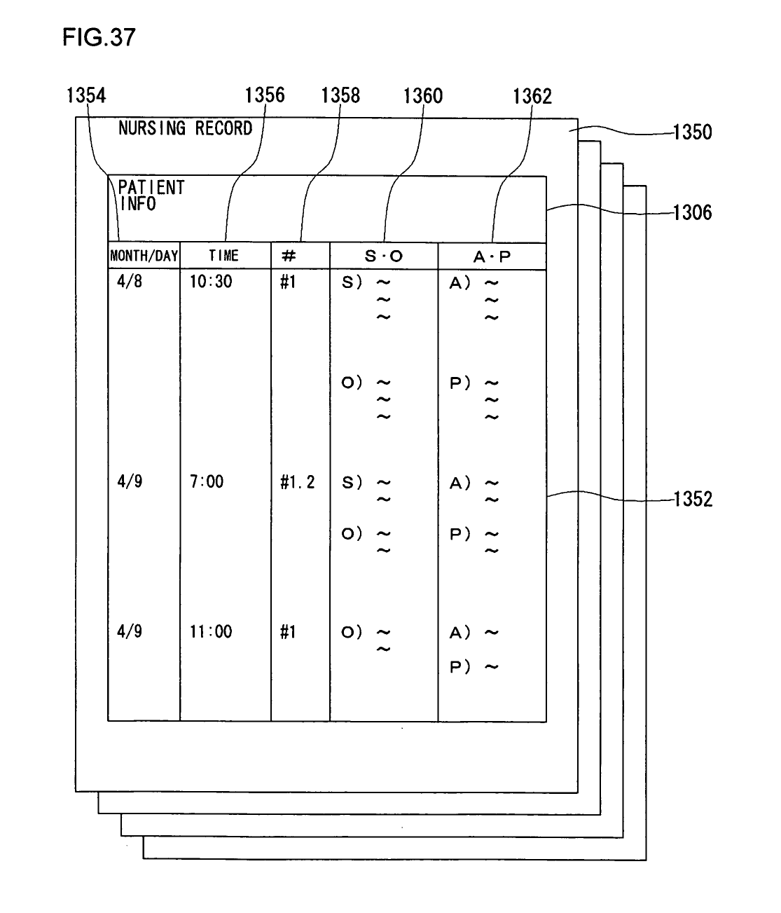

FIG. 37 illustrates a nursing record form 1350 used in an electronic patient's chart system 1300 according to a seventeenth embodiment. The nursing record form 1350 is prepared in such a manner that the form of a nursing record form 1350 is printed on a form 1010 already printed with a dot pattern which is readable by an electronic pen 1012. The nursing record form 1350 has a patient information space 1306 and a nursing record space 1352. The nursing record space 1352 is composed of a date space 1354, a time column 1356, a problem number space 1358, an S-O space 1360, and an A-P space 1362 which are horizontally arranged. The problem numbers are entered in the problem number space 1358. The problem numbers meant here are numbers representing the problems related to the patients and, for example, “#1” represents “numbness in his/her hand is reported” and “#2” represents “emotional distress” and the like. Subjective information and objective information are entered in the S-O space 1360. Assessment and planning are entered in the A-P space 1362.

When the nurses make entries onto the nursing forms 1350 like this, there are cases where different attending nurses make entries onto the same nursing record form 135, for instance, and it is difficult to later identify the nurses who made entries onto the nursing record form 1350. The nursing record form 1350 used in the electronic patient's chart system according to the seventeenth embodiment 1300 has the date space 1354 and the time column 1356 but there are cases, for example, where the date and time entered in the problem number space 1358, S-o space 1360 or A-P space 1362 differ from the data and time recorded in the date space 1354 and the time column 1356. In such a case, it may be difficult to later identify the time of entry made onto the nursing record form 1350. Hence, a new technology is in demand that can accurately grasp the history of entry to the nursing record form 1350 by making an effective use of the medical information data. In response to this demand, the electronic patient's chart system 1300 according to the seventeenth embodiment is so arranged that when an entry is made onto the nursing record form 1350, an entry-maker and entry time are registered.

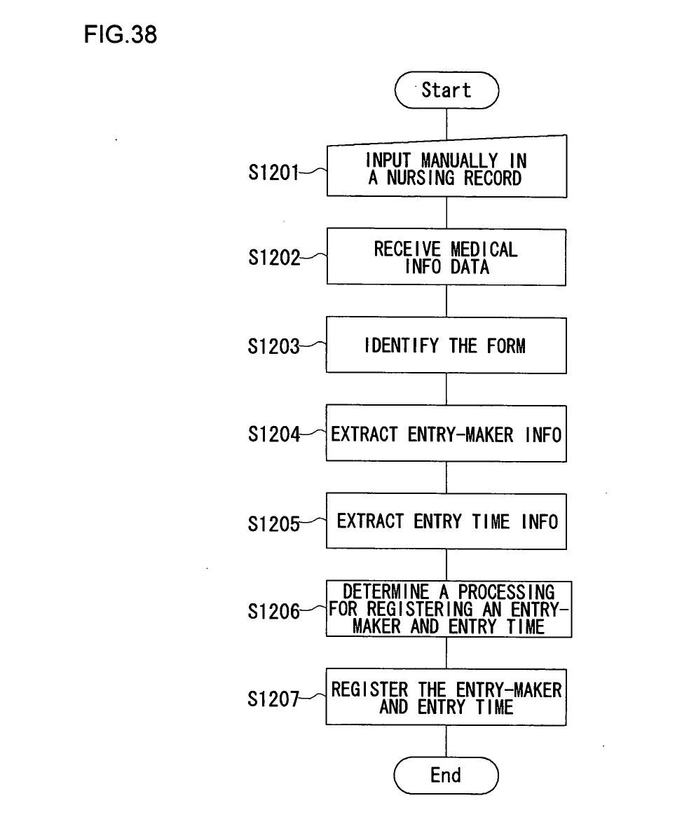

FIG. 38 is a flowchart showing the processing of an electronic patient's chart system 1300 according to the seventeenth embodiment. A nurse first manually inputs medical information data onto a nursing record form 1350 using an electronic pen 1012 (S1201). As the medical information data are manually inputted onto the nursing record form 1350 with an electronic pen 1012, a workstation 1200 in the division concerned receives the medical information data acquired by the electronic pen 1012. A server 1100 receives the medical information data acquired by the electronic pen 1012 from the workstation 1200 via a network 1050 (S1202).

As the server 1100 acquires the medical information data, a form identification unit 1132 extracts a form ID from the acquired medical information data and identifies a form 1010 entered with medical information represented by the medical information data by using the extracted form ID and a form table 1112 stored in a storage unit 1110 (S1203). As a result, a specific patient's nursing record form 1350 in which the medical information data have been manually inputted is identified, and form information corresponding to this nursing record form 1350 out of form information stored in the storage unit 1110 is identified. Also, the form identification unit 1132 identifies a form corresponding to this form information by identifying the form information. The received medical information data are associated with the identified form information and stored in the storage unit 1110.

As the form information is identified, the entry-maker extraction unit 1134 extracts entry-maker information (S1204). As the entry-maker is extracted, the entry time extraction unit 1142 extracts entry time information from the acquired medical information data (S1205)

As the entry-maker information and the entry time information are extracted, the processing determination unit 1150 refers to a data processing table and decides on a processing for registering an entry-maker who has made the entry onto the nursing record form and a processing for registering the entry time (S1206). As the processing has been decided on, a registration processing unit 1164 registers the entry-maker who has made the entry onto the nursing record form 1305 and the entry time (S1207). The registration processing unit 1164 associates the acquired medical information data with the extracted entry-maker information and then stores them in the storage unit 1110 so as to register the entry-maker who has made the entry onto the nursing record form 1350.

It is to be noted that in this case the entry maker who has made an entry in the date space 1354, the time column 1356 or the problem number space 1358 may be registered as a recognizing entry-maker and the entry maker who has made an entry in the S-O space 1360 or A-P space 1362 may be registered as an evaluator. In such a case, the partial area extraction unit 1140 extracts partial area information from the acquired medical information data. The registration processing unit 1164 determines which space the medical information data have been manually inputted, by using the extracted partial area information. When it is determined that the acquired medical information data have been manually inputted to the date space 1354, the time column 1356 or the problem number space 1358, the registration processing unit 1164 adds recognizing entry-maker identification data, indicative of a recognizing entry-maker, to the extracted entry-maker information and then stores them in the storage unit 1110. When it is determined that the acquired medical information data have been manually inputted to the S-O space 1360 or A-P space 1362, the registration processing unit 1164 adds evaluator identification data, indicative of an evaluator, to the extracted entry-maker information and then stores them in the storage unit 1110. Thereby, it is possible to identify whether the nurse who has made an entry onto the nursing record form 1350 is a person who recognizes the problem or a person who evaluates it. The registration processing unit 1164 associates the acquired medical information data with the entry time before storing them, so that the time of entry made onto the nursing record form 1350 are registered and saved.

In the case when the user browses the form information corresponding to this nursing record form 1350 and in this manner the user browses the medical information data in which the action-takers and the time of actions are saved, the output processing unit 1166 of the server 1100 or the output processing unit 1218 of the workstation 1200 utilizes the entry-maker information, which has been associated with the medical information data and then stored, and also associates the manually inputted medical information data with the nursing record form 1350 so as to display the entry-makers, the entry time and the like on a display. As a result, the electronic patients' charts corresponding to the nursing record form 1350 are viewed and therefore the history of entries in the nursing record form can be easily verified from outside.

### Eighteenth Embodiment

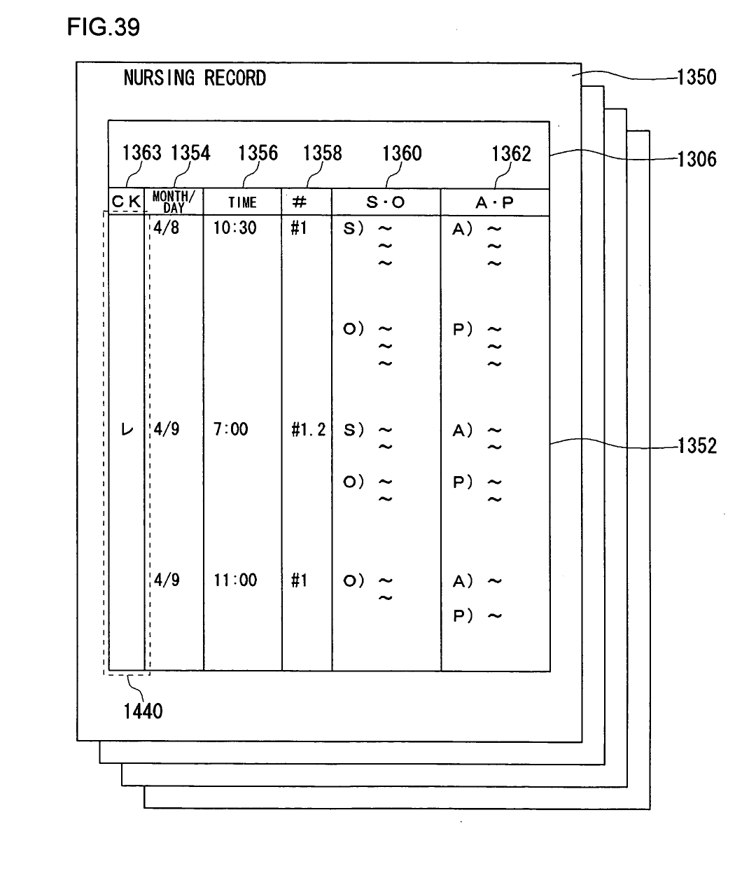

FIG. 39 illustrates a nursing record form 1350 used in an electronic patient's chart system 1300 according to an eighteenth embodiment. The nursing record form 1350 is prepared in such a manner that the form of a nursing record form 1350 is printed on a form 1010 already printed with a dot pattern which is readable by an electronic pen 1012. The nursing record form 1350 according to the eighteenth embodiment is equal to a nursing record form 1350 according to the seventeenth embodiment added with a check space 1363. The check space 1363 includes a twentieth partial area 1440 as a partial area where a check is entered. Note that the medical information to be entered in the check space 1363 is not limited to a check mark, but any other code specifying nursing record may be entered there.

There are cases where the contents of finished entry in the nursing record form 1350 are made into a nursing summary. In such a case, part of the medical information entered in the nursing record form 1350 may be transcribed to a nursing summary. To prepare such a nursing summary, a nurse has to enter the medical information once entered in the nursing record form 1350 again in the nursing summary. Hence, a new technology is in demand that enables an easy transcription of medical information entered in the nursing record form 1350 to a nursing summary by making an effective use of the medical information data. In response to this demand, the electronic patient's chart system 1300 according to the eighteenth embodiment is so arranged that when a check is placed in the check space 1363, the nursing record corresponding to this check is saved in nursing summary data.

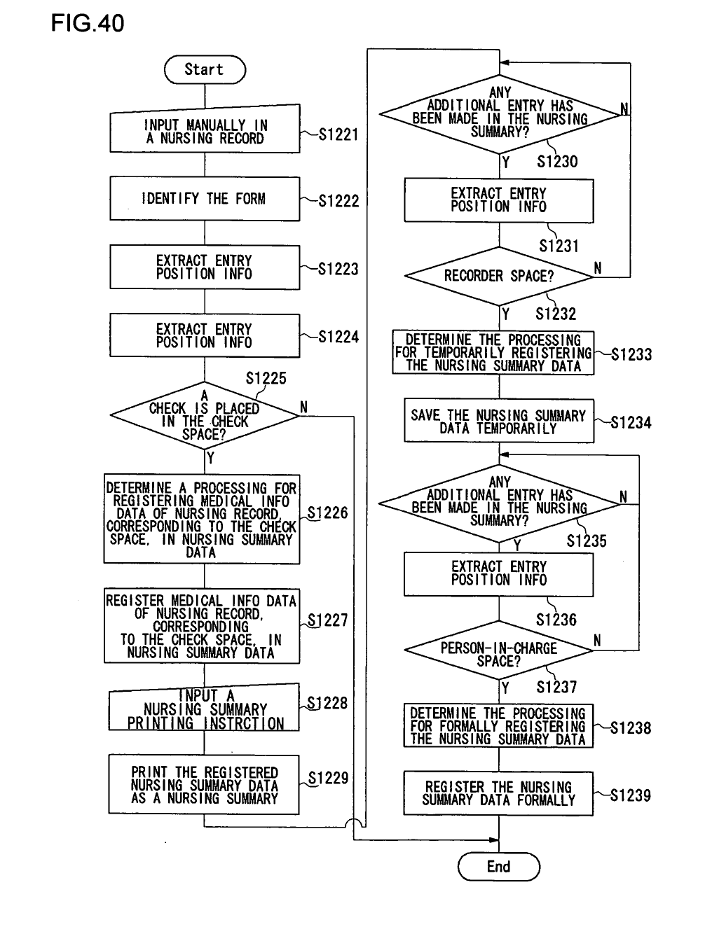

FIG. 40 is a flowchart showing the processing of an electronic patient's chart system 1300 according to the eighteenth embodiment. A nurse manually inputs medical information data, representing a check mark, in the check space 1363 of a nursing record form 1350 using an electronic pen 1012 (S1221).

As the medical information data are manually inputted onto the nursing record form 1350 with an electronic pen 1012, a workstation 1200 in the division concerned receives the medical information data acquired by the electronic pen 1012. A server 1100 receives the medical information data acquired by the electronic pen 1012 from the workstation 1200 via a network 1050 (S1222).

As the server 1100 acquires the medical information data, a form identification unit 1132 extracts a form ID from the acquired medical information data and identifies a form 1010 entered with medical information represented by the medical information data by using the extracted form ID and a form table 1112 stored in a storage unit 1110 (S1223). As a result, a specific patient's nursing record form 1350 in which the medical information data have been manually inputted is identified, and form information corresponding to this nursing record form 1350 out of form information stored in the storage unit 1110 is identified. Also, the form identification unit 1132 identifies a form corresponding to this form information by identifying the form information. The received medical information data are associated with the identified form information and stored in the storage unit 1110.

As the form information is identified, an entry position extraction unit 1138 extracts the entry position information from the acquired medical information data (S1224). A partial area extraction unit 1140 extracts partial area information for the manual input of the acquired medical information data by using the extracted entry position information. A processing determination unit 1150 decides whether a check is placed in the check space or not, in a manner that whether the extracted partial information indicates the twentieth partial area 1440 or not is determined (S1225).

When it is decided that no check is placed in the check space (N of S1225), the processing determination unit 1150 decides that none of the nursing record entered in the nursing record form 1350 is to be transcribed to the nursing summary and thereby terminates the processing in this flowchart.

When it is decided that a check is placed in the check space (Y of S1225), the processing determination unit 1150 refers to a data processing table 1118 and decides on a processing for registering the medical information data of the nursing record, corresponding to the check, in nursing summary data (S1226). The processing determination unit 1150 identifies the position where the check is entered by using the input position of the medical information data. The processing determination unit 1150 identifies the medical information data representing the date entered in a position closest to the position where the check is entered, out of the dates entered in the date space 1354. The processing determination unit 1150 further identifies the medical information data representing the nursing record corresponding to this date. In this manner, the processing determination unit 1150 identifies the medical information data of the nursing record corresponding to the check.

As the processing for registering the medical information data of the nursing record, corresponding to the check, in nursing summary data is decided on, a registration processing unit 1164 registers the medical information data of the nursing record, corresponding to the check, in the nursing summary data (S1227). The registration processing unit 1164 associates the medical information data identified as the nursing record, corresponding to the check, with the nursing summary information representing the nursing summary to which the former is to be transcribed before storing these data. In this manner, the registration processing unit 1164 registers the medical information data of the nursing record, corresponding to the check, in the nursing summary data.

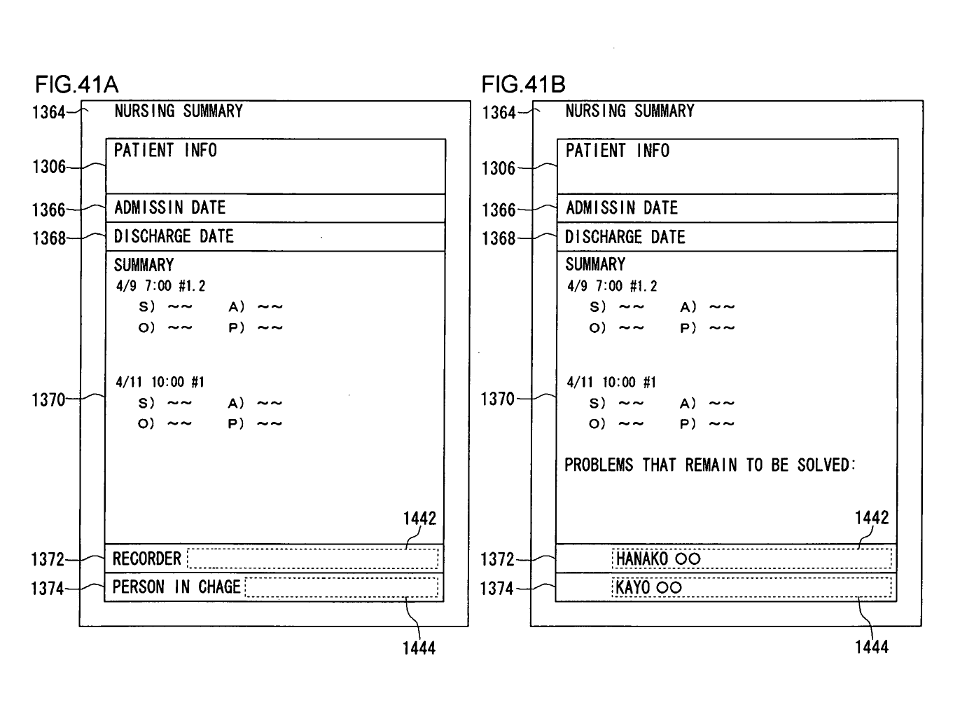

When a nursing summary printing instruction input is made by a user after the medical information data of the nursing record, corresponding to the check, is registered in the nursing summary data (S1228), an output processing unit 1166 prints the registered nursing summary data as the nursing summary (S1229). FIG. 41A illustrates a nursing summary form 1364 that is printed in an electronic patient's chart system according to the eighteenth embodiment. The nursing summary form 1364 is prepared in such a manner that the form of a nursing summary 1364 is printed on a form 1010 already printed with a dot pattern which is readable by an electronic pen 1012. The nursing summary 1364 includes a patient information space 1306, an admission date space 1366, a discharge date space 1368, a summary space 1370, a recorder space 1372, and a person-in-charge space 1374. The recorder space 1372 includes a twenty-first partial area 1442 as a partial area where the name of a recorder is to be inputted. Also, the person-in-charge space 1374 includes a twenty-second partial area 1444 as a partial area where the name of a person in charge is to be inputted. The output processing unit 1166 prints the registered nursing summary data in this summary space 1370. As a result, it is possible to easily transcribe the nursing record entered in the nursing record form 1350 to the nursing summary form 1364 by effectively using the medical information data.

When a name of a recorder is entered in the recorder space 1372 of the nursing summary form 1364 as shown in FIG. 41B, the electronic patient's chart system 1300 temporarily registers the nursing summary data of the nursing summary form 1364. And when a name of a person in charge is entered in the person-in-charge space 1374, the electronic patient's chart system 1300 formally registers the nursing summary data of the nursing summary form 1364. Therefore, if the registered nursing summary data are printed in the nursing summary form 1364, the processing determination unit 1150 will decide whether any additional entry has been made in the nursing summary form 1364 or not (S1230). When no additional entry has been made in the nursing summary form 1364 (N of S1230), there is no entry of the name of a recorder, so that the processing determination unit 1150 does not proceed to the processing of temporarily saving the nursing summary data.

When any additional entry has been made in the nursing summary form 1364 (Y of S1230), an entry position extraction unit 1138 extracts entry position information from the acquired medical information data (S1231). A partial area extraction unit 1140 extracts the partial area information indicative of a partial area in which the medical information data have been manually inputted by using the extracted entry position information. By deciding whether the extracted partial area information represents the twenty-first partial area 1442 or not, the processing determination unit 1150 decides whether any additional entry has been made in the recorder space 1372 or not (S1232). The processing determination unit 1150 decides on whether any additional entry has been made in a recorder space 1372 or not. When it is decided that no additional entry has been made in the recorder space 1372 (N of S1232), the processing determination unit 1150 does not proceed to the processing of temporarily registering the nursing summary data until an additional entry is made again.

When it is decided that any additional entry has been made in the recorder space 1372 (Y of S1232), the processing determination unit 1150 refers to the data processing table 1118, decides that the name of the recorder has been entered in the person-in-charge space 1374, and selects the processing for temporarily registering the nursing summary data (S1233). As the processing for temporarily registering the nursing summary data is selected, the registration processing unit 1164 saves the nursing summary data temporarily (S1234). The registration processing unit 1164 saves the nursing summary data temporarily by storing in the storage unit 1110 a temporary registration identification data representing a state of temporary registering in correspondence to the form information corresponding to the nursing summary form 1364.

When the nursing summary data are temporarily registered, the processing determination unit 1150 decides whether any additional entry has been made in the nursing summary form 1364 or not (S1235). When no additional entry has been made in the nursing summary form 1364 (N of S1235), there is no entry of the name of a person in charge in the person-in-charge space 1374, so that the processing determination unit 1150 does not proceed to the processing of formally registering the nursing summary data.

When any additional entry has been made in the nursing summary form 1364 (Y of S1235), the entry position extraction unit 1138 extracts entry position information from the acquired medical information data (S1236). The partial area extraction unit 1140 extracts the partial area information indicative of a partial area in which the medical information data have been manually inputted by using the extracted entry position information. By deciding whether the extracted partial area information represents the twenty-second partial area 1444 or not, the processing determination unit 1150 decides whether any additional entry has been made in the person-in-charge space 1374 or not (S1237). When it is decided that no additional entry has been made in the person-in-charge space 1374 (N of S1237), the processing determination unit 1150 does not proceed to the processing of formally registering the nursing summary data until an additional entry is made again.

When it is decided that any additional entry has been made in the person-in-charge space 1374 (Y of S1237), the processing determination unit 1150 refers to the data processing table 1118 and selects the processing for formally registering the nursing summary data (S1238). As the processing for formally registering the nursing summary data is selected, the registration processing unit 1164 registers the nursing summary data formally (S1239). The registration processing unit 1164 registers the nursing summary data formally by storing in the storage unit 1110 a formal registration identification data representing a state of formal registering in correspondence to the form information corresponding to the nursing summary form 1364.

When the nursing summary data in the nursing summary form 1364 is to be displayed on the display, the output processing unit 1166 of the server 1100 or the output processing unit 1218 of the workstation 1200 produces displays in different display modes between when the nursing summary data are temporarily registered and when it is formally registered. Different display modes meant here are such that, for example, “Temporary Registration” may be displayed in the display of nursing summary data when the nursing summary data are temporarily saved, and “Formal Registration” when the nursing summary data are formally registered. As a result, a person who browses the nursing summary data can easily see whether the nursing summary data are temporarily registered or formally registered.

### Nineteenth Embodiment

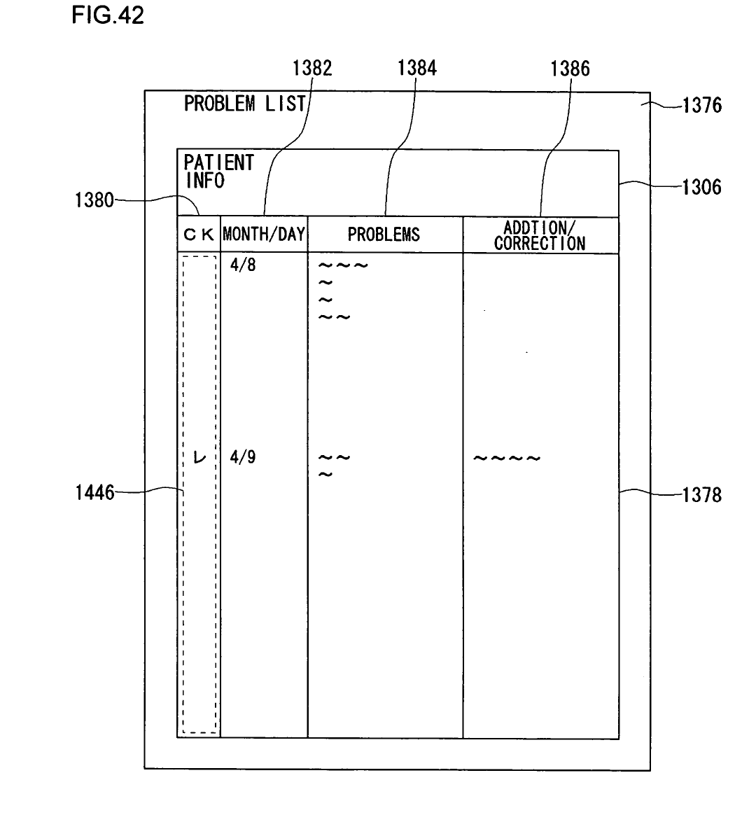

FIG. 42 illustrates a problem list form 1376 used in an electronic patient's chart system 1300 according to a nineteenth embodiment. The problem list form 1376 is prepared in such a manner that the form of a problem list form 1376 is printed on a form 1010 already printed with a dot pattern which is readable by an electronic pen 1012. The problem list form 1376 includes a patient information space 1306 and a problem list space 1378. The problem list space 1378 is composed of a check space 1380, a date space 1382, a problem space 1384, and an addition/correction space 1386, which are horizontally arranged. Provided in the check space 1380 is a twenty-third partial area 1446 as a partial area where a check is to be entered.

Conventionally, a problem list for nurses and a problem list for doctors have been prepared for the entry of problems in problem lists like this. Recently, however, there are increasing cases where medical care is addressed by a team of doctors and nurses, for it makes little difference to the patients. Accordingly, there are increasing cases where the same problem list is used by both the doctors and nurses, as seen with a problem list 1376 illustrated in FIG. 42. In such a case, problems to be entered in the problem list includes problems to be dealt with by both doctors and nurses and problems to be dealt with by nurses only. In this, the problems to be dealt with by both doctors and nurses are called common problems, whereas the problems to be dealt with by nurses only are called nursing problems. And concerning the common problems, the doctors and nurses must carry out their medical treatment activities as a team. Hence, in the entry of problems in the problem list, it is necessary to make a clear distinction between the common problems and the nursing problems.

Thus, in an electronic patient's chart system 1300 according to the nineteenth embodiment, a predefined processing is executed on medical information data representing the common problems by entering a specification mark, such as a check, in a check space 1380 thereby specifying the problems corresponding to the check as common problems. As a result, the doctors and nurses can easily distinguish between the nursing problems and the common problems and accordingly respond to them easily by using the medical information data effectively.

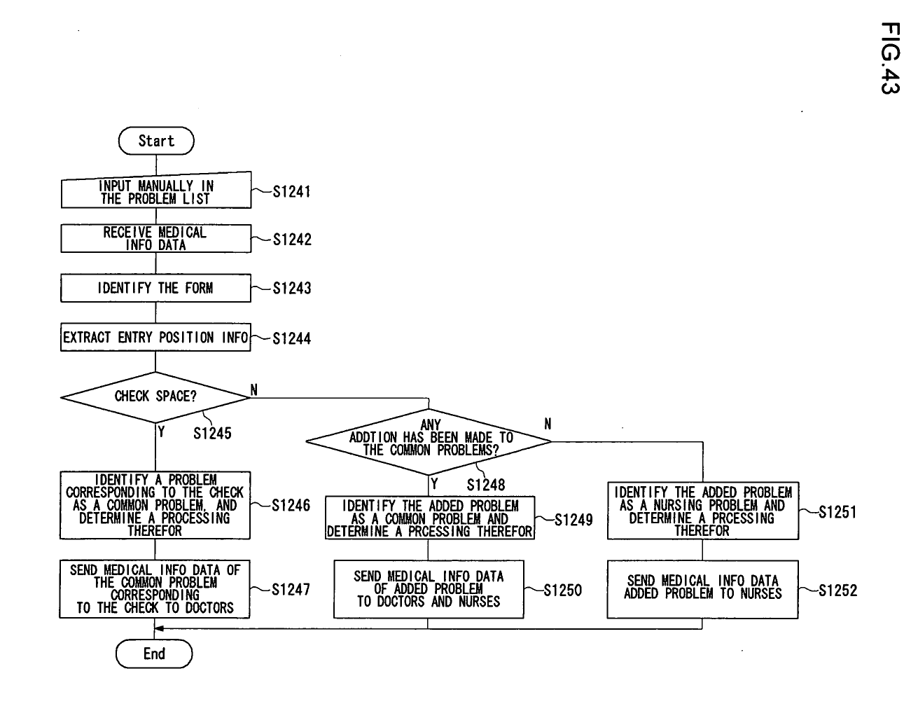

FIG. 43 is a flowchart showing the processing of an electronic patient's chart system 1300 according to the nineteenth embodiment. A nurse or a physician manually inputs medical information data, representing problems or a check, in a problem list 1376 using an electronic pen 1012 (S1241). As the medical information data are manually inputted onto the problem list form 1376 with an electronic pen 1012, a workstation 1200 in the division concerned receives the medical information data acquired by the electronic pen 1012. A server 1100 receives the medical information data acquired by the electronic pen 1012 from the workstation 1200 via a network 1050 (S1242).

As the server 1100 acquires the medical information data, a form identification unit 1132 extracts a form ID from the acquired medical information data and identifies a form 1010 entered with medical information represented by the medical information data by using the extracted form ID and a form table 1112 stored in a storage unit 1110 (S1243). As a result, a specific patient's problem list form 1376 in which the medical information data have been manually inputted is identified, and form information corresponding to this problem list form 1376 out of form information stored in the storage unit 1110 is identified. Also, the form identification unit 1132 identifies a form corresponding to this form information by identifying the form information. The received medical information data are associated with the identified form information and stored in the storage unit 1110.

As the form information is identified, an entry position extraction unit 1138 extracts the entry position information from the acquired medical information data (S1244). A partial area extraction unit 1140 extracts partial area information for the manual input of the acquired medical information data by using the extracted entry position information. By deciding whether the extracted partial area information represents the twenty-third partial area 1446 or not, a processing determination unit 1150 decides whether a check is placed in the check space 1380 or not (S1245).

When it is decided that a check is placed in the check space 1380 (Y of S1245), the processing determination unit 1150 refers to a data processing table 1118, identifies the problems corresponding to the check as common problems, and decides the subsequent processing (S1246). In this case, the processing determination unit 1150 identifies the position of check entry by using the input position of the medical information data. The processing determination unit 1150 identifies the medical information data representing the date entered in a position closest to the position where the check is entered, out of the dates entered in the date space 1382. The processing determination unit 1150 further identifies the medical information data representing the nursing record corresponding to this date. In this manner, the processing determination unit 1150 identifies the medical information data of the nursing record corresponding to the check. As the problems corresponding to the check are identified as common problems, a transmission processing unit 1162 transmits the medical information data of the common problems to doctors (S1247). As a result, it is possible to communicate common problems to physicians by using the medical information data effectively.

When it is decided that no check is placed in the check space 1380 (N of S1245), the processing determination unit 1150 decides that an additional entry has been made in one of the date space 1382, the problem space 1384 and the addition/correction space 1386 and further decides whether any addition has been made to the common problems or not (S1248). As mentioned above, the processing determination unit 1150 decides whether any addition has been made to the common problems or not by deciding whether any addition has been entered near the problems identified as the common problems. When it is decided that any additional entry has been made to the common problems (Y of S1248), the processing determination unit 1150 refers to the data processing table 1118, identifies the added problem as a common problem, and decides the subsequent processing (S1249). As the added problem is identified as a common problem, the transmission processing unit 1162 transmits the medical information data of the added problem to doctors and nurses (S1250).

When it is decided that no additional entry has been made to the common problems (N of S1248), the processing determination unit 1150 refers to the data processing table 1118, identifies the added problem as a nursing problem first, and decides the subsequent processing (S1251). As the added problem is identified as a nursing problem, the transmission processing unit 1162 transmits the medical information data of the added problem to nurses (S1252). As a result, a problem entered in the problem list 1376 can be first treated as a nursing problem and can be transmitted to nurses. When the problem entered in the problem list 1376 is a common problem, the medical information data of the common problem can be transmitted to doctors by later entering a check in the check space 1380. As a result, the doctors and nurses can easily distinguish between the nursing problems and the common problems and accordingly respond to them easily by using the medical information data effectively.

### Twentieth Embodiment

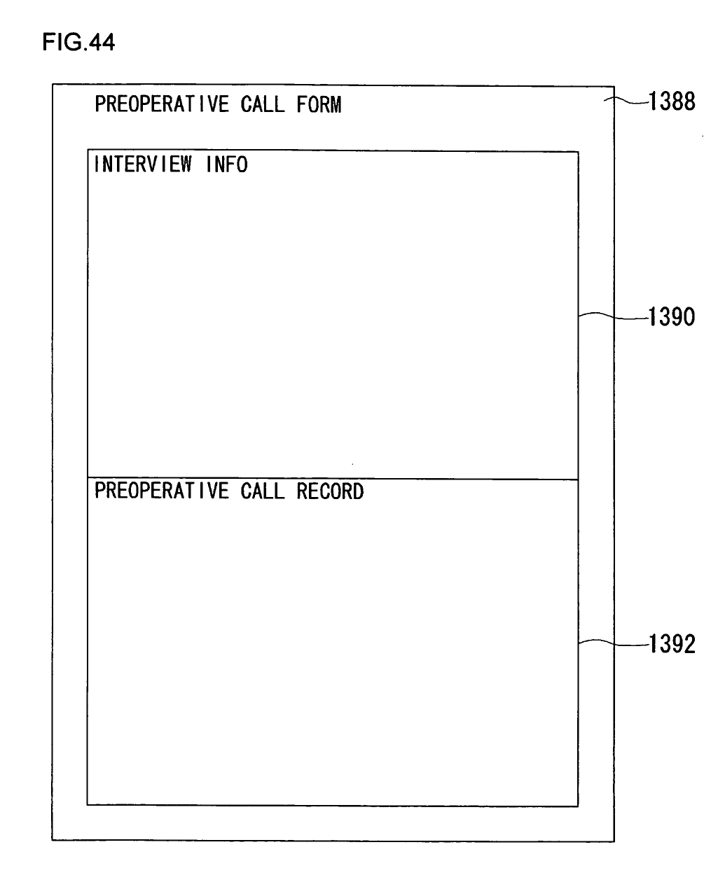

FIG. 44 illustrates a preoperative call form 1388 used in an electronic patient's chart system 1300 according to the twentieth embodiment. The preoperative call form 1388 is prepared in such a manner that the form of a preoperative call form 1388 is printed on a form 1010 already printed with a dot pattern which is readable by an electronic pen 1012. The preoperative call form 1388 includes an interview information space 1390 and a preoperative call record space 1392.

When, for instance, a patient is admitted to a hospital, a nurse conducts a history-taking interview, in which the patient is directly asked about the present health condition and the like. This interview can clarify the patient's allergy or other history as well as present conditions. On the other hand, preceding a surgical operation, a nurse makes a call to the patient and checks the conditions of the patient. Normally, a nurse who conducts a history-taking interview and a nurse who makes a preoperative call to the patient are different nurses from different hospital wards. Consequently, when a patient who is currently or formerly an inpatient is to undergo an operation, there are possibilities that the patient's condition checks are repeated unnecessarily. In the electronic patient's chart system 1300 according to the twentieth embodiment, therefore, the interview information as information obtained in a previous history-taking interview is printed in advance on the preoperative call form 1388 to which entries are to be made on a preoperative call to the patient.

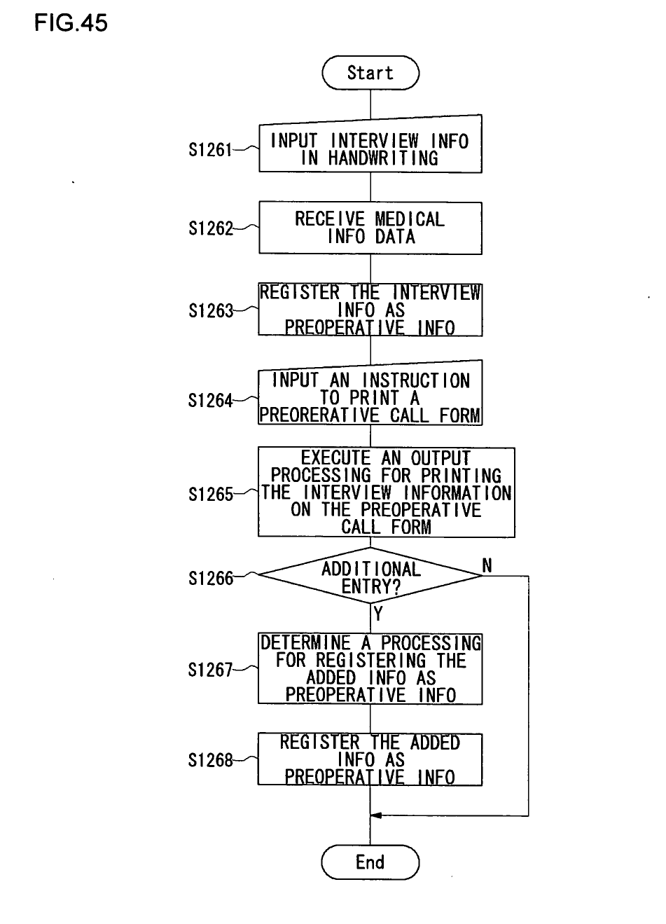

FIG. 45 is a flowchart showing the processing of an electronic patient's chart system 1300 according to the twentieth embodiment. A nurse in a ward manually inputs interview information onto a prescribed interview information recording form using an electronic pen 1012 (S1261). This interview information recording form is prepared in such a manner that the form of a interview information recording sheet is printed on a form 1010 already printed with a dot pattern which is readable by an electronic pen 1012. As the medical information data are manually inputted onto the interview information recording form with an electronic pen 1012, a workstation 1200 in the division receives the medical information data acquired by the electronic pen 1012. A server 1100 receives the medical information data acquired by the electronic pen 1012 from the workstation 1200 via a network 1050 (S1262). Upon receipt of the medical information data, a registration processing unit 1164 registers the interview information as preoperative information (S1263).

When the input of an instruction to print a preoperative call form is made by a user after the interview information is registered as preoperative information (S1264), an output processing unit 1166 executes an output processing for printing the interview information on the preoperative call form (S1265). The output processing unit 1166 prints the interview information in the interview information space 1390 as with the preoperative call form 1388 illustrated in FIG. 44.

After the output processing for printing the interview information on the preoperative call form is executed, the processing determination unit 1150 decides whether any additional entry has been made to the preoperative call form 1388 or not (S1266). When no additional entry has been made to the preoperative call form 1388 (N of S1266), the processing determination unit 1150 skips the processing of registering the medical information data as preoperative information. When any additional entry has been made to the preoperative call form 1388 (Y of S1266), the processing determination unit 1150 refers to the data processing table 1118 and decides on the processing of registering the added information as preoperative information (S1267). As the processing of registering the added information as preoperative information is decided on, a registration processing unit 1164 saves the added information as preoperative information (S1268). The registration processing unit 1164 registers the added information as preoperative information by adding the preoperative information identification data representing the preoperative information to the medical information data representing the preoperative information before storing them in the storage unit 1110. This arrangement can save the duplicated labor of nurses. Also, by registering the information entered in a preoperative call form 1388 as preoperative information, it also becomes possible to display the preoperative information in a display mode different from that for the interview information, thus making it easier to distinguish between the interview information and preoperative information.

### Twenty-First Embodiment

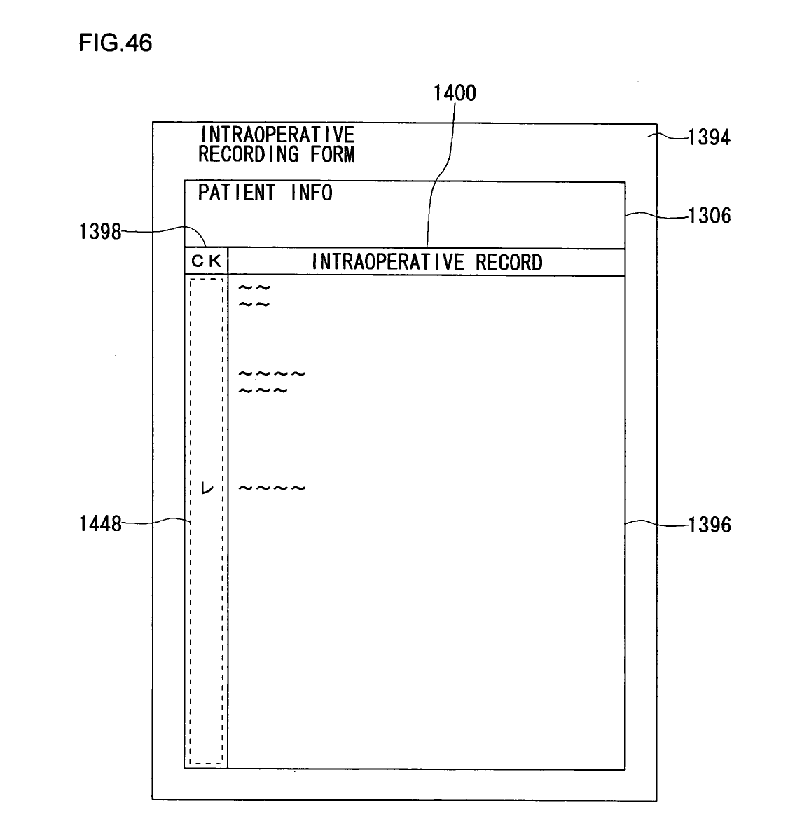

FIG. 46 illustrates an intraoperative record form 1394 used in an electronic patient's chart system 1300 according to a twenty-first embodiment. The intraoperative record form 1394 is prepared in such a manner that the form of an intraoperative record form 1394 is printed on a form 1010 already printed with a dot pattern which is readable by an electronic pen 1012. An intraoperative call record space 1394 includes a patient information space 1306 and an intraopertive record information space 1396. The intraopertive record information space 1396 is composed of a check space 1398 and an intraoperative record space 1400, which are arranged horizontally. Provided in the check space 1398 is a twenty-fourth partial area 1448 as a partial area for check entry.

An operating room chief nurse enters intraoperative record as shown in the intraoperative record form 1394. A surgical operation is normally carried out according to plan, and, in such a case, the nurse enters records concerning the planned operation in an intraoperative record form 1394. If the records are those of an ordinary planned operation, ward nurses can easily comprehend postoperative cares from the information. However, when the patient takes a sudden turn for the worse, for instance, temporary responses, such as dosage of temporary drugs, may take place in the course of the operation. Even when such temporary responses are conducted, the operating room chief nurse has to make necessary entries in the intraoperative record form 1394, but, in such a case, ward nurses may have difficulty in comprehending necessary postoperative cares from the information. Accordingly, when such emergency responses are conducted, the operating room chief nurse must carefully communicate the necessary postoperative cares to the ward nurses. Hence, the electronic patient's chart system according to the twenty-first embodiment registers as temporary information the medical information data representing the records checked in the check space 1398 out of the records entered in the intraoperative record form 1394.

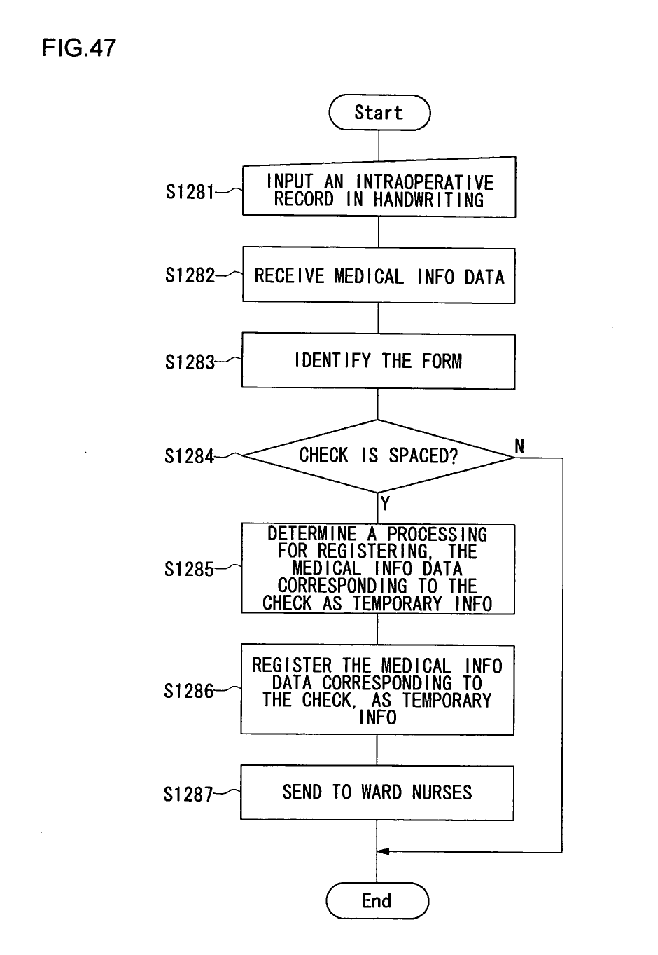

FIG. 47 is a flowchart showing the processing of an electronic patient's chart system 1300 according to the twenty-first embodiment.

An operating room nurse manually inputs medical information data during an operation by entering intraoperative records onto an intraoperative record form 1394 using an electronic pen 1012 (S1281). As the medical information data are manually inputted, a workstation 1200 in the division receives the medical information data acquired by the electronic pen 1012. A server 1100 receives the medical information data acquired by the electronic pen 1012 from the workstation 1200 via a network 1050 (S1282). As the medical information data are received, a form identification unit 1132 extracts a form ID from the acquired medical information data and identifies a form 1010 entered with medical information represented by the medical information data by using the extracted form ID and a form table 1112 stored in a storage unit 1110 (S1283). As a result, a specific patient's intraoperative record form 1394 in which the medical information data have been manually inputted is identified, and form information corresponding to this intraoperative record form 1394 out of form information stored in the storage unit 1110 is identified. Also, the form identification unit 1132 identifies a form corresponding to this form information by identifying the form information. The received medical information data are associated with the identified form information and stored in the storage unit 1110.

As the form information is identified, a processing determination unit 1150 decides whether a check is placed in the check space 1398 or not (S1284). As the medical information data are received, a partial area extraction unit 1140 extracts partial area information of the acquired medical information data. By using the extracted partial area information, the processing determination unit 1150 decides whether the acquired medical information data have been manually inputted in a twenty-fourth partial area 1448 or not. By this, the processing determination unit 1150 decides whether a check is placed in the check space 1398 or not. When it is decided that no check is placed in the check space 1398 (N of S1284), the processing determination unit 1150 decides that there have been no temporary responses during the operation and thereby terminates the processing in this flowchart.

When it is decided that a check is placed in the check space 1398 (Y of S1284), the processing determination unit 1150 refers to a data processing table 1118 and decides on the processing for registering as temporary, or emergency, information the medical information data of the intraoperative records corresponding to the check (S1285). The processing determination unit 1150 identifies the position of check entry by using the input position of the medical information data. The processing determination unit 1150 identifies the medical information data representing the intraoperative records entered in a position closest to the position where the check is entered, out of the series of intraoperative records entered in the intraoperative record space 1400. In this manner, the processing determination unit 1150 identifies the medical information data of the intraoperative records corresponding to the check.

As the processing for registering as temporary, or emergency, information the medical information data of the intraoperative records corresponding to the check is decided on, a registration processing unit 1164 registers the medical information data of the checked intraoperative records as temporary information (S1286). The registration processing unit 1164 registers the medical information data of the checked intraoperative records as temporary information by adding the temporary information identification data representing the temporary information to the medical information data before storing them in the storage unit 1110. As the medical information data of the checked intraoperative records is saved as temporary information, a transmission processing unit 1162 transmits the medical information data representing the intraoperative records entered in the intraoperative record space 1400 to ward nurses (S1287). At this time, an output processing unit 1218 of the workstation 1200 displays the temporary information in a display mode different from that for information other than temporary information by using the temporary information identification data added to the medical information data. As a result, the ward nurses can easily recognize the temporary, or emergency, information in distinction from the intraoperative records entered in the intraoperative record form 1394 and comprehend the temporary, or emergency, information without fail.

The present invention is not limited to each of the above-described embodiments and the combination of each constituent element in each embodiment may be combined as appropriate and the combination thereof may serve effective as an embodiment of the present invention. It is also possible to add modification with a variety of changes in designing and so forth to each embodiment, based on the knowledge of the skill in the art. The embodiments added with such modifications are also within the scope of the present invention. Such modification will be described hereinbelow.

The electronic patient's chart system 1300 may acquire the medical information data representing the manually inputted medical information by use of a scanner. In such a case, the form will function as an input medium; the surface of paper will function as input area; and the scanner will function as a means for acquiring medical information data. Also, the electronic patient's chart system 1300 may acquire the medical information data representing the manually inputted medical information by use of a graphic tablet, a digitizer, or a tablet PC capable of inputting to the screen. In such a case, each of these devices will function as an input medium and a means for acquiring medical information data, and the input surface and screen of these devices will function as input area. As a result, it is possible to easily acquire the manually inputted medical information as medical information data.

What is claimed is:

1. A medical information management system, comprising:
a medical information data acquisition unit which acquires medical information data inputted in handwriting;
a graphic area identification unit which identifies a graphic area defined by a graphic represented by graphic data contained in the medical information, based on the acquired medical information data;
a comment area identification unit which identifies a comment area located in a predetermined position relative to the identified graphic area;
a comment data identification unit which identifies comment data inputted in handwriting to the comment area, among the acquired medical information data; and
a medical information storage unit which stores the acquired medical information data and the identified comment data in a manner that associates the acquired medical information data with the identified comment data.

2. A medical information management system according to claim 1, further comprising a lead line data identification unit which identifies lead line data indicative of a lead line connecting the graphic area with the comment area,
wherein, based on the identified lead line data, said comment data identification unit identifies the extracted comment data, inputted in handwriting to the comment area, among the medical information data.

3. A medical information management system, comprising:
a medical information data acquisition unit which acquires medical information data inputted in handwriting;
a graphic area storage unit which stores graphic area data indicative of a graphic area defined by a graphic indicated beforehand in an input area where the medical information data are inputted in handwriting;
a comment area identification unit which identifies a comment area located in a predetermined position relative to the graphic area indicated by the graphic area data stored in said graphic area storage unit;
a comment data identification unit which identifies comment data inputted in handwriting to the comment area, among the medical information data; and
a medical information storage unit which stores the acquired medical information data and the identified comment data in a manner that associates the acquired medical information data with the identified comment data.

4. A medical information management system according to claim 3, further comprising a graphic area identifying data storage unit which stores graphic area identifying data that identifies the graphic area indicated by the graphic area data,
wherein said medical information storage unit stores the acquired medical information data and the graphic area identifying data, based on a position at which the comment data have been inputted in handwriting, in a manner that associates the acquired medical information data with the graphic area identifying data.

5. A medical information management system according to claim 3, further comprising:
a partial area storage unit which stores partial area data indicative of a partial area contained in the graphic area; and
a partial area identifying data storage unit which stores partial area identifying data that identifies a partial area indicated by the partial area data,
wherein said medical information storage unit stores the acquired medical information data and the partial area identifying data, based on a position at which the comment data have been inputted in handwriting, in a manner that associates the acquired medical information data with the partial area identifying data.

6. A medical information management system according to claim 4, further comprising:
a partial area storage unit which stores partial area data indicative of a partial area contained in the graphic area; and
a partial area identifying data storage unit which stores partial area identifying data that identifies a partial area indicated by the partial area data,
wherein said medical information storage unit stores the acquired medical information data and the partial area identifying data, based on a position at which the comment data have been inputted in handwriting, in a manner that associates the acquired medical information data with the partial area identifying data.

7. A method for managing medical information, the method comprising:
acquiring medical information data inputted in handwriting;
identifying a graphic area defined by a graphic represented by graphic data contained in the medical information, based on the acquired medical information data;
identifying a comment area located in a predetermined position relative to the identified graphic area;
identifying comment data inputted in handwriting to the comment area, among the acquired medical information data; and
storing the acquired medical information data and the identified comment data in a manner that associates the acquired medical information data with the identified comment data.

8. A method for managing medical information, the method comprising:
acquiring medical information data inputted in handwriting;
storing graphic area data indicative of a graphic area defined by a graphic indicated beforehand in an input area where the medical information data are inputted in handwriting;
identifying a comment area located in a predetermined position relative to the graphic area indicated by the graphic area data stored in said storing;
identifying comment data inputted in handwriting to the comment area, among the medical information data; and
storing the acquired medical information data and the identified comment data in a manner that associates the acquired medical information data with the identified comment data.

9. A medical information management system, comprising:
a medical information data acquisition unit which acquires medical information data inputted in handwriting;
an attribute extraction unit which extracts, from the acquired medical information data, attribute information that identifies a state in which the medical information data are inputted in handwriting;
a processing determination unit which determines a processing to be performed on the medical information data, using the extracted attribute information; and
a processing execution unit which performs the determined processing.

10. A medical information management system according to claim 9, further comprising a processing storage unit which stores data processing information that associates the attribute information with the processing to be performed on the medical information,
wherein said processing determination unit refers to the data processing information stored in said first processing storage unit, and determines performing a processing associated with the extracted attribute information, on the medical information data.

11. A medical information management system according to claim 9,
wherein the processing storage unit stores data processing information that associates a plurality of attribute information with the processing to be performed on the medical information,
wherein said attribute extraction unit extracts a plurality of attribute information from the acquired medical information data, and
wherein said processing determination unit refers to the data processing information stored in said storage unit, and determines performing a processing associated with the plurality of extracted attribute information, on the medical information data.

12. A medical information management system according to claim 11, wherein said processing storage unit stores entry-maker classification information indicative of a category of an entry-maker, partial area information indicative of a partial area in which the medical information data are inputted in handwriting and a processing to be performed on the medical information data in a manner that the entry-maker classification information and the partial area information are associated with the processing to be performed on the medical information data,
wherein said attribute extraction unit includes:
an entry-maker classification extraction unit which extracts the entry-maker classification information from the acquired medical information data;
an entry-maker position extraction unit which extracts entry position information indicative of a position, at which the acquired medical information data are inputted in handwriting, from the acquired medical information data; and
a partial area extraction unit which extracts the partial area information, indicative of a partial area in which the medical information data are inputted in handwriting, using the extracted entry position information, and
wherein said processing determination unit has a processing, associated with the extracted entry-maker classification information and the extracted partial area information, executed on the medical information data by referring to the data processing information stored.

13. A medical information management system according to claim 12, further comprising:
a data processing unit which processes the medical information data; and
a transmitter which transmits the medical information data to said data processing unit,
wherein said processing storage unit stores the entry-maker classification information, the partial area information and a transmission processing in which the medical information data are transmitted to said data processing unit by use of said transmitter, in a manner that the entry-maker classification information and the partial area information are associated with the transmission processing, and
wherein said processing determination unit has a processing, associated with the extracted entry-maker classification information and the extracted partial area information, executed on the medical information data by referring to the data processing information stored.

14. A medical information management system according to claim 9, further comprising:
a data processing unit which processes the medical information data; and
a transmitter which transmits the medical information data to said data processing unit,
wherein when said processing determination unit determines that the medical information data be transmitted to said data processing unit, said processing execution unit transmits the medical information data to said data processing unit by use of said transmitter.

15. A medical information management system according to claim 10, further comprising:
a data processing unit which processes the medical information data; and
a transmitter which transmits the medical information data to said data processing unit,
wherein when said processing determination unit determines that the medical information data be transmitted to said data processing unit, said processing execution unit transmits the medical information data to said data processing unit by use of said transmitter.

16. A medical information management system according to claim 11, further comprising:
a data processing unit which processes the medical information data; and
a transmitter which transmits the medical information data to said data processing unit,
wherein when said processing determination unit determines that the medical information data be transmitted to said data processing unit, said processing execution unit transmits the medical information data to said data processing unit by use of said transmitter.

17. A medical information management system according to claim 12, further comprising:
a data processing unit which processes the medical information data; and
a transmitter which transmits the medical information data to said data processing unit,
wherein when said processing determination unit determines that the medical information data be transmitted to said data processing unit, said processing execution unit transmits the medical information data to said data processing unit by use of said transmitter.

18. 14. A medical information management system according to claim 13, further comprising:
a data processing unit which processes the medical information data; and
a transmitter which transmits the medical information data to said data processing unit,
wherein when said processing determination unit determines that the medical information data be transmitted to said data processing unit, said processing execution unit transmits the medical information data to said data processing unit by use of said transmitter.

19. A medical information management system according to claim 9, further comprising:
a data processing unit which processes the medical information data; and
a transmitter which transmits the medical information data to said data processing unit,
wherein when said processing determination unit determines that the medical information data be transmitted to said data processing unit, said processing execution unit transmits the medical information data to said data processing unit by use of said transmitter, in accordance with a request from said data processing unit.

20. A medical information management system according to claim 10, further comprising:
a data processing unit which processes the medical information data; and
a transmitter which transmits the medical information data to said data processing unit,
wherein when said processing determination unit determines that the medical information data be transmitted to said data processing unit, said processing execution unit transmits the medical information data to said data processing unit by use of said transmitter, in accordance with a request from said data processing unit.

21. A medical information management system according to claim 11, further comprising:
a data processing unit which processes the medical information data; and
a transmitter which transmits the medical information data to said data processing unit,
wherein when said processing determination unit determines that the medical information data be transmitted to said data processing unit, said processing execution unit transmits the medical information data to said data processing unit by use of said transmitter, in accordance with a request from said data processing unit.

22. A medical information management system according to claim 12, further comprising:
a data processing unit which processes the medical information data; and
a transmitter which transmits the medical information data to said data processing unit,
wherein when said processing determination unit determines that the medical information data be transmitted to said data processing unit, said processing execution unit transmits the medical information data to said data processing unit by use of said transmitter, in accordance with a request from said data processing unit.

23. A medical information management system according to claim 13, further comprising:
a data processing unit which processes the medical information data; and
a transmitter which transmits the medical information data to said data processing unit,
wherein when said processing determination unit determines that the medical information data be transmitted to said data processing unit, said processing execution unit transmits the medical information data to said data processing unit by use of said transmitter, in accordance with a request from said data processing unit.

24. A medical information management system according to claim 14, further comprising:
a data processing unit which processes the medical information data; and
a transmitter which transmits the medical information data to said data processing unit,
wherein when said processing determination unit determines that the medical information data be transmitted to said data processing unit, said processing execution unit transmits the medical information data to said data processing unit by use of said transmitter, in accordance with a request from said data processing unit.

25. A method for processing medical information, the method comprising:
acquiring medical information data inputted in handwriting;
extracting, from the acquired medical information data, attribute information that identifies a state in which the medical information data are inputted in handwriting;
determining a processing to be performed on the medical information data, using the extracted attribute information; and
executing the determined processing.
